(19) United States
(12) Patent Application Publication
(10) Pub. No.: US 2024/0016784 A1
(43) Pub. Date: Jan. 18, 2024

(54) Y BOX BINDING PROTEIN 1 INHIBITORS

(71) Applicants: Oregon Health & Science University, Portland, OR (US); The Board of Trustees of the Leland Stanford Junior University, Stanford, CA (US)

(72) Inventors: Sanjay V. Malhotra, Portland, OR (US); Dhanir Tailor, Portland, OR (US); Arpit Dheeraj, Portland, OR (US)

(73) Assignees: Oregon Health & Science University, Portland, OR (US); The Board of Trustees of the Leland Stanford Junior University, Stanford, CA (US)

(21) Appl. No.: 18/255,827

(22) PCT Filed: Dec. 3, 2021
(86) PCT No.: PCT/US2021/061906

§ 371 Date: Jun. 2, 2023

Related U.S. Application Data
(60) Provisional application No. 63/256,609, filed on Oct. 17, 2021.
(60) Provisional application No. 63/121,674, filed on Dec. 4, 2020.

(51) Int. Cl. A61K 31/4355; A61P 35/00

(52) U.S. Cl. CPC A61K 31/4355; A61P 35/00

(57) ABSTRACT

Provided herein are novel azopodophyllotoxin analog compounds, pharmaceutical compositions comprising them, and their use as inhibitors of Y box protein 1 (YB1 or YBX1) in treatments for conditions including gynecological, breast, bladder, kidney, head and neck, neuronal, and prostate cancers, lymphomas, and leukemias. Methods of their use in sensitizing resistant cancers to treatment with anticancer agents and radiation are also provided.

### CROSS-REFERENCE TO RELATED APPLICATIONS

This is the 371 National Phase of International Application No. PCT/US2021/061906, filed Dec. 3, 2021, which claims priority to and the benefit of the earlier filing of both U.S. Provisional Application No. 63/121,674, filed on Dec. 4, 2020, and U.S. Provisional Application No. 63/256,609, filed Oct. 17, 2021, both of which are incorporated by reference herein in their entirety.

### FIELD OF THE INVENTION

The present invention concerns novel compounds, pharmaceutical compositions, and their use as inhibitors of Y box protein 1 (YB1 or YBX1) to treat conditions including gynecological, breast, bladder, kidney, head and neck, neuronal, and prostate cancers, lymphomas, and leukemias.

### BACKGROUND OF THE INVENTION

Y box binding protein 1 (YB-1) is a multifunctional protein associated with tumor progression and the emergence of treatment resistance (TR). Here, we report an azopodophyllotoxin small molecule, SU056, that potently inhibits tumor growth and progression via YB-1 inhibition. This first-in-class YB-1 inhibitor inhibits cell proliferation, resistance to apoptosis in ovarian cancer (OC) cells and arrests in the G1 phase. Inhibitor treatment leads to enrichment of proteins associated with apoptosis and RNA degradation pathways while downregulating spliceosome pathway. In vivo, SU056 independently restrains OC progression and exerts a synergistic effect with paclitaxel to further reduce disease progression with no observable liver toxicity. Moreover, in vitro mechanistic studies showed delayed disease progression via inhibition of drug efflux and multi-drug resistance 1 (MDR1), and significantly lower neurotoxicity as compared to Etoposide. These data suggest that YB-1 inhibition may be an effective strategy to reduce OC progression, antagonize TR, and decrease patient mortality.

Y box binding protein 1 (YB-1, YBX1) is a multifunctional cold shock protein that binds to DNA and RNA. It regulates DNA and RNA associated cellular events including mRNA transcription, splicing, packaging, stability, and translation (Lyabin et al., 2014). mRNA stabilization is an important event for sustained expression of any gene and YB-1 robustly stabilizes the mRNA via blocking the 5′ end from mRNA degradation (Evdokimova et al., 2001). It was first described by Didier et al. as a negative regulator of the MHC class II molecule (Didier et al., 1988). The oncogenic role of YB-1 is well-characterized in many cancers and its amplified levels have been found in a large number of cancer cases (Goodarzi et al., 2015). It increases the stability of short-lived mRNAs for multiple oncogenic proteins including c-myc (Laird-Offringa et al., 1990), c-fos (Blattner et al., 2000), cyclin B1 (Maity et al., 1995), HIF1α (Goodarzi et al., 2015), Snail (Evdokimova et al., 2009), and MDR1 (Bargou et al., 1997), which are associated with disease progression and treatment resistance. Genetic knockdown studies have demonstrated that inhibition of YB-1 significantly arrests proliferation and induces apoptosis in many cancer models, demonstrating its essential role in disease progression (Evdokimova et al., 2009, El-Naggar et al., 2015). YB-1 is associated with the development of treatment resistance (TR) via its role in activating proliferation, promoting cancer cell stemness, responding to growth factors, cytokines, cellular stress responses, and promoting drug efflux via the membrane P-glycoprotein ATP-dependent efflux pump ABCB1 (MDR1) (Bargou et al., 1997, Saupe et al., 2015, Mo et al., 2016). It is also associated with alternative splicing of CD44 exon via binding to the A/C-rich region (Stickeler et al., 2001). The YB-1 gene is highly conserved and only ˜-1% of cancer patients show the mutation, although it is nonetheless overexpressed in a wide range of cancers via alternative gene regulatory networks.

Ovarian cancer (OC) accounts for only 3% of all cancer cases in women, but nonetheless causes disproportionate mortality (Dietl, 2014, Jayson et al., 2014, Agarwal and Kaye, 2003). Surgical resection followed by chemotherapy is the main treatment strategy for OC patients. Platinum- and taxol-based drugs and their combination are the first-line treatment for the majority of OC patients (Seifter, 1997). The majority of women are diagnosed with OC at Stage 111+ and frequently develop TR and disease relapse. BRCA1/2 mutations, amplification of MYC, and upregulation MDR1 (ABCB1/P-gp) are the most common known causes of TR in OC (Zeng et al., 2018, Christie and Bowtell, 2017, Sun et al., 2015). Patient-based studies have shown that MYC amplification is associated with disease progression and TR in many high-grade epithelial OC lesions (Jung et al., 2017, Jung et al., 2018). Nuclear localization of YB-1 plays an important role in the regulation of MYC, MDR1, and CD44 (Kang et al., 2013, Sobocan et al., 2020). Analysis of high-grade ovarian serous carcinoma samples suggests that patients with higher YB-1 expression (median survival 48.5 months) had shorter term survival compared to lower YB-1 expression (median survival 65 months) (Kang et al., 2013). Primary surgical and chemotherapy treatments for OC may be followed by maintenance therapy, which includes prolonged drug usage such as paclitaxel and PARP inhibitors (olaparib, pazopanib, and niraparib), but unfortunately still with limited outcomes (Franzese et al., 2019). Literature from the last three decades strongly suggests that YB-1 could be a potential target to treat ovarian and other cancers including those in which treatment resistance has developed. Even after an extensive investigation, there were no significant efforts had been made to develop small molecule inhibitors that can directly inhibit the YB-1. Y-box-binding protein 1 (YB-1), encoded by the YBX1 gene, has been noted as modulating or regulating cellular signaling pathways and may be seen as a molecular marker for cancer progression and as a target for cancer therapies.

Lasham et al. describe in their review article YB-1: oncoprotein, prognostic marker and therapeutic target?, Biochem. J. (2013) 449, 11-13, how “YB-1 regulates multiple proliferation pathways, overrides cell-cycle check points, promotes replicative immortality and genomic instability, may regulate angiogenesis, has a role in invasion and metastasis, and promotes inflammation.” They further describe cell lines in which YB-1 reduction induced apoptosis or inhibited cell proliferation, including melanoma, fibrosarcoma, liver cancer, lung cancer, bladder cancer, multiple myeloma, pediatric glioblastoma, breast cancer (ER-negative), breast cancer (ER-positive), prostate cancer, and colon cancer cell lines.

Sobočan et al. (Cancers, 2020, 12, 205) describe dual targeting of Y-box-protein 1 (YB-1) and mTOR as improving the inhibition of carcinogenic activity in gynecological cancers, including ovarian, endometrial, fallopian tube, and cervical cancers.

The article Oncogenic Y-box binding protein-1 as an effective therapeutic target in drug-resistant cancer, Kuwano et al., Cancer Science, 2019, 110:1536-1543, describes the function of YBX2 in promoting transcriptional activation of the ABCB1 transporter gene, which has been associated as a transcriptional mechanism of how tumor multidrug resistance is acquired during chemotherapeutic treatments in human malignancies, including breast, lung, ovarian, prostate, colorectal, and gastric cancers.

The relationship between increased expression of YBX1 and melanoma is discussed in the article The increased expression of Y box-binding protein 1 in melanoma stimulates proliferation and tumor invasion, antagonizes apoptosis and enhances chemoresistance, Schittek et al., Int. J. Cancer: 120, 2110-2118 (2007). YB1 overexpression has also been associated with radio-resistance in colorectal cancer cells, as discussed by Kim et al., Mol. Cancer Ther., 30 Oct. 2019, 19(2), 479-89.

WO 2019/178091 A1 (Malholtra et al., The Board of Trustees of the Leland Stanford Junior University) teaches novel N-hydroxyethyl didehydroazapodophyllotoxins as GBP1 inhibitors and methods for their use in overcoming treatment resistance in cancers. Included in the disclosure are the specific compounds 9-(3-fluorophenyl)-5-(2-hydroxyethyl)-6,9-dihydro-[1,3]dioxolo[4,5-g]furo[3,4-b]quinolin-8(5H)-one (SU056); 5-(2-hydroxyethyl)-9-(3-(trifluoromethyl)phenyl)-6,9-dihydro-[1,3]dioxolo[4,5-g]furo[3,4-b]quinolin-8(5H)-one; 3-(5-(2-hydroxyethyl)-8-oxo-5,6,8,9-tetrahydro-[1,3]dioxolo[4,5-g]furo[3,4-b]quinolin-9-yl)benzonitrile; and 5-(2-hydroxyethyl)-9-(pyridin-4-yl)-6,9-dihydro-[1,3]dioxolo[4,5-g]furo[3,4-b]quinolin-8(5H)-one.

There remains a need for small molecule inhibitors of YB-1 for pharmaceutical use.

### SUMMARY OF THE INVENTION

Bioisosterism is considered a crucial tool for rational drug design as medicinal chemists can rapidly manipulate a lead structure to optimize potency and selectivity, absorption, distribution, metabolism, and excretion (ADME) properties. Herein, we used bioisostere replacement to optimize the anti-OC effects of a lead compound using structure-guided approaches followed by target identification via Cellular Thermal Shift Assay (CETSA) (Savitski et al., 2014). Through in vitro and in vivo studies, we showed that the azopodophyllotoxin (AzP) small molecule SU056 potently inhibits YB-1 and reduces OC progression while sensitizing to chemotherapy-mediated cytotoxicity.

One embodiment provides a compound of Formula (I),

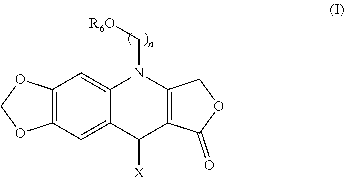

wherein:
X is selected from the group of:

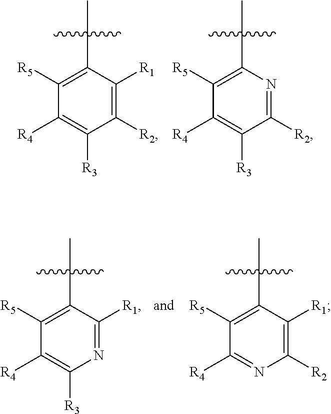

R₁, R₂, R₃, R₄, and R₅ are each independently selected in each instance from the group of H, F, C₁-C₄ fluoroalkyl, SF₅, Cl, Br, I, OH, C₁-C₄ alkyl, C₁-C₄ alkoxy, ON, NO₂, and OH; n is an integer selected independently in each instance from the group of 1, 2, 3, 4, 5, and 6; R₆ is selected from the group of H, C₁-C₆ alkyl, C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl; wherein the C₁-C₆ alkyl group is substituted with 0, 1, 2, 3, or 4 substituents selected from F, Cl, Br, I, OH, ON, NO₂, and OH; and the C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl groups are substituted with 0, 1, 2, 3, or 4 substituents selected from the group of C₁-C₃ alkyl, C₁-C₃ alkoxy, F, Cl, Br, I, OH, ON, NO₂, and OH; with the proviso that at least one of R₁, R₂, R₃, R₄, and R₅ is selected from the group of F, C₁-C₄ fluoroalkyl, and SF₅; and
with the proviso that, when X is

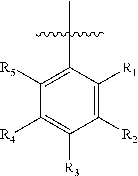

and R₂ is F or CF₃, then at least one of R₁, R₃, R₄, and R₅ is not H; or a pharmaceutically acceptable salt, pharmaceutically acceptable co-crystals, pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs thereof.

### BRIEF DESCRIPTION OF THE DRAWINGS

FIG. 1A depicts conversion of SU093 to SU056.

FIG. 1B charts IC₅₀ values of SU093 and SU056 on various ovarian cancer cells. FIG. 1C presents a photo of colony formation from respective wells.

FIG. 1D graphs the number of colonies formed after SU093 and SU056 treatment.

FIG. 1E provides a table representing % inhibition values of etoposide, SU093, and SU056 treatment in neuronal (SH-SY5Y, N27) and HEK293 cells.

FIG. 1F presents a table of cell cycle distribution of propidium iodide (PI)-stained OVCAR8, SKOV3, and ID8 cells.

FIG. 1G graphs effects of SU093 and SU056 on apoptotic cell death.

FIG. 1H graphs results of a cell migration assay.

FIG. 2A presents representative images of tumor regression compared to control in mice after 42 days of drug treatment.

FIG. 2B graphs tumor volume/mouse as a function of time.

FIG. 2C graphs tumor weight/mouse at the end of the study.

FIG. 2D provides a graph of liver toxicity parameters at the end of 42 days showing no significant difference between control, SU093, and SU056.

FIG. 2E presents an image of H&E staining from a lung metastasis assay.

FIG. 2F provides a graph of the number of lung metastatic nodules.

FIG. 3A provides a heat map representation of the thermal stability of 804 soluble proteins in ovarian cancer cells treated with vehicle-DMSO (left) and SU056 (right).

FIG. 3B graphs density distributions of protein Tm values calculated in SU056 treated cells and vehicle cells.

FIG. 3C presents a graph of density distributions of Tm shifts between SU056 and vehicle treatment.

FIG. 3D provides a scatter plot of Tm calculated in SU056 and vehicle treatment.

FIG. 3E presents melting curves for six proteins with and without SU056 treatment.

FIG. 3F provides a chart of change in melting temperature (Tm) of the top six proteins upon SU056 treatment.

FIG. 4A depicts a Western blot analysis was performed for the top three targets identified by CETSA.

FIG. 4B provides subset images for immunohistochemistry of tumor samples from ID8 tumor xenograft study.

FIG. 4C shows a graph of % inhibition in respective OC cells treated with SU056 and YB-1.

FIG. 4D presents a graph of YB-1 (IC50) of SU056 for OC cell.

FIG. 4E presents graphs depicting YB-1 inhibition time kinetics study for SU056 effect on OC cell lines.

FIG. 5A presents the structure of biotinylated SU056.

FIG. 5B depicts a pulldown assay using biotinylated SU056.

FIG. 5C provides a representative sensogram for SU093.

FIG. 5D provides a representative sensogram for S SU056.

FIG. 5E presents a Western blot analysis conforming YB-1 expression in transduced cells.

FIG. 5F presents an image demonstrating the cellular effect of SU056 is dependent on YB-1 expression.

FIG. 5G provides a graph of IC₅₀ values of SU056 on different transduced OVCAR8 cells expressing SC, YBX1 shRNA1, YBX1 shRNA2.

FIG. 6A depicts a cycloheximide chase assay to determine the effect of SU056 on YB-1 protein stability and a graph of fold change over time.

FIG. 6B depicts a SDS-PAGE and Western blot analysis performed for YB-1, cell cycle, and apoptosis-associated markers.

FIG. 6C and FIG. 6D represent observed enrichment in the Apoptosis and RNA degradation pathway in proteins that increase in abundance upon treatment with SU056.

FIG. 6E provides a graph of enrichment in the Spliceosome pathways observed in proteins that decrease in abundance upon treatment with SU056.

FIG. 7A provides a chart representing the sensitizing effects of SU056 on the viability of OVCAR8 and SKOV3 cells in combination with paclitaxel treatment.

FIG. 7B graphs an Alexa Fluor-488-tagged paclitaxel efflux assay showing SU056 cotreatment inhibits paclitaxel efflux.

FIG. 7C presents an SDS-PAGE gel for immunoblotting of YB-1 and MDR1. OVCAR8 cells treated with either vehicle (C), paclitaxel, SU056 and paclitaxel+SU056.

FIG. 7D provides microscopic images of spheroid at 10× magnification

FIG. 7E provides graphs of spheroid formation quantified after 7 days of incubation

FIG. 7F presents representative images of mice after 28 days of drug treatment showing tumor regression compared to control.

FIG. 7G graphs tumor volume/mouse as a function of time.

FIG. 7H tumor weight/mouse at the end of the study. I) Immunohistochemistry staining. Tumor sections were stained with Ki67, and slides were scored for KI67 staining. Data shown are mean±SD from 5 mice in each group. *P<0.05, **P<0.01, ***P<0.001 compared with respective control.

FIG. 8A depicts a western blot analysis for cell lysates from control and SU056 treated cells exposed at 37 and 53° C. temperature and analyzed for expression of YB-1, TMSB10 and PSMB2.

FIG. 8B depicts micrographs of cells imaged using confocal microscope for the mCherry-YB1 after 3 h treatment of SU056 (2.5 & 5 μM) at 10× magnification.

FIG. 8C presents a graph representing 2500 cells treated with SU056

FIG. 9 presents graphs representing the sensitizing effects of SU056 on the viability of OVCAR8 and SKOV3 cells in combination with paclitaxel treatment.

FIG. 10 presents a graph representing pharmacokinetics of SU056.

FIG. 11A provides graphs representing the growth inhibitory effect of SU056 evaluated using MTT assay.

FIG. 11B provides an image representing colony formation from cells treated with SU056 and incubated further to 7-10 days.

FIG. 11C depicts graphs representing the number of colonies formed after SU056 treatment.

FIG. 11D provides graphs representing the effect of SU056 on cell cycle distribution in TNBC cells.

FIG. 12A presents SDS-PAGE and western blot analysis for SU056 treatment inhibition of protein translation associated molecules among MDA-MB-231 b cells.

FIG. 12B presents SDS-PAGE and western blot analysis for SU056 treatment inhibition of protein translation associated molecules among MDA-MB-468 cells

FIG. 12C presents SDS-PAGE and western blot analysis for SU056 treatment inhibition of protein translation associated molecules among SUM 159 cells.

FIG. 13A presents a graph of tumor volume (MDA-MB-231) as a function of time.

FIG. 13B presents a graph of tumor weight (MDA-MB-231) at the end of the study.

FIG. 13C presents a graph of body weight (MDA-MB-231) as a function of time.

FIG. 13D presents a graph of tumor volume (MDA-MB-468) as a function of time.

FIG. 13E presents a graph of tumor weight (MDA-MB-468) at the end of the study.

FIG. 13F presents a graph of body weight (MDA-MB-468) as a function of time.

FIG. 13G provides representative images of tumor (MDA-MB-231) at the end of study.

FIG. 13H provides representative images of tumor (MDA-MB-468) at the end of study.

FIG. 13I presents a graph representing tumor volume (SUTI151-PDX) as a function of time.

FIG. 13J presents a graph representing tumor weight (SUTI151-PDX) at the end of the study.

FIG. 13K presents a graph representing Body weight (SUTI151-PDX) as a function of time.

FIG. 14A presents a graph of tumor volume (4T1) as a function of time in SU056 inhibition in a 4T1 tumor xenograft in BALB/c.

FIG. 14B presents a graph of tumor weight (4T1) at the end of the study.

FIG. 14C presents a graph of body weight (4T1) as a function of time.

FIG. 14D provides representative images of tumor (4T1) at the end of study.

FIG. 14E presents a graph of tumor volume (4T1) as a function of time.

FIG. 14F presents a graph of tumor weight (4T1) at the end of the study.

FIG. 14G presents a graph of body weight (4T1) as a function of time.

FIG. 15A present a graph depicting SU056 treatment is well tolerated in mice and rat as reflected by change in body weight in mice.

FIG. 15B present a graph depicting SU056 treatment is well tolerated in mice and rat as reflected by change in body weight in rat.

FIG. 15C presents a table of data indicating SU056 treatment of different concentration did not cause death among mice.

FIG. 15D presents a table of data indicating SU056 treatment of different concentration did not cause death among rats.

FIG. 15E presents a graph showing SU056 had a mean half-life of 40 minutes.

FIG. 16 presents normalized expression measurements of YBX1 in depicted cell lines.

FIGS. 17, 18, and 19 represent SDS-PAGE and western blots demonstrating SU056 treatment inhibited translation initiation factors in TNBC cells.

### DETAILED DESCRIPTION OF THE INVENTION

It is understood that, in the proviso above, that the rotational nature of phenyl rings that a compound in which R₂ is F or CF₃ and R₁, R₃, R₄, and R₅ are each H indicates the same substitution pattern as when R₄ is F or CF₃ and R₁, R₂, R₃, and R₅ are each H. Viewing the compound as having a F or CF₃ group at R₄ would indicate a proviso in which at least one of R₁, R₂, R₃, and R₅ is not H.

It is also understood that, within the scope of Formula (I) above, there are four additional and separate embodiments, each comprising a compound of Formula (Ia), Formula (Ib), Formula (Ic), or Formula (Id), respectively. In each embodiment, all variables (including n, R₁, R₂, R₃, R₄, R₅, and R₆), when present are as defined for Formula (I) above, with the compound of Formula (Ia) having the proviso that, when R₂ is F or CF₃, at least one of R₁, R₃, R₄, and R₅ is not H.

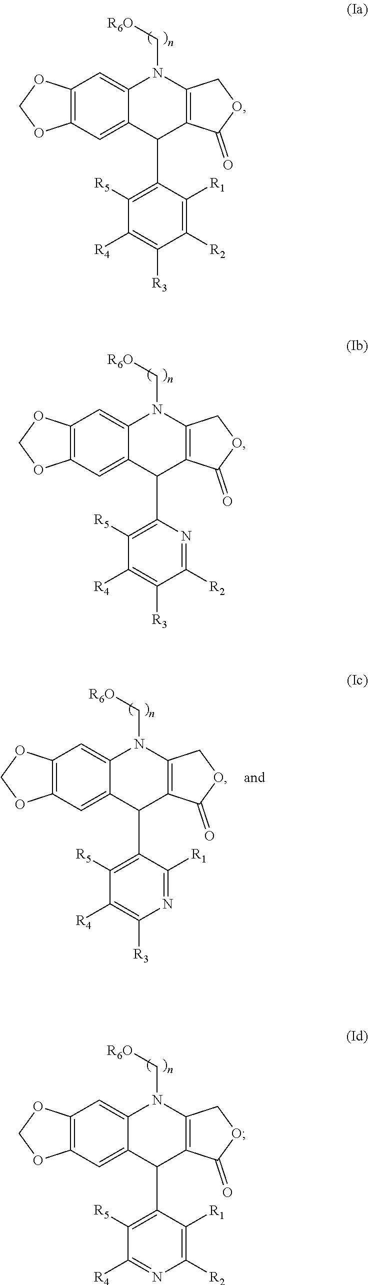

Another embodiment comprises a compound of Formula (I) defined otherwise as above, wherein X, R₁, R₂, R₃, R₄, R₅, and R₆ are as defined and subject to the provisos above, and n is an integer independently in each instance from the group of 1, 2, 3, 4, and 5.

Still another embodiment comprises a compound of Formula (I) defined otherwise as above, wherein X, R₁, R₂, R₃, R₄, R₅, and R₆ are as defined and subject to the provisos above, and n is an integer independently in each instance from the group of 1, 2, 3, and 4.

A further embodiment comprises a compound of Formula (I) defined otherwise as above, wherein X, R₁, R₂, R₃, R₄, R₅, and R₆ are as defined and subject to the provisos above, and n is an integer independently in each instance from the group of 1, 2, and 3.

A further embodiment comprises a compound of Formula (I) defined otherwise as above, wherein X, R₁, R₂, R₃, R₄, R₅, and R₆ are as defined and subject to the provisos above, and n is an integer independently in each instance from the group of 2 and 3.

A further embodiment comprises a compound of Formula (I) defined otherwise as above, wherein X, R₁, R₂, R₃, R₄, R₅, and R₆ are as defined and subject to the provisos above, and n is 2.

A further embodiment comprises a compound of Formula (I) defined otherwise as above, wherein X, R₁, R₂, R₃, R₄, R₅, and R₆ are as defined and subject to the provisos above, and n is 3.

Within each of the embodiments above comprising a compound of Formula (I), there is a further embodiment wherein X, R₁, R₂, R₃, R₄, R₅, and n are as defined for the particular embodiment, wherein R₆ is H.

One embodiment provides a compound of Formula (II):

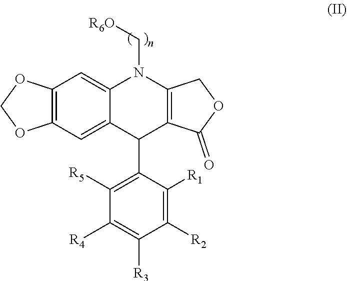

wherein:
R₁, R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH;
n is an integer selected independently in each instance from the group of 1, 2, 3, 4, 5, and 6;
R₆ is selected from the group of H, C₁-C₆ alkyl, C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl; wherein the C₁-C₆ alkyl group is substituted with 0, 1, 2, 3, or 4 substituents selected from F, Cl, Br, I, OH, CN, NO₂, and OH; and the C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl groups are substituted with 0, 1, 2, 3, or 4 substituents selected from the group of C₁-C₃ alkyl, C₁-C₃ alkoxy, F, Cl, Br, I, OH, CN, NO₂, and OH;
with the proviso that at least one of R₁, R₂, R₃, R₄, and R₅ is selected from the group of F, C₁-C₄ fluoroalkyl, and SF₅; and
with the proviso that, when R₂ is F or CF₃, then at least one of R₁, R₃, R₄, and R₅ is not H; or a pharmaceutically acceptable salt thereof.

Another embodiment comprises a compound of Formula (II), wherein R₁, R₂, R₃, R₄, R₅, and R₆ are as defined and subject to the provisos above, and n is an integer independently in each instance from the group of 1, 2, 3, 4, and 5.

Still another embodiment comprises a compound of Formula (II), wherein R₁, R₂, R₃, R₄, R₅, and R₆ are as defined and subject to the provisos above, and n is an integer independently in each instance from the group of 1, 2, 3, and 4.

A further embodiment comprises a compound of Formula (II), wherein R₁, R₂, R₃, R₄, R₅, and R₆ are as defined and subject to the provisos above, and n is an integer independently in each instance from the group of 1, 2, and 3.

A further embodiment comprises a compound of Formula (II), wherein R₁, R₂, R₃, R₄, R₅, and R₆ are as defined and subject to the provisos above, and n is an integer independently in each instance from the group of 2 and 3.

A further embodiment comprises a compound of Formula (II), wherein R₁, R₂, R₃, R₄, R₅, and R₆ are as defined and subject to the provisos above, and n is 2.

A further embodiment comprises a compound of Formula (II), wherein R₁, R₂, R₃, R₄, R₅, and R₆ are as defined and subject to the provisos above, and n is 3.

Within each of the embodiments above comprising a compound of Formula (II), there is a further embodiment wherein R₁, R₂, R₃, R₄, R₅, and n are as defined for the particular embodiment, wherein R₆ is H.

A further embodiment provides a compound of Formula (III),

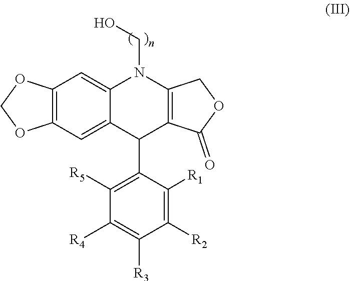

wherein: n is an integer selected independently in each instance from the group of 1, 2, 3, 4, 5, and 6;
R₁, R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH;
with the proviso that at least one of R₁, R₂, R₃, R₄, and R₅ is selected from the group of F, C₁-C₄ fluoroalkyl, and SF₅; and
with the proviso that, when R₂ is F or CF₃, then at least one of R₁, R₃, R₄, and R₅ is not H; or a pharmaceutically acceptable salt thereof.

Another embodiment provides a compound of Formula (III), or a pharmaceutically acceptable salt thereof, wherein n is an integer selected independently in each instance from the group of 1, 2, 3, 4, 5, and 6; R₁ is selected from the group of F, C₁-C₄ fluoroalkyl, and SF₅; and R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH.

Yet another embodiment provides a compound of Formula (III), or a pharmaceutically acceptable salt thereof, wherein n is an integer selected independently in each instance from the group of 1, 2, 3, 4, 5, and 6; R₃ is selected from the group of F, C₁-C₄ fluoroalkyl, and SF₅; and R₁, R₂, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH.

A still further embodiment provides a compound of Formula (III), or a pharmaceutically acceptable salt thereof, wherein:
n is an integer selected independently in each instance from the group of 1, 2, 3, 4, 5, and 6;
R₁, R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are selected from the group of F, C₁-C₄ fluoroalkyl, and SF₅.

A still further embodiment provides a compound of Formula (III), or a pharmaceutically acceptable salt thereof, wherein:
n is an integer selected independently in each instance from the group of 1, 2, 3, 4, 5, and 6;
R₁, R₂, R₃, R₄, and R₅ are each independently selected from the group of H, F, C₁-C₃ fluoroalkyl, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are selected from the group of F, C₁-C₃ fluoroalkyl.

A still further embodiment provides a compound of Formula (III), or a pharmaceutically acceptable salt thereof, wherein:
n is an integer selected independently in each instance from the group of 1, 2, 3, 4, 5, and 6;
R₁, R₂, R₃, R₄, and R₅ are each independently selected from the group of H, F, CF₃, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are selected from the group of F and CF₃.

A still further embodiment provides a compound of Formula (III), or a pharmaceutically acceptable salt thereof, wherein:
n is an integer selected independently in each instance from the group of 1, 2, 3, 4, 5, and 6;
R₁, R₂, R₃, R₄, and R₅ are each independently selected from the group of H, F, CF₃, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are F.

Another embodiment comprises a compound of Formula (III), wherein R₁, R₂, R₃, R₄, and R₅ are as defined and subject to the proviso above, and n is an integer independently in each instance from the group of 1, 2, 3, 4, and 5.

Still another embodiment comprises a compound of Formula (III), wherein R₁, R₂, R₃, R₄, and R₅ are as defined and subject to the proviso above, and n is an integer independently in each instance from the group of 1, 2, 3, and 4.

A further embodiment comprises a compound of Formula (III), wherein R₁, R₂, R₃, R₄, and R₅ are as defined and subject to the proviso above, and n is an integer independently in each instance from the group of 1, 2, and 3.

A further embodiment comprises a compound of Formula (III), wherein R₁, R₂, R₃, R₄, and R₅ are as defined and subject to the proviso above, and n is an integer independently in each instance from the group of 2 and 3.

A further embodiment comprises a compound of Formula (III), wherein R₁, R₂, R₃, R₄, and R₅ are as defined and subject to the proviso above, and n is 2.

A further embodiment comprises a compound of Formula (III), wherein R₁, R₂, R₃, R₄, and R₅ are as defined and subject to the proviso above, and n is 3.

### Pharmaceutical Compositions

Also provided is a pharmaceutical composition comprising a pharmaceutically effective amount of a compound of Formula (I), or a pharmaceutically acceptable salt, pharmaceutically acceptable co-crystal, ester, solvate, hydrate, isomer, tautomer, isotope, polymorph, or prodrug thereof, and a pharmaceutically acceptable carrier or excipient.

### Methods of Treatment

Each of the methods of treatment below will reference the use of a compound of Formula (I), or a pharmaceutically acceptable salt, pharmaceutically acceptable co-crystals, pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs thereof. It is understood that each method provides additional embodiments wherein the compound or compounds used is of Formula (Ia), Formula (Ib), Formula (Ic), Formula (Id), Formula (II), Formula (III), SU056, etc., or a pharmaceutically acceptable salt, pharmaceutically acceptable co-crystal, ester, solvate, hydrate, isomer, tautomer, isotope, polymorph, or prodrug thereof.

Provided is a method of inhibiting YB1 protein activity in a subject, the method comprising administering to the subject in need thereof a pharmaceutically effective amount of a compound of Formula (I), or a pharmaceutically acceptable salt, pharmaceutically acceptable co-crystal, ester, solvate, hydrate, isomer, tautomer, isotope, polymorph, or prodrug thereof.

Provided is a method of inhibiting YB1 protein activity in a subject experiencing a cancer, the method comprising administering to the subject in need thereof a pharmaceutically effective amount of a compound of Formula (I), or a pharmaceutically acceptable salt, pharmaceutically acceptable co-crystal, ester, solvate, hydrate, isomer, tautomer, isotope, polymorph, or prodrug thereof. In different embodiments, the cancer experienced by the subject is selected from the group of a gynecological cancer (including ovarian, endometrial, fallopian tube, and cervical cancers), breast cancers, lung cancers, ovarian cancer, prostate cancer, colorectal cancer, and gastric cancer.

Methods of inhibiting YB1 protein activity may be used in treating other cancers that have been associated with YBX1 expression, including acute myeloid leukemia (Zhou et al., Journal of Experimental & Clinical Cancer Research (2021) 40:353), renal cell carcinoma (Ruan et al., Oncogene (2020) 39:6113-6128), bladder cancer (Xu et al., Oncotarget, 2017, Vol. 8, No. 39, pp. 65946-65956, osteosarcoma (Fujiwara-Okada et al., British Journal of Cancer (2013) 108, pp. 836-847), head and neck cancer (Kolk et al., British Journal of Cancer (2011) 105, pp. 1864-1873, nasopharyngeal carcinoma (Zhou et al., Experimental Cell Research 361 (2017) pp. 126-134 and Ban et al., Journal of Cancer 2021, Vol. 12 (11), pp. 3315-3324).

Also provided is a method of sensitizing cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt, pharmaceutically acceptable co-crystal, ester, solvate, hydrate, isomer, tautomer, isotope, polymorph, or prodrug thereof. It is understood that included herein are separate methods for sensitizing each of the cancers referenced herein to their relevant anticancer agents.

Also provided is a method of sensitizing cancer cells expressing YB1 (YBX1) protein in a subject to treatment with radiation, the method comprising administering to the subject in need thereof a pharmaceutically effective amount of a compound of Formula (I), or a pharmaceutically acceptable salt, pharmaceutically acceptable co-crystal, ester, solvate, hydrate, isomer, tautomer, isotope, polymorph, or prodrug thereof. It is understood that included herein are separate methods for sensitizing each of the cancers referenced herein to radiation therapy.

It is also understood that the pharmaceutically effective amount of a compound of Formula (I), or a pharmaceutically acceptable salt thereof, may be administered in a regimen concurrently with an additional anticancer agent or radiation. In other embodiments, the pharmaceutically effective amount of a compound of Formula (I), or a pharmaceutically acceptable salt thereof, may be administered to a subject in need thereof in a dose or regimen prior to subsequent administration of a designated cancer agent or agents and/or radiation therapy.

In some embodiments, the compound of Formula (I), or a pharmaceutically acceptable salt thereof, may be administered for an initial period of time, such as from 1 to 7 days, followed in sequence by administration to the subject in need thereof of a designated cancer agent or agents and/or radiation therapy.

In further embodiments, the compound of Formula (I), or a pharmaceutically acceptable salt thereof, and one or more designated cancer agent or agents and/or radiation therapy may be administered to the subject in need thereof in repeating sequential periods of time, such as from 1 to 14 days each, with or without a refractory period involving neither treatment in between each pair of administrations.

In other embodiments, the compound of Formula (I) may be administered for an initial period of time, such as from 1 to 7 days, followed by a second period of co-administration to the subject in need thereof of both a pharmaceutically effective amount of a compound of Formula (I), or a pharmaceutically acceptable salt thereof, and a pharmaceutically effective amount of a designated cancer agent or agents and/or radiation therapy.

In different embodiments, the cancer cells expressing YB1 (YBX1) protein in a subject sensitized to the treatments described herein are selected from the group of a gynecological cancer (including ovarian, endometrial, fallopian tube, and cervical cancers), leukemias, lymphomas, kidney cancer, bladder cancer, pancreatic cancer, head and neck cancer, breast cancers (including triple negative, ER-negative, ER-positive breast cancers, and progesterone-positive), lung cancers, ovarian cancer, prostate cancer, colorectal cancer, gastric cancer, and neuronal cancer (including gliomas).

Gynecological Cancers Provided is a method of treatment of gynecological cancers expressing YB1 (YBX1) protein in a subject, the method comprising administering to the subject in need thereof a pharmaceutically effective amount of a compound of Formula (I), or a pharmaceutically acceptable salt thereof.

Also provided is a method of treatment of gynecological cancers expressing YB1 (YBX1) protein in a subject, the method comprising administering to the subject in need thereof a pharmaceutically effective amount of a compound of Formula (I), or a pharmaceutically acceptable salt thereof, and a pharmaceutically effective amount of an mTOR inhibitor, or a pharmaceutically acceptable salt thereof. In some embodiments, the mTOR inhibitor is selected from the group of sirolimus, everolimus, deforolimus, and temsirolimus.

Also provided is a method of enhancing the effect of an anticancer agent in a subject experiencing a gynecological cancer expressing YB1 (YBX1) protein, the method comprising administering to the subject in need thereof a pharmaceutically effective amount of a compound of Formula (I), or a pharmaceutically acceptable salt thereof. In some embodiments, the treatment with a compound of Formula (I) sensitizes gynecological cancer cells expressing YB1 (YBX1) protein in the subject to the treatment of the anticancer agent.

In some embodiments, the anticancer agent used to treat the gynecological cancer is an inhibitor or antagonist of phosphoinositide 3-kinase (PI3K)/protein kinase B (Akt).

In some embodiments, the gynecological cancer expressing YB1 (YBX1) protein to be treated is ovarian cancer. In other embodiments, the gynecological cancer to be treated is endometrial cancer. In still other embodiments, the gynecological cancer to be treated is cervical cancer.

As such, also provided is a method of treatment of ovarian cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of cisplatin, or a pharmaceutically acceptable salt thereof.

As such, also provided is a method of treatment of ovarian cancer expressing YB1 (YBX1) protein in a subject, the method comprising administering to the subject in need thereof:
a pharmaceutically effective amount of a compound of Formula (I), or a pharmaceutically acceptable salt thereof; and
a) a pharmaceutically effective amount of a taxane compound, or a pharmaceutically acceptable salt thereof.

In some embodiments, the taxane compound used in the method of treatment of the gynecological cancers expressing YB1 (YBX1) protein discussed herein is selected from the group of paclitaxel, docetaxel, and cabazitaxel.

For each of the methods of sensitizing cancer cells expressing YB1 (YBX1) protein in a subject to treatment with an anticancer agent and/or radiation or inhibiting YB1 protein activity in a subject experiencing a cancer, there is a corresponding method with the initial step of detecting the presence or absence of expressed YB1 (YBX1) protein in sample cells of the cancer and, when YB1 (YBX1) protein is determined to be present in the sample cells, treating the subject experiencing the cancer in question as described for each method with a pharmaceutically effective amount of a compound of Formula (I), or a pharmaceutically acceptable salt, pharmaceutically acceptable co-crystal, ester, solvate, hydrate, isomer, tautomer, isotope, polymorph, or prodrug thereof, and any other agent or agents indicated by the particular method.

As a non-limiting example, also provided is a method of treatment of ovarian cancer expressing YB1 (YBX1) protein in a subject, the method comprising the steps of:
a) determining the presence or absence of expressed YB1 protein in an ovarian cancer tumor sample collected from the subject in need thereof; and
b) when expressed YB1 protein is determined to be present in the ovarian cancer tumor sample, administering to the subject in need thereof:
   i) a pharmaceutically effective amount of a compound of Formula (I), or a pharmaceutically acceptable salt thereof; and
   ii) a pharmaceutically effective amount of a taxane compound, or a pharmaceutically acceptable salt thereof.

As such, also provided is a method of treatment of fallopian tube cancer (fallopian tube carcinoma) expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a taxane compound, or a pharmaceutically acceptable salt thereof.

As such, also provided is a method of treatment of fallopian tube cancer (fallopian tube carcinoma) expressing YB1 (YBX1) protein in a subject, the method comprising administering to the subject in need thereof:
a) a pharmaceutically effective amount of a compound of Formula (I), or a pharmaceutically acceptable salt thereof;
b) a pharmaceutically effective amount of a taxane compound, or a pharmaceutically acceptable salt thereof; and
c) a pharmaceutically effective amount of carboplatin, or a pharmaceutically acceptable salt thereof.

In some embodiments, the taxane compound in the methods of treating fallopian tube cancer is selected from the group of paclitaxel, albumin-bound paclitaxel, docetaxel, and cabazitaxel.

### Prostate Cancer

Also provided is a method of treatment of prostate cancer expressing YB1 (YBX1) protein in a subject, the method comprising administering to the subject in need thereof a pharmaceutically effective amount of a compound of Formula (I), or a pharmaceutically acceptable salt thereof. Further provided is a method of inhibiting prostate cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing prostate cancer expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

In some embodiments, the administration of a compound of Formula (I) sensitizes the prostate cancer expressing YB1 (YBX1) protein in the subject to treatment with a taxane anticancer agent. In some embodiments, the taxane anticancer agent is selected from the group of paclitaxel, docetaxel, and cabazitaxel, or a pharmaceutically acceptable salt thereof.

As such, also provided is a method of treatment of prostate cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a taxane compound selected from the group of paclitaxel, docetaxel, and cabazitaxel, or a pharmaceutically acceptable salt thereof.

In some embodiments, the administration of a compound of Formula (I) sensitizes the prostate cancer expressing YB1 (YBX1) protein in the subject to treatment with an androgen receptor inhibitor anticancer agent. In some embodiments the androgen receptor inhibitor is selected from the group of apalutamide, enzalutamide, darolutamide, and abiraterone acetate.

As such, also provided is a method of treatment of prostate cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an androgen receptor inhibitor compound selected from the group of apalutamide, enzalutamide, darolutamide, and abiraterone acetate, or a pharmaceutically acceptable salt thereof.

In some embodiments, the apalutamide is administered to the subject in need thereof at a daily dosage of from about 100 mg to about 300 mg. In some embodiments, the apalutamide is administered at a dosage of about 240 mg per day.

In some embodiments, the anticancer agent is a luteinizing hormone-releasing hormone (LHRH) agonist. In some embodiments, the LHRH agonist is selected from the group of leuprolide/leuprorelin, goserelin, triptorelin, buserelin, and histrelin.

As such, also provided is a method of treatment of prostate cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a luteinizing hormone-releasing hormone (LHRH) agonist compound selected from the group of leuprolide/leuprorelin, goserelin, triptorelin, buserelin, and histrelin, or a pharmaceutically acceptable salt thereof.

In other embodiments, the anticancer agent is a luteinizing hormone-releasing hormone (LHRH) antagonist. In some embodiments, the LHRH agonist is degarelix

As such, also provided is a method of treatment of prostate cancer in a subject, the method comprising administering to the subject in need thereof:
a) a pharmaceutically effective amount of a compound of Formula (I), or a pharmaceutically acceptable salt thereof; and
b) a pharmaceutically effective amount of degarelix, or a pharmaceutically acceptable salt thereof.

In other embodiments, the anticancer agent is anti-androgen agent. In some embodiments, the anti-androgen agent is selected from the group of flutamide, bicalutamide, and nilutamide.

As such, also provided is a method of treatment of prostate cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anti-androgen compound selected from the group of flutamide, bicalutamide, and nilutamide, or a pharmaceutically acceptable salt thereof.

In some embodiments concerning the methods herein for treating prostate cancer and/or inhibiting prostate cancer metastasis, the prostate cancer in question is an androgen-independent prostate cancer. In other embodiments, the prostate cancer in question is castration-sensitive prostate cancer. In other embodiments, the prostate cancer in question is metastatic castration-sensitive prostate cancer. In additional embodiments, the prostate cancer expressing YB1 (YBX1) protein to be treated is non-metastatic castration-resistant prostate cancer. In other embodiments, the prostate cancer is hormone-refractory prostate cancer (HRPC).

### Melanoma

Also provided is a method of treatment of melanoma expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting melanoma metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing melanoma cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of melanoma expressing YB1 (YBX1) protein in a subject, the method comprising administering to the subject in need thereof:
c) a pharmaceutically effective amount of a compound of Formula (I), or a pharmaceutically acceptable salt thereof; and
d) a pharmaceutically effective amount of a PD-1 inhibitor agent selected from the group of pembrolizumab and nivolumab, or a pharmaceutically acceptable salt thereof.

Further provided is a method of treatment of melanoma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of atezolizumab, or a pharmaceutically acceptable salt thereof.

Further provided is a method of treatment of melanoma expressing YB1 (YBX1) protein in a subject, the method comprising administering to the subject in need thereof:
a) a pharmaceutically effective amount of a compound of Formula (I), or a pharmaceutically acceptable salt thereof;
b) a pharmaceutically effective amount of atezolizumab, or a pharmaceutically acceptable salt thereof; and
c) a pharmaceutically effective amount of a third agent selected from the group of cobimetinib and vemurafenib, or a pharmaceutically acceptable salt thereof.

Also provided is a method of treatment of melanoma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a CTLA-4 inhibitor (such as ipilimumab).

As such, also provided is a method of treatment of melanoma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of interleukin-2 (IL-2).

### Cisplatin Resistance

YB-1 expression or overexpression has also been associated with resistance to cisplatin treatments in some cancers, including breast, bladder, and ovarian cancers.

In some embodiments, the breast cancer to be treated is refractory to endocrine therapeutics, such as selective estrogen receptor modulators (SERMs), including tamoxifen and toremifene. In some embodiments, the breast cancer is refractory to selective estrogen receptor degrader (SERDs), such as fulvestrant and elacestrant. In other embodiments, the breast cancer to be treated is refractory to aromatase inhibitors, such as letrozole, anastrozole, exemestane, and testolactone.

Provided herein is a method of sensitizing a cancer expressing YB1 (YBX1) protein in a subject to treatment with cisplatin, the method comprising administering to the subject in need thereof a pharmaceutically effective amount of a compound of Formula (I), or a pharmaceutically acceptable salt thereof.

Provided herein is a method of sensitizing a cancer expressing YB1 (YBX1) protein in a subject to treatment with a taxane compound, the method comprising administering to the subject in need thereof a pharmaceutically effective amount of a compound of Formula (I), or a pharmaceutically acceptable salt thereof.

### Breast Cancer

Also provided is a method of treatment of breast cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting breast cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing breast cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof. In some embodiments, the method sensitizes breast cancer cells expressing YB1 (YBX1) protein in the subject in need thereof to treatment with one or more agents selected from the group of anthracyclines (such as doxorubicin, pegylated liposomal doxorubicin, and epirubicin), taxane compounds (such as paclitaxel, albumin-bound paclitaxel, docetaxel, and cabazitaxel), 5-fluorouracil, capecitabine, cyclophosphamide, vinarelbine, gemcitabine, ixabepilone, eribulin, and platinum agents (such as carboplatin and cisplatin). In other embodiments, the method sensitizes breast cancer cells expressing YB1 (YBX1) to treatment with radiation therapy.

An embodiment provides a method of treatment of breast cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of doxorubicin, pegylated liposomal doxorubicin, epirubicin, paclitaxel, docetaxel, 5-fluorouracil, capecitabine, cyclophosphamide, and carboplatin, or a pharmaceutically acceptable salt thereof.

Another embodiment provides a method of treatment of breast cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of paclitaxel, albumin-bound paclitaxel, docetaxel, doxorubicin, pegylated liposomal doxorubicin, epirubicin, cisplatin, carboplatin, vinorelbine, capecitabine, gemcitabine, ixabepilone, and eribulin, or a pharmaceutically acceptable salt thereof.

### Colorectal Cancer

Also provided is a method of treatment of colorectal cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting colorectal cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing colorectal cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of colorectal cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anti-cancer agent selected from the group of 5-fluorouracil, capecitabine, irinotecan, oxaliplatin, and trifluridine and tipiracil, or a pharmaceutically acceptable salt thereof.

### Bladder Cancer

Also provided is a method of treatment of bladder cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting bladder cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing bladder cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

Provided is a method of treatment of bladder cancer expressing YB1 (YBX1) protein in a subject, the method comprising administering to the subject in need thereof:
a) a pharmaceutically effective amount of a compound of Formula (I), or a pharmaceutically acceptable salt thereof;
b) a pharmaceutically effective amount of an anticancer agent selected from the group of cisplatin, cisplatin plus 5-fluorouracil, and mitomycin with 5-fluorouracil, or a pharmaceutically acceptable salt thereof;
c) a therapeutically effective dose of radiation.

Also provided is a method of treatment of bladder cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anticancer agent selected from the group of
   i) gemcitabine and cisplatin;
   ii) Dose-dense methotrexate, vinblastine, doxorubicin (Adriamycin), and cisplatin (DDMVAC);
   iii) Cisplatin, methotrexate, and vinblastine (CMV); and
   iv) Gemcitabine and paclitaxel

Also provided is a method of treatment of bladder cancer expressing YB1 (YBX1) protein in a subject, the method comprising administering to the subject in need thereof:
a) a pharmaceutically effective amount of a compound of Formula (I), or a pharmaceutically acceptable salt thereof;
b) a pharmaceutically effective amount of an anticancer agent selected from the group of docetaxel, paclitaxel, doxorubicin, methotrexate, ifosfamide, and pemetrexed, or a pharmaceutically acceptable salt thereof.

### Liver Cancer

Also provided is a method of treatment of liver cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting liver cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing liver cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of liver cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anti-cancer agent selected from the group of gemcitabine, oxaliplatin, cisplatin, doxorubicin, 5-fluorouracil, capecitabine, and mitoxantrone, or a pharmaceutically acceptable salt thereof.

### Lung Cancer

Also provided is a method of treatment of small cell lung cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting small cell lung cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing small cell lung cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of small cell lung cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anti-cancer agent selected from the group of cisplatin and etoposide, carboplatin and etoposide, cisplatin and irinotecan, and carboplatin and irinotecan, or a pharmaceutically acceptable salt thereof.

Also provided is a method of treatment of non-small cell lung cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting non-small cell lung cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing non-small cell lung cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of non-small cell lung cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anti-cancer agents selected from the group of cisplatin, carboplatin, paclitaxel, albumin-bound paclitaxel, docetaxel, gemcitabine, vinarelbine, etoposide, and pemetrexed, or a pharmaceutically acceptable salt thereof.

### Multiple Myeloma

Also provided is a method of treatment of multiple myeloma expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting multiple myeloma metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing multiple myeloma cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of multiple myeloma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anti-cancer agent selected from the group of Melphalan, vincristine, cyclophosphamide, etoposide, doxorubicin, liposomal doxorubicin, and bendamustine, or a pharmaceutically acceptable salt thereof.

### Soft Tissue Sarcomas

Also provided herein are methods of treatment of soft tissue sarcomas expressing YB1 (YBX1) protein, including angiosarcoma, dermatofibrosarcoma protuberans, epitheloid sarcoma, gastrointestinal stromal tumor (GIST), Kaposi's sarcoma, Leiomyosarcoma, liposarcoma, malignant peripheral nerve sheath tumors, myxofibrosarcoma, rhabdomyosarcoma, solitary fibrous tumors, synovial sarcoma, and undifferentiated pleomorphic sarcoma.

Also provided is a method of treatment of soft tissue sarcomas expressing YB1 (YBX1) protein, such as fibrosarcoma expressing YB1 (YBX1) protein, in a subject, the method comprising administering to the subject in need thereof a pharmaceutically effective amount of a compound of Formula (I), or a pharmaceutically acceptable salt thereof.

Further provided is a method of inhibiting soft tissue sarcomas expressing YB1 (YBX1) protein, such as fibrosarcoma expressing YB1 (YBX1) protein, in a subject, the method comprising administering to the subject in need thereof a pharmaceutically effective amount of a compound of Formula (I), or a pharmaceutically acceptable salt thereof.

Additionally provided is a method of sensitizing soft tissue sarcoma cells expressing YB1 (YBX1) protein, such as fibrosarcoma cells expressing YB1 (YBX1) protein, cells in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of soft tissue sarcomas expressing YB1 (YBX1) protein, such as fibrosarcoma expressing YB1 (YBX1) protein, in a subject, the method comprising administering to the subject in need thereof:
a) a pharmaceutically effective amount of a compound of Formula (I), or a pharmaceutically acceptable salt thereof; and
b) a pharmaceutically effective amount of one or more anti-cancer agents selected from the group of ifosfamide, doxorubicin, dacarbazine (DTIC), epirubicin, temozolomide, docetaxel, gemcitabine, vinorelbine, trabectedin, and eribulin, or a pharmaceutically acceptable salt thereof.

In some embodiments, when the anti-cancer agent ifosfamide is used, the drug mesna is also given to protect the bladder from the toxic effects of ifosfamide. In other embodiments, the anti-cancer agent is a combination of mesna, Adriamycin [doxorubicin], ifosfamide, and dacarbazine, sometimes referred to by the acronym MAID. In other embodiments, the anti-cancer agent is a combination of Adriamycin [doxorubicin], ifosfamide, and mesna, sometimes referred to by the acronym AIM. In some embodiments in the methods of treating soft tissue sarcomas herein, the anti-cancer agent or agents are administered to the subject in need thereof using isolated limb perfusion.

Osteosarcomas: Also provided is a method of treatment of osteosarcoma expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting osteosarcoma expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing osteosarcoma cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of osteosarcoma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anti-cancer agents selected from the group of methotrexate, doxorubicin, cisplatin, carboplatin, Ifosfamide, cyclophosphamide, etoposide, and gemcitabine, or a pharmaceutically acceptable salt thereof.

In some embodiments in the method of treating osteosarcoma expressing YB1 (YBX1) protein above, the anti-cancer agent is a combination of High-dose methotrexate, doxorubicin, and cisplatin (the MAP regimen). In other embodiments, a combination of doxorubicin and cisplatin are administered. In other embodiments, a combination of ifosfamide and etoposide are used. In still other embodiments, a combination is administered of ifosfamide and epirubicin with either cisplatin or carboplatin.

### Ewing's Sarcoma

Also provided is a method of treatment of Ewing's sarcoma expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting Ewing's sarcoma expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing Ewing's sarcoma cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of Ewing's sarcoma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anti-cancer agents selected from the group of cyclophosphamide, doxorubicin, etoposide, Ifosfamide, and vincristine, or a pharmaceutically acceptable salt thereof.

In some embodiments in the method of treating Ewing's sarcoma expressing YB1 (YBX1) protein above, the anti-cancer agent is a combination of vincristine, doxorubicin, and cyclophosphamide, alternating with ifosfamide and etoposide, the regimen referred to as VDC/IE.

### Gastric Cancers

Also provided is a method of treatment of gastric (stomach) cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting gastric cancer expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing gastric cancer cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of gastric cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anti-cancer agents selected from the group of 5-fluorouracil, capecitabine, carboplatin, cisplatin, docetaxel, epirubicin, irinotecan, oxaliplatin, paclitaxel, and trifluridine+tipracil (LONSURF®) or a pharmaceutically acceptable salt thereof.

In some embodiments in the method of treating gastric cancer expressing YB1 (YBX1) protein above, the anti-cancer agent is a combination of epirubicin, cisplatin, and 5-fluorouracil, sometimes referred to by the acronym ECF. In other embodiments, the combination of docetaxel or paclitaxel with either 5-FU or capecitabine, sometimes combined with radiation. In another embodiment cisplatin is administered with either 5-FU or capecitabine, sometimes combined with radiation. In further embodiments paclitaxel and carboplatin are administered, sometimes combined with radiation. In other embodiments the combination of docetaxel, cisplatin, and 5-fluoruracil (DCF) are administered. In others, irinotecan is administered along with cisplatin, 5-flourouracil, or capecitabine. In still others, oxaliplatin is administered with 5-fluorouracil or capecitabine. In still others, trifluridine+tipracil (LONSURF®) is given.

### Glioblastoma

Also provided is a method of treatment of glioblastoma multiforme (GBM or glioblastoma) expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting glioblastoma expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing glioblastoma cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

As such, also provided is a method of treatment of glioblastoma expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anti-cancer agents selected from the group of temozolomide, bevacizumab, lomustine, carmustine, fluzoparil, pembrolizumab, nivolumab, ipilimumab, anlotinib, glasdegib, and bavituximab, or a pharmaceutically acceptable salt thereof.

In some embodiments, the glioblastoma in the methods above is a pediatric glioblastoma expressing YB1 (YBX1) protein. In some embodiments the glioblastoma is a primary glioblastoma expressing YB1 (YBX1) protein. In others, it is a secondary glioblastoma expressing YB1 (YBX1) protein.

### Head and Neck Cancer

Reference to “head and neck cancer” herein refers to any of the cancers of the oral cavity, throat (pharynx, including the nasopharynx, oropharynx, and hypopharynx), larynx, paranasal sinuses, nasal cavity, and salivary glands. The head and neck cancers include Hypopharyngeal cancer, laryngeal cancer, lip and oral cavity cancer, metastatic squamous neck cancer, nasopharyngeal cancer, oropharyngeal cancer, paranasal sinus and nasal cavity cancer, and salivary gland cancer. It is understood that, for each of the methods of treatment of head and neck cancer described herein, disclosed also is the corresponding method for each of the head and neck cancers listed in this paragraph.

Also provided is a method of treatment of head and neck cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting head and neck cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing head and neck cancer expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

In some embodiments concerning the treatment of head and neck cancer expressing YB1 (YBX1), the anticancer agent used to treat the subject is radiation therapy. In some embodiments, the radiation utilized is external-beam radiation therapy. In some embodiments, the treatment comprises administering a pharmaceutically effective amount of one or more EGFR inhibitors to the subject in need thereof. Other embodiments concern respectively administering a pharmaceutically effective amount of larotrectinib (Vitrakvi) and/or larotrectinib to the subject in need thereof.

Other methods of treating head and neck cancer comprise the use of immunotherapy, such as the administration a pharmaceutically effective amount of pembrolizumab and/or nivolumab to the subject in need thereof

Provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anticancer agent selected from the group of paclitaxel, docetaxel, cisplatin, carboplatin, 5-fluorouracil, methotrexate, and capecitabine, or a pharmaceutically acceptable salt thereof.

As such, also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a taxane compound selected from the group of paclitaxel and docetaxel, or a pharmaceutically acceptable salt thereof.

Also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a cisplatin, or a pharmaceutically acceptable salt thereof.

In some embodiments, the cisplatin is administered to the subject in need thereof at a dose of from about 20 mg/m² to about 100 mg/m² delivered every 3 weeks×3.

Also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of a carboplatin, or a pharmaceutically acceptable salt thereof; and
c) a pharmaceutically effective amount of a drug selected from the group of 5-fluorouracil (5FU) and cetuximab.

Also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of cisplatin, or a pharmaceutically acceptable salt thereof; and
c) a pharmaceutically effective amount of a drug selected from the group of 5-fluorouracil (5FU) and cetuximab.

Also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of cisplatin, or a pharmaceutically acceptable salt thereof; and
c) a pharmaceutically effective amount of paclitaxel, or a pharmaceutically acceptable salt thereof.

Also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of carboplatin, or a pharmaceutically acceptable salt thereof; and
c) a pharmaceutically effective amount of paclitaxel, or a pharmaceutically acceptable salt thereof.

Also provided is a method of treatment of head and neck cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of hydroxyurea, or a pharmaceutically acceptable salt thereof; and
c) a pharmaceutically effective amount of a drug selected from the group of 5-fluorouracil (5FU) and cetuximab.

Further provided is a method of treatment of nasopharyngeal cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of hydroxyurea, or a pharmaceutically acceptable salt thereof; and
c) a pharmaceutically effective amount of a drug selected from the group of carboplatin, doxorubicin, epirubicin, paclitaxel, docetaxel, gemcitabine, bleomycin, and methotrexate.

### Pancreatic Cancer

Provided is a method of treatment of pancreatic cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting pancreatic cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing pancreatic cancer expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

Provided is a method of treatment of pancreatic cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of gemcitabine, 5-fluorouracil, oxaliplatin, paclitaxel, albumin-bound paclitaxel, docetaxel, capecitabine, cisplatin, and irinotecan, or a pharmaceutically acceptable salt thereof.

### Neuronal Cancers

The compounds of Formula (I) may also be used to treat and/or sensitize to treatment neuronal cancers (brain and spinal cord cancers), including medulloblastoma, glioblastoma multiforme (GBM), astrocytomas (anaplastic astrocytomas and pilocytic astrocytomas), ependymomas, and oligodendrogliomas. It is understood for each of the following methods for treating a neuronal cancer or sensitizing a neuronal cancer to treatment described herein, included separate methods of each type for each of the neuronal cancers listed in this paragraph.

Provided is a method of treatment of neuronal cancer expressing YB1 (YBX1) protein in a subject, the method comprising administering to the subject in need thereof a pharmaceutically effective amount of a compound of Formula (I), or a pharmaceutically acceptable salt thereof.

Further provided is a method of inhibiting neuronal cancer metastasis expressing YB1 (YBX1) protein in a subject, the method comprising administering to the subject in need thereof a pharmaceutically effective amount of a compound of Formula (I), or a pharmaceutically acceptable salt thereof.

Additionally provided is a method of sensitizing neuronal cancer expressing YB1 (YBX1) protein in a subject to treatment with an anticancer agent, the method comprising administering to the subject in need thereof a pharmaceutically effective amount of a compound of Formula (I), or a pharmaceutically acceptable salt thereof.

Provided is a method of treatment of neuronal cancer expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of carboplatin, carmustine (BCNU), cisplatin, irinotecan, cyclophosphamide, etoposide, lomustine, methotrexate, procarbazine, temozolomide, and vincristine, or a pharmaceutically acceptable salt thereof.

In some embodiments, the pharmaceutically effective amount of carmustine administered to the subject in need thereof is in the form of a carmustine wafer or implant, such as that in the GLIADEL® Wafer (carmustine implant) product available from Arbor Pharmaceuticals, LLC.

### Leukemias

Methods of the present invention also include those for the treatment of leukemias, wherein the leukemia cells in question express YB-1 protein, including acute myeloid leukemia (AML), Chronic myelogenous leukemia (CML), acute lymphoblastic (or lymphocytic) leukemia (ALL), and chronic lymphocytic leukemia (CLL). It is understood that for each method described herein for a treatment for leukemia or of sensitizing a leukemia to a treatment, separate corresponding methods are understood for each of the referenced leukemias (AML, CML, ALL, and CLL).

Additionally provided is a method of sensitizing leukemia cells expressing YB1 (YBX1) protein in a subject to treatment with an anticancer agent, the method comprising administering to the subject in need thereof a pharmaceutically effective amount of a compound of Formula (I), or a pharmaceutically acceptable salt thereof. In some embodiments, the anticancer agent used to treat the subject is radiation therapy.

Also provided is a method of treatment of acute myeloid leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of an anthracycline drug selected from the group of daunorubicin and idarubicin, or a pharmaceutically acceptable salt thereof; and
c) a pharmaceutically effective amount of cytarabine, or a pharmaceutically acceptable salt thereof

Also provided is a method of treatment of acute myeloid leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of cladribine (2-CdA), fludarabine, mitoxantrone, etoposide, 6-thioguanine, hydroxyurea, prednisone, dexamethasone, methotrexate, 6-mercaptopurine, azacitidine, and decitabine, or a pharmaceutically acceptable salt thereof.

Also provided is a method of treatment of chronic myeloid leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of hydroxyurea, cytarabine (Ara-C), busulfan, cyclophosphamide (CYTOXAN®), and vincristine (ONCOVIN®), or a pharmaceutically acceptable salt thereof

Also provided is a method of treatment of chronic myeloid leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more tyrosine kinase inhibitor anticancer agents selected from the group of imatinib (GLEEVEC®), dasatinib (SPRYCEL®), nilotinib (TASIGNA®), bosutinib (BOSULIF®), ponatinib (ICLUSIG®), and asciminib (SCEMBLIX®), or a pharmaceutically acceptable salt thereof.

Also provided is a method of treatment of chronic myeloid leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of interferon-alpha, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of vincristine, dexamethasone, imatinib, prednisone, doxorubicin and daunorubicin, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of methotrexate, 6-mercaptopurine, vincristine, prednisone, and imatinib, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of vincristine, dexamethasone, prednisone, doxorubicin, and daunorubicin, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of methotrexate, 6-mercaptopurine (6-MP), vincristine, prednisone, and imatinib, or a pharmaceutically acceptable salt thereof.

Also provided is a method of treatment of chronic lymphocytic leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of one or more anticancer agents selected from the group of ibrutinib, acalabrutinib, idelalisib, and duvelisib, or a pharmaceutically acceptable salt thereof.

Also provided is a method of treatment of chronic lymphocytic leukemia expressing YB1 (YBX1) protein in a subject, the method comprising administering to the subject in need thereof:
a) a pharmaceutically effective amount of a compound of Formula (I), or a pharmaceutically acceptable salt thereof; and
b) a pharmaceutically effective amount of venetoclax, or a pharmaceutically acceptable salt thereof.

Also provided is a method of treatment of chronic lymphocytic leukemia expressing YB1 (YBX1) protein in a subject, the method comprising administering to the subject in need thereof:
a) a pharmaceutically effective amount of a compound of Formula (I), or a pharmaceutically acceptable salt thereof; and
c) a pharmaceutically effective amount of one or more anticancer agents selected from the group of rituximab, ofatumumab, and obinutuzumab, or a pharmaceutically acceptable salt thereof.

### Definitions

The terms “YB1” and “YBX1” refer to Y box binding protein 1, also known as Y-box transcription factor or nuclease-sensitive element-binding protein 1, a protein that in humans is encoded by the YBX1 gene.

The wavy line ( [symbol] ) in chemical structures indicates a bond through which the structure shown is bound to another chemical moiety or group.

A “heterocycle” or “heterocyclic group” herein refers to a chemical ring containing carbon atoms and at least one ring heteroatom selected from O, S, and N. The terms “heterocycle” and “heterocyclic” include groups with saturated rings, partially unsaturated rings, and aromatic rings (i.e., heteroaromatic rings). Examples of 5-membered and 6-membered heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, 4-piperidinyl, pyrrolidinyl, 2-pyrrolidonyl, pyrrolinyl, tetrahydrofuranyl, triazinyl, 6H-1,2,5-thiadiazinyl, 2H,6H-1,5,2-dithiazinyl, thienyl, thianthrenyl, pyranyl, 2H-pyrrolyl, isothiazolyl, isoxazolyl, pyrazinyl, pyridazinyl, imidazolidinyl, imidazolinyl, pyrazolidinyl, pyrazolinyl, piperazinyl, morpholinyl, and oxazolidinyl.

The term “alkyl” refers to a straight or branched hydrocarbon. For example, an alkyl group can include those having 1 to 6 carbon atoms (i.e, C₁-C₆ alkyl), 1 to 4 carbon atoms (i.e., C₁-C₄ alkyl), or 1 to 3 carbon atoms (i.e., C₁-C₃ alkyl). Examples of suitable alkyl groups include, but are not limited to, methyl, ethyl, n-propyl, isopropyl (—CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and the like.

The term “alkoxy” refers to a group having the formula —O-alkyl, in which an alkyl group, as defined above, is attached to the parent molecule via an oxygen atom. The alkyl portion of an alkoxy group can have 1 to 6 carbon atoms (i.e., C₁-C₆ alkoxy), 1 to 4 carbon atoms (i.e., C₁-C₄ alkoxy), or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), n-propoxy (—CH₂—CH₂—CH₃), isopropoxy (—CH(CH₃)₂), n-butyl (—CH₂—CH₂—CH₂—CH₃), isobutoxy (—CH₂—CH(CH₃)₂), sec-butoxy (—CH(CH₃)CH₂—CH₃), t-butoxy (—O—C(CH₃)₃ or —OtBu), and the like.

The term “cycloalkyl” refers to a saturated ring having 3 to 6 carbon atoms as a monocycle, including cyclopropyl, cyclobutyl, cyclopentyl, and cyclohexyl groups.

The term “subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula (I), Formula (II), or Formula (III), or a pharmaceutically acceptable salt or co-crystal thereof, as described herein. This includes a subject who may be determined to be at risk of or susceptible to such diseases or conditions, such that treatment would prevent the disease or condition from developing.

In some embodiments, a “subject in need thereof” concerning a method of treatment herein is a patient from whom a tumor sample, such as from a tumor biopsy, is taken and the presence of expressed YBX1 protein is identified in the sampled material, such as through immunohistochemical or Western Blotting techniques known in the art.

The terms “effective amount,” “therapeutically effective amount,” or “pharmaceutically effective amount” refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, an “effective amount,” “therapeutically effective amount,” or a “pharmaceutically effective amount” of a compound of Formula (I), Formula (II), or Formula (III), or a pharmaceutically acceptable salt or co-crystal thereof, is an amount sufficient to modulate YXB1 expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of YXB1 activity.

In some embodiments, an “effective amount” is an amount of a subject compound that, when administered to an individual in one or more doses, in monotherapy or in combination therapy, is effective to inhibit YB-1 by about 20% (20% inhibition), at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the YB-1 activity in the individual in the absence of treatment with the compound, or alternatively, compared to the YB-1 activity in the individual before or after treatment with the compound.

In some embodiments, an “effective amount” is an amount of a subject compound that, when administered to an individual in one or more doses, in monotherapy or in combination therapy, is effective to decrease tumor burden in the subject by about 20%, at least about 30%, at least about 40%, at least about 50%, at least about 60%, at least about 70%, at least about 80%, or at least about 90%, compared to tumor burden in the individual in the absence of treatment with the compound, or alternatively, compared to the tumor burden in the subject before or after treatment with the compound. As used herein the term “tumor burden” refers to the total mass of tumor tissue carried by a subject with cancer. In some embodiments, an “effective amount” is an amount of a subject compound that, when administered to an individual in one or more doses, in monotherapy or in combination therapy, is effective to reduce the dose of radiotherapy required to observe tumor shrinkage in the subject by about 20%, at least about 30%, at least about 40%, at least about 50%, at least about 60%, at least about 70%, at least about 80%, or at least about 90%, compared to the dose of radiotherapy required to observe tumor shrinkage in the individual in the absence of treatment with the compound.

In some embodiments, an “effective amount” of a compound is an amount that, when administered in one or more doses to an individual having cancer, is effective to achieve a 1.5-log, a 2-log, a 2.5-log, a 3-log, a 3.5-log, a 4-log, a 4.5-log, or a 5-log reduction in tumor size.

In some embodiments, a compound of Formula (I), or a pharmaceutically acceptable salt thereof, may be administered at a daily dose of from about 0.2 mg/kg to about 10 mg/kg. In other embodiments, it may be administered at a daily dose of from about 0.2 mg/kg to about 5 mg/kg.

In some embodiments, an effective amount of a compound is an amount that ranges from about 50 ng/kg body weight to about 50 pg/kg body weight (e.g., from about 50 ng/kg body weight to about 40 pg/kg body weight, from about 30 ng/kg body weight to about 20 pg/kg body weight, from about 50 ng/kg body weight to about 10 pg/kg body weight, from about 50 ng/kg body weight to about 1 pg/kg body weight, from about 50 ng/kg body weight to about 800 ng/kg body weight, from about 50 ng/kg body weight to about 700 ng/kg body weight, from about 50 ng/kg body weight to about 600 ng/kg body weight, from about 50 ng/kg body weight to about 500 ng/kg body weight, from about 50 ng/kg body weight to about 400 ng/kg body weight, from about 60 ng/kg body weight to about 400 ng/kg body weight, from about 70 ng/kg body weight to about 300 ng/kg body weight, from about 60 ng/kg body weight to about 100 ng/kg body weight, from about 65 ng/kg body weight to about 85 ng/kg body weight, from about 70 ng/kg body weight to about 90 ng/kg body weight, from about 200 ng/kg body weight to about 900 ng/kg body weight, from about 200 ng/kg body weight to about 800 ng/kg body weight, from about 200 ng/kg body weight to about 700 ng/kg body weight, from about 200 ng/kg body weight to about 600 ng/kg body weight, from about 200 ng/kg body weight to about 500 ng/kg body weight, from about 200 ng/kg body weight to about 400 ng/kg body weight, or from about 200 ng/kg body weight to about 300 ng/kg body weight).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 pg, from about 1 pg to about 10 pg, from about 10 pg to about 50 pg, from about 50 mg to about 150 gg, from about 150 gg to about 250 gg, from about 250 gg to about 500 gg, from about 500 gg to about 750 gg, from about 750 gg to about 1 g, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Administration of an effective amount of a subject compound to an individual with cancer can result in one or more of: 1) a reduction in tumor burden; 2) a reduction in the dose of radiotherapy required to effect tumor shrinkage (e.g. resulting from sensitization to radiotherapy); 3) a reduction in the spread of a cancer from one cell to another cell in an individual; 4) a reduction of morbidity or mortality in clinical outcomes; 5) shortening the total length of treatment when combined with other anti-cancer agents (e.g. resulting from sensitization to other anti-cancer agents); and 6) an improvement in an indicator of disease response (e.g., a reduction in one or more symptoms of cancer). Any of a variety of methods can be used to determine whether a treatment method is effective. For example, a biological sample obtained from an individual who has been treated with a subject method can be assayed.

The terms “inhibiting” or “inhibition” indicates a decrease, such as a significant decrease, in the baseline activity of a biological activity or process. “Inhibition of YB-1 activity” refers to a decrease in YB-1 activity as a direct or indirect response to the presence of a compound of Formula I, or a pharmaceutically acceptable salt or co-crystal thereof, relative to the activity of YB-1 in the absence of such compound or a pharmaceutically acceptable salt or co-crystal thereof. The decrease in activity may be due to the direct interaction of the compound with YB-1, or due to the interaction of the compound(s) described herein with one or more other factors that in turn affect YB-1 activity. For example, the presence of the compound(s) may decrease YB-1 activity by directly binding to the YB-1, by causing (directly or indirectly) another factor to decrease YB-1 activity, or by (directly or indirectly) decreasing the amount of YB-1 present in the cell or organism. In some embodiments, the inhibition of YB-1 activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibition activity desired.

The term “pharmaceutical composition” refers to a composition containing a pharmaceutically effective amount of one or more of the isotopic compounds described herein, or a pharmaceutically acceptable salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

As used herein, “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable carrier” refers to any ingredient in a pharmaceutical composition other than the disclosed pharmaceutically active or therapeutic compounds, or a pharmaceutically acceptable salt thereof (e.g., a carrier capable of suspending or dissolving the active isotopic compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt” includes, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

Also described for a compound of Formula I are the pharmaceutically acceptable salts, pharmaceutically acceptable co-crystals, pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs of such compounds. For the sake of brevity, the list of forms in the prior sentence may not be listed in all references to compounds herein, including those of Formula (I), Formula (Ia), Formula (Ib), Formula (Ic), Formula (Id), Formula (II), Formula (III), SU056, etc., but each is understood to be disclosed and included herein, even if only pharmaceutically acceptable salts are included in a description applied anywhere herein, including in association with descriptions of chemical compounds, pharmaceutical compositions, methods of use/treatment, or other references.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The term “refractory” used herein in regard to a cancer refers to a cancer that does not respond to one or more treatments. In some embodiments, the cancer does not respond to one or more chemotherapeutic agents. In other embodiments, the cancer does not respond to radiation therapy. In other embodiments, the refractory cancer does not respond to one or more chemotherapeutic agents and radiation therapy. A refractory cancer may be resistant at the beginning of such treatments or may become resistant over the course of one or more treatments. In cases of chemotherapy, refractory cancers may also be referred to as “chemotherapy resistant cancers” or “chemo-resistant cancers.”

“Androgen-independent prostate cancer” or “hormone-refractory prostate cancer (AIPC)” is prostate cancer that progresses after primary androgen-ablation therapy, either from orchiectomy or a gonadotropin-releasing hormone (LHRH) agonist, followed by addition and subsequent withdrawal of an antiandrogen. In some embodiments hormone-refractory prostate cancer is defined as 2-3 consecutive rises in prostate-specific antigen (PSA) levels obtained at intervals of greater than 2 weeks and/or documented disease progression based on findings from CT scan and/or bone scan, bone pain, or obstructive voiding symptoms. In some embodiments, the PSA level does not rise at diagnosis or throughout the entire course of the disease. In some embodiments, the prostate cancer is an advanced prostate cancer. In some embodiments, the prostate cancer or metastatic prostate cancer is resistant to treatments with hormone-blocking therapies, such as abiraterone (ZYTIGA®), enzalutamide (XTANDI®), bicalutamide (CASODEX®), flutamide (DROGENIL®), or cyproterone acetate (CYPROSTAT®).

Cancer cells that are resistant to radiation treatment, whether intrinsically or acquired over time, are referred to as “radiation resistant cancers” or “radioresistant cancers.” The terms are intended to describe cancer cells that are less responsive to radiation treatments than non-resistant cancer cells.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

By “significant” is meant any detectable change that is statistically significant in a standard parametric test of statistical significance such as Student's T-test, where p<0.05.

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

The descriptions herein set forth exemplary methods, parameters and the like. It should be recognized, however, that such descriptions are not intended as a limitation on the scope of the present disclosure but is instead provided as a description of exemplary embodiments.

### Results

### Drug Design and Synthesis

We previously discovered an effective, azopodophyllotoxin-based small molecule that blocks GBP1:PIM1 activity in vitro, 9-(3-fluorophenyl)-4-(2-hydroxyethyl)-6-methoxy-4,9-dihydrofuro[3,4-b]quinolin-1(3H)-one, since dubbed SU093 (Andreoli et al., J Med Chem, 57, 7916-32, 2014). This study revealed that SU093 was more active in cell lines that were more resistant to microtubule-targeting agents, such as paclitaxel. Motivated by this work, we have now designed a SU093 analog incorporating fluorine (F) bioisostere group to develop an optimized lead compound SU056 with improved inhibitory properties capable of reducing OC disease progression and sensitizing to OC chemotherapy.

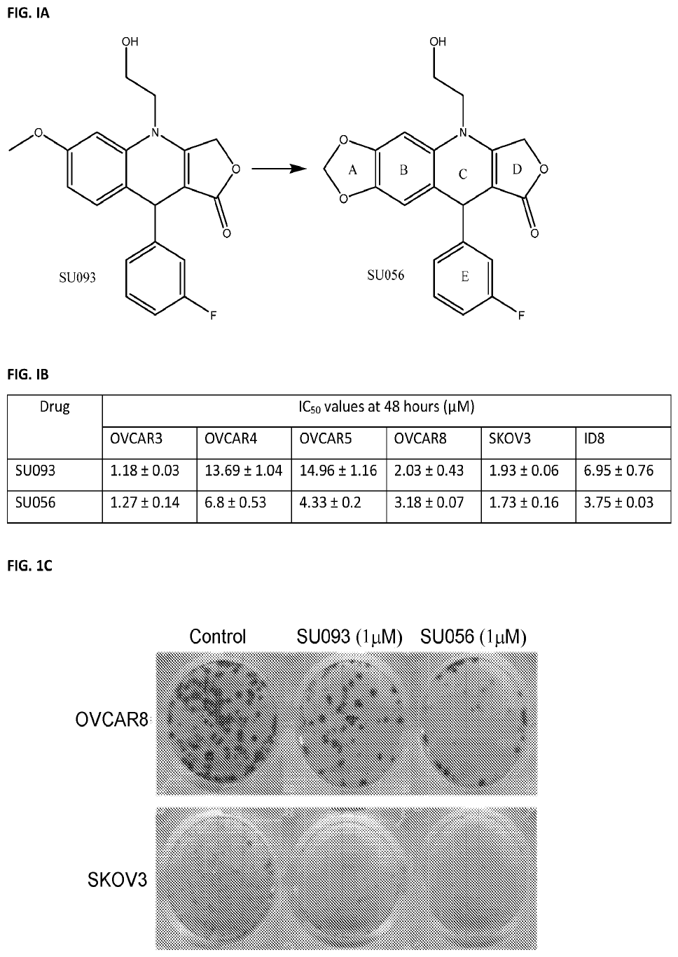

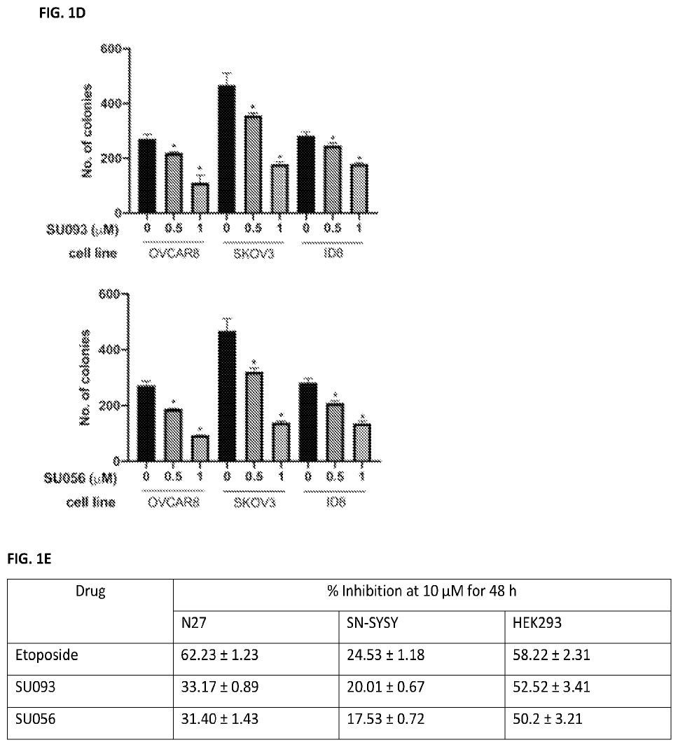

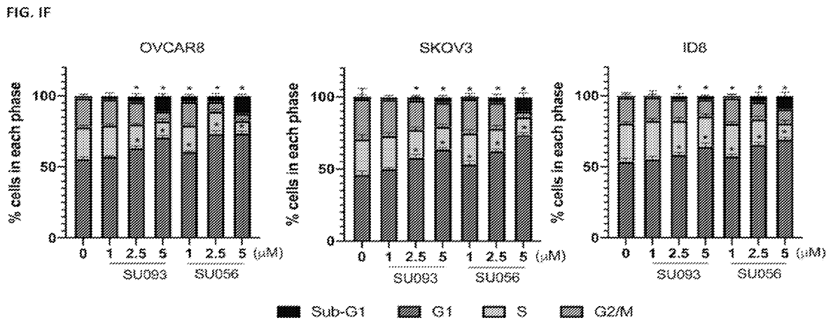

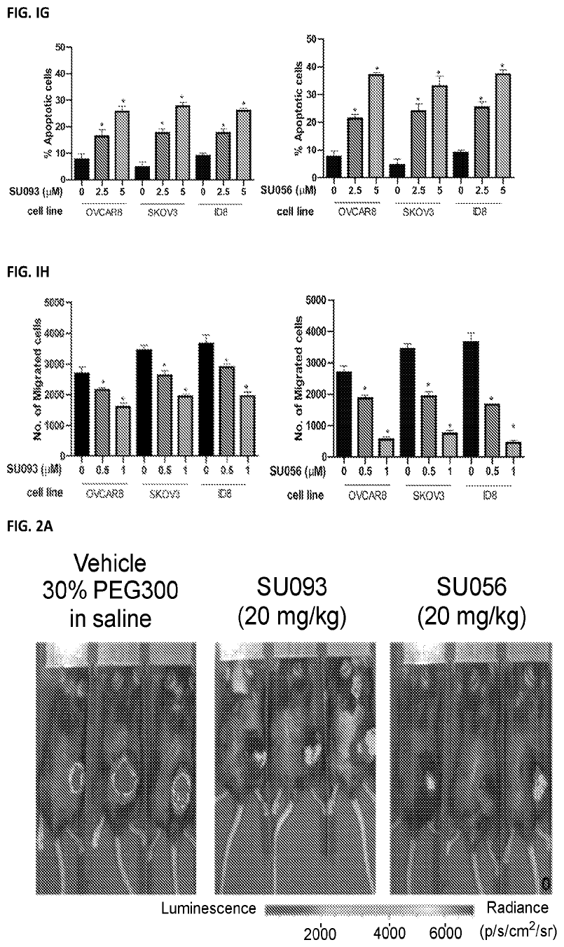

The introduction and manipulation of bioisostere groups offer several advantages in the drug design and development process, such as enhancing the desired biological or physical properties, reducing toxicity, and even altering the metabolism of a given drug compound. Fluorine, considered a classical bioisostere group for hydrogen, has been studied extensively since the first approval of a fluorine-containing drug (9α-fluorocortisol) in 1955 (Fried and Sabo, 1954). Over the years several strategies have been developed to introduce fluorine in drug design. Incorporation of fluorine can influence the lipophilicity of the drug due to the introduction of a strong dipole moment that is closely aligned with carbonyl groups and, compared to that of hydrogen, increases the van der Waals radii of the active sites (Gillis et al., 2015). Direct replacement of hydrogen or even methyl (CH₃) groups in known drugs could lead to improved potency due to the alteration of substrate susceptibility to intracellular oxidative metabolism. This has been seen in the case of paclitaxel, where the substitution of two methyl groups for fluorine in the taxol derivative (3′-difluorovinyl taxoid) enhanced the potency by at least 1,000-fold (Kuznetsova et al., 2012). Additionally, this difluoro-taxol derivative was resistant to metabolic modification by CYP 450 enzymes. Based on these observations, we modified our previously reported compound SU093 by introducing Fluorine as a bioisotere group in the ring E to obtain a new AzP derivative SU056 (FIG. 1A), which was synthesized using a multicomponent reaction similar to SU093 (Andreoli et al., 2014) as described in supplementary information.

### AzP Treatment Inhibits Ovarian Cancer Cell Proliferation

AzP analogs were screened against an ovarian cancer cell line panel (OVCAR-3, OVCAR-4, OVCAR-5, OVCAR-8, and SKOV-3) with an additional screening on HEK293, SH-SY5Y, and N27 cell lines. We tested the relationship between AzPs dose and the viability of human ovarian cancer cell lines and the ID8 murine ovarian cancer cell line. As shown in FIG. 1B, SU056 showed decreased IC₅₀ values after 48 h of treatment compared to SU093 (In OVCAR4, OVCAR5 and ID8 cells, this decrease was up to 2 fold), a trend which was also reflected in the clonogenic assay shown in FIGS. 1C and 1D, where both inhibitors decreased OC colony formation in a dose-dependent and significant manner in OVCAR-8 and ID8 cells. On the other hand, SU093 moderately affect SKOV-3 at 0.5 & 1 μM concentrations.

Etoposide is natural podophyllotoxin and an approved chemotherapy drug used to treat many cancers. A major limitation of etoposide is the potential for neuropathy and neurotoxicity in long-term treatment. We tested the cytotoxic effect of etoposide, SU093, and SU056 on SH-SYSY and N27 neuronal cell lines at 10 μM dose for 48 h to compare neurotoxicity profiles. Cell viability assayed by MTT found that SU056 was markedly less cytotoxic at 49.54% (N27) and 28.51% (SH-SY5Y) less toxic than etoposide (FIG. 1E). These results suggest that bioisostere modification improved overall efficacy in inhibiting OC proliferation with reduced neurotoxic side effects.

### AzP Treatment Causes Cell Cycle Arrest, Apoptosis, and Cell Migration Inhibition

To determine whether proliferation inhibition was the dominant factor driving the reduction in OC viability seen with AzP treatment, we used propidium iodide and annexin-FITC stains with flow cytometry to measure the distribution of cell cycle phases after treatment. Data showed a significant, dose-dependent arrest of OC lines in the sub-G1 and G1 phases and concomitant decreases in the proportion of cells in the S or G2/M phases of the cell cycle (FIG. 1F). SU056 demonstrated greater cell cycle arrest than SU093. This trend was again mirrored in the proportion of apoptotic cells in each treated culture, where AzP treatment significantly increased apoptosis at all doses and in each OC line studied (FIG. 1G). Furthermore, in the Boyden chamber assay for cell migration, SU093 inhibited cell migration by 40-46% (1 μM, P<0.05) in different OC cell lines. However, SU056 treatment was the most effective as it caused 78-87% (1 μM, P<0.05) inhibition in the same cell lines (FIG. 1H). These data show that SU056 treatment-mediates cell cycle arrest, significantly decreases proliferation and migration.

### AzP Inhibits Tumor Progression & Metastasis In Vivo

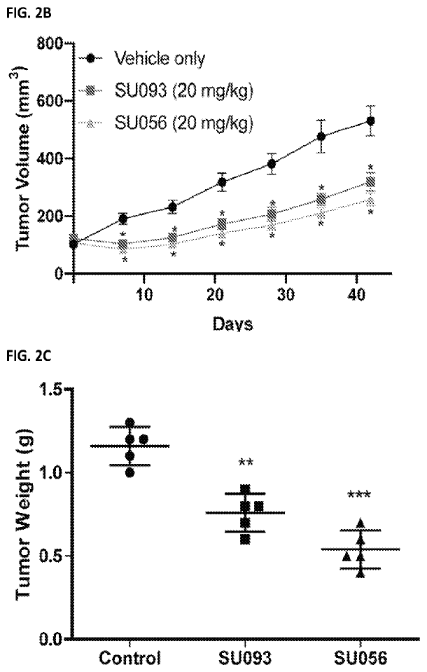

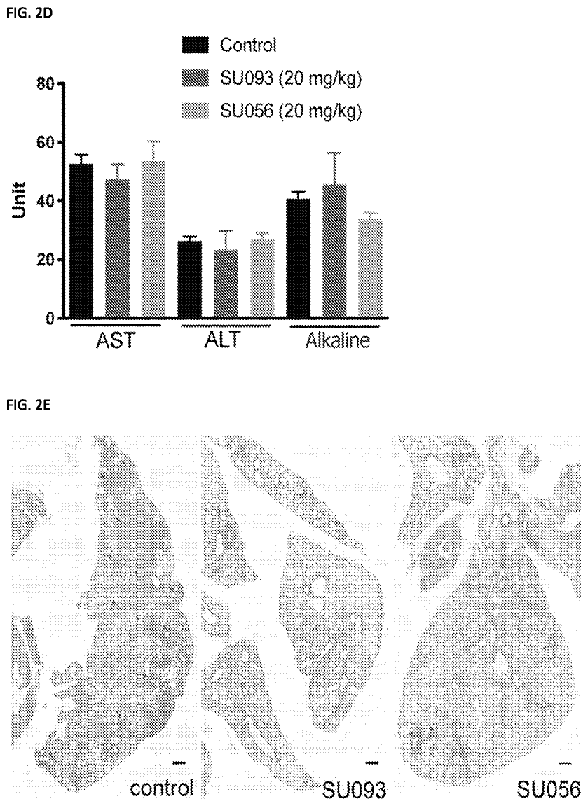

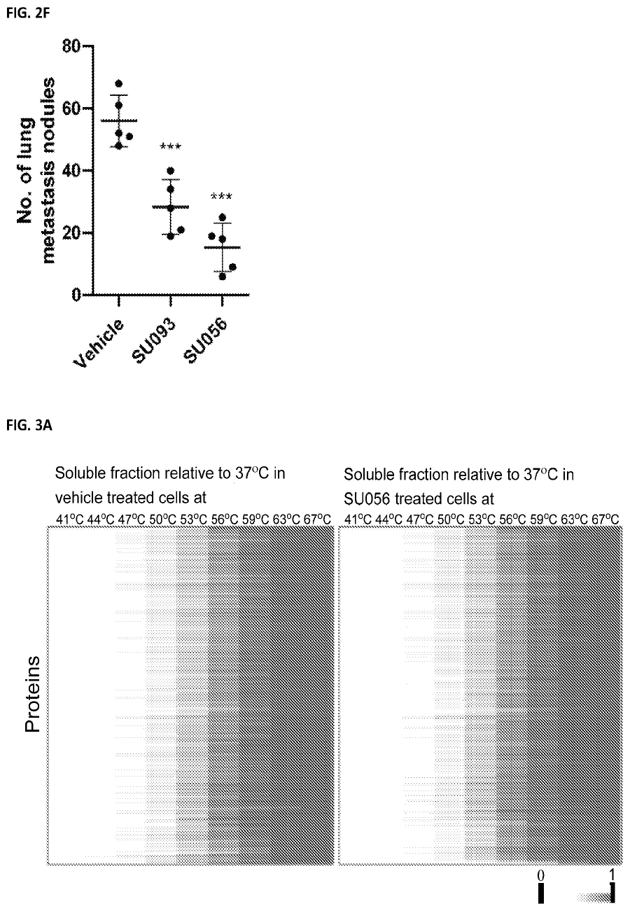

These promising results led us to test SU093 and SU056 in vivo. C57BL/6 mice were implanted with 2×10⁶ firefly luciferase (luc+)-expressing ID-8 syngeneic ovarian cancer cells subcutaneously. Once the tumors reached 100 mm³ in size, we began daily intraperitoneal treatment with SU093 and SU056 at 20 mg/kg (vehicle control 30% PEG-300 in saline). Tumor growth was monitored via bioluminescence imaging (IVIS). SU093 and SU056 treatment significantly delay the tumor progression than vehicle control through the treatment period (FIGS. 2A and 2B). At 42 days, a final assessment of the tumor weight showed that SU056 treatment resulted in a 2-fold reduction in the tumor weight, whereas SU093 shrunk tumors by 1.5-fold (FIG. 2C). This was accompanied by no significant impact on blood chemistry indicators such as aspartate aminotransferase (AST), alanine aminotransferase (ALT), or alkaline phosphatase (FIG. 2D), showing that daily SU056 and SU093 are well-tolerated treatment regimens.

Due to prior observation of a significant reduction in the migratory ability of OC cells treated with the two drugs (FIG. 1H), we also measured the effect of drug treatment on ovarian cancer metastases. At the end of above experiment, lungs were fixed and metastatic foci were counted under the dissecting microscope followed by H&E staining. Measurement of the metastasis in SU056-treated mice demonstrated a 3-fold reduction in lung metastases (FIGS. 2E and 2F), as assessed via histological scoring. Based on these data and superior performance in all assays, we selected SU056 for further study.

Target Identification: SU056 Interacts with Proteins Associated with Oncogenesis

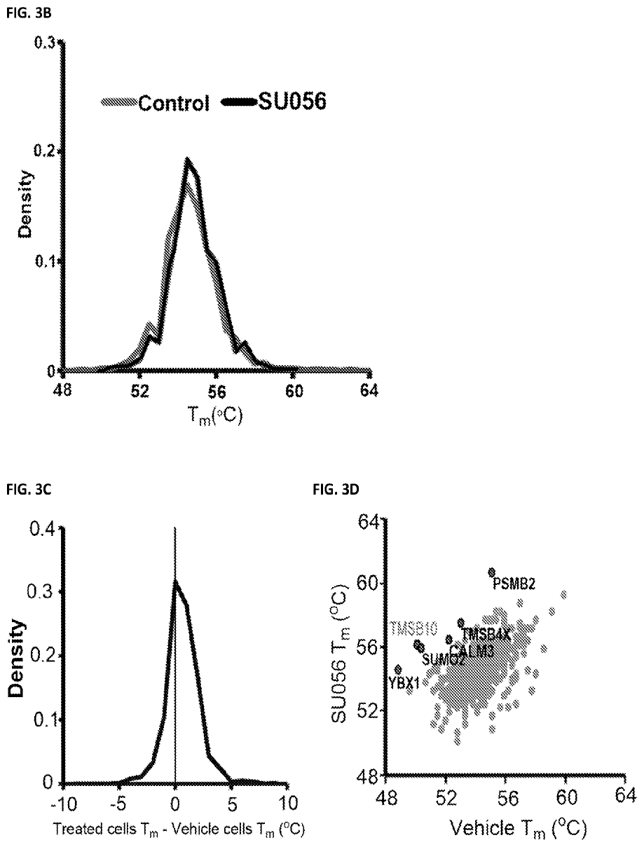

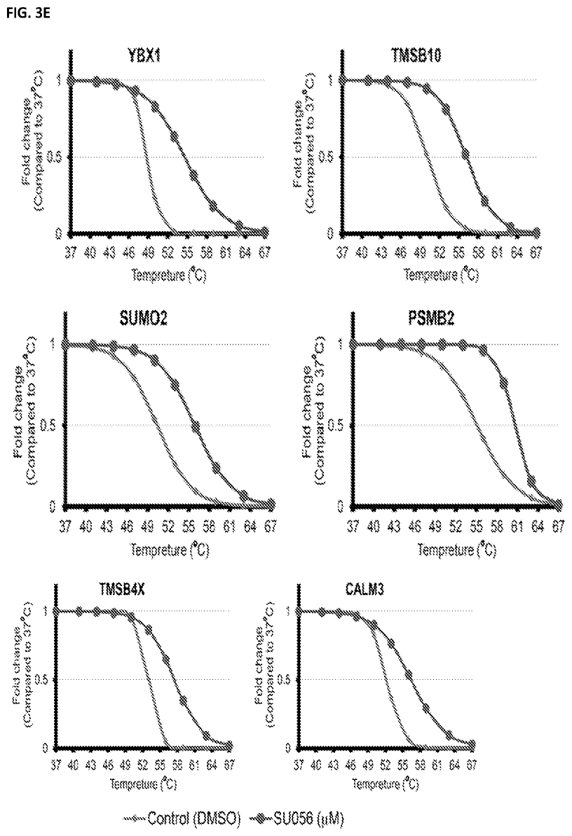

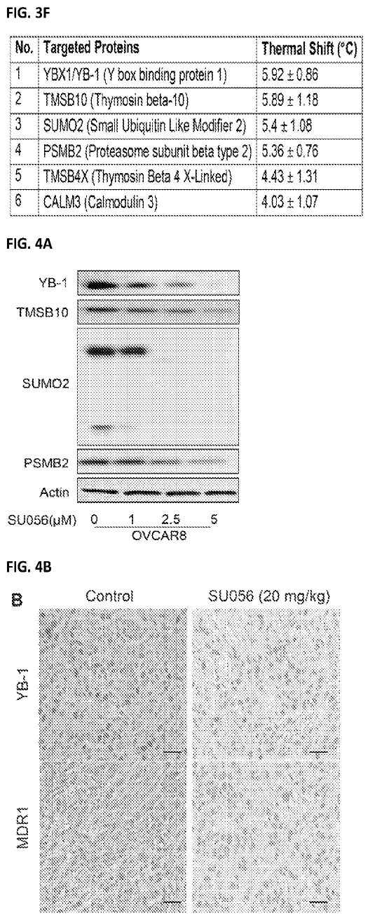

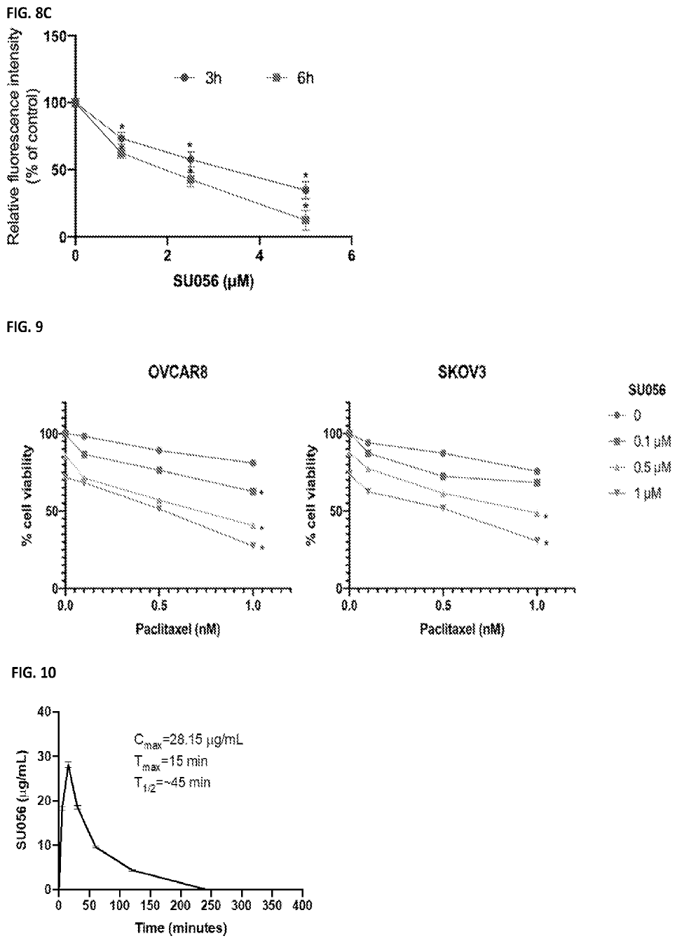

The cellular thermal shift assay (CETSA) is based on the principle of protein-ligand interactions causing a shift in protein thermal stability (Savitski et al., 2014). When a protein interacts with ligand, its thermal stability increases, and this principle allows identification of which protein targets that are engaged by small molecules or drug candidates. We used this assay to identify protein targets of SU056. OVCAR8 cells were treated with SU056 (2.5 μM) for 1.5 h, and protein lysates were analyzed for a thermal shift. The heat maps represent the thermal stability of 804 soluble proteins in the presence and absence of SU056 compare with 37° C. (FIG. 3A). 77% of proteins were identified in both vehicle control and SU056 treated cells and 97% of these proteins passed criteria to be adjusted to the melting curves. The compression of thermal stability between DMSO- and SU056-treated proteomes suggests that SU056 treatment increases the overall stability of cellular proteome (FIGS. 3A, 3B, and 3C). Out of 804 soluble proteins, SU056 treatment significantly increased the thermal stability of six proteins whose p-value<0.01 and RSQ>0.7 (FIG. 3D). These six proteins are YBX1 (YB-1), TMSB10, SUMO2, PSMB2, TMSB4X, and CALM3 (FIG. 3D). Melting curves of these proteins with and without SU056 suggest that SU056 increases thermal stability by 5.92±0.86, 5.89±1.18, 5.4±1.08, 5.36±0.76, 4.43±1.31, and 4.03±1.07° C. of YB-1, TMSB10, SUMO2, PSMB2, TMSB4X, and CALM3, respectively (FIGS. 3E and 3F). This result was further validated using western blotting (WB) (FIG. 8A). All six of these proteins have previously reported to contribute to carcinogenesis.

### SU056 Treatment Inhibit the YB-1

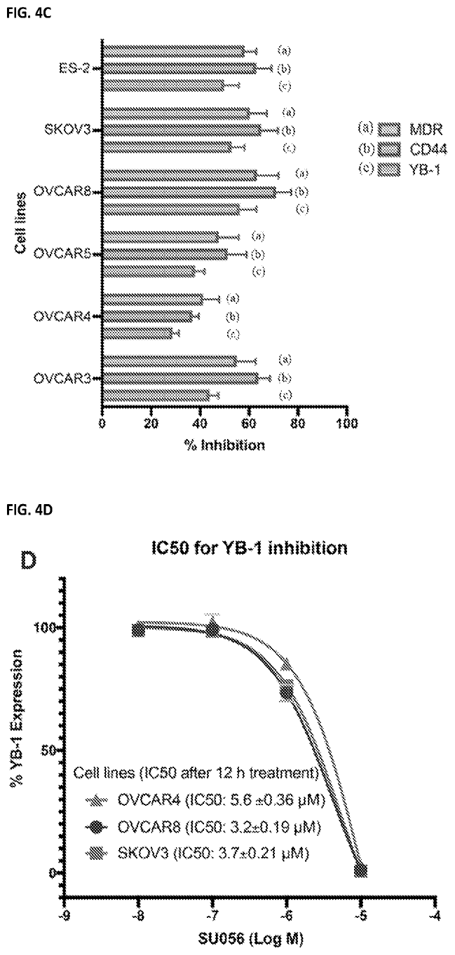

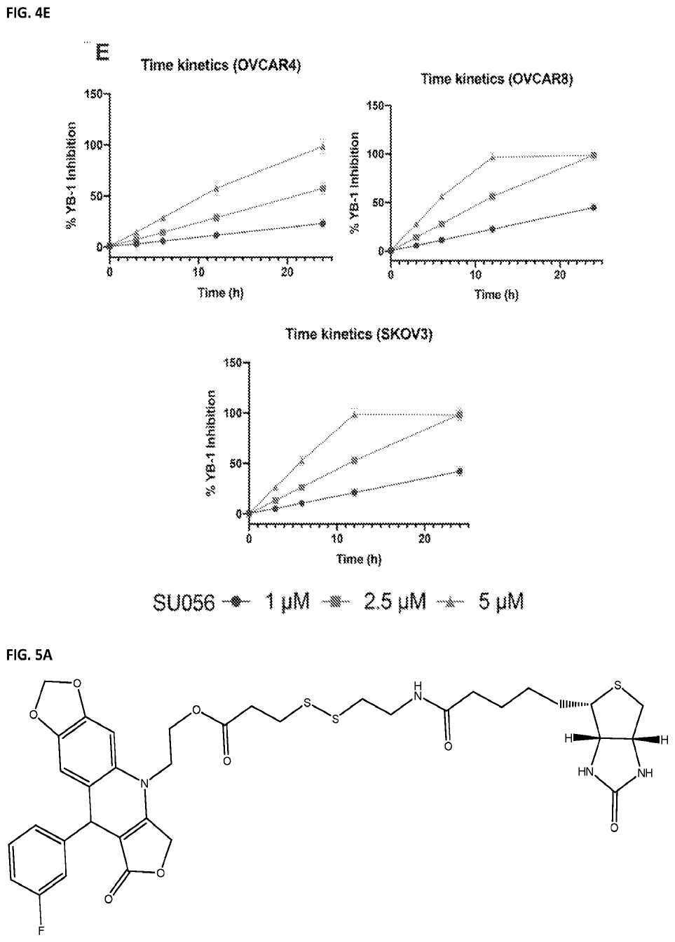

Out of six targets identified, we selected proteins whose thermal shift is above 5° in the presence of SU056 for further study of protein expression after treatment. Western blot analysis of OVCAR8 cell treated with DMSO and SU056 (1, 2.5 and 5 μM) for 12 h suggests that SU056 treatment inhibits the YB-1, TMSB10, SUMO2 and PMSB2 proteins in a dose-dependent manner (FIG. 4A). SU056 strongly inhibits YB-1 expression and, moreover considering the role of YB-1 in interacting with many oncogenic proteins treatment resistance factors led us to select YB-1 for further validation. To study the in vivo effects of SU056 treatment, we analyzed ID8 tumor xenograft samples via immunohistochemistry (IHC). IHC for YB-1 and MDR1 expression in vehicle and SU056 (20 mg/kg) treated tumors suggests that SU056 strongly inhibits YB-1 expression followed by downregulation of downstream MDR1 (FIG. 4B). To identify the broader effects of SU056 on YB-1 and its downstream protein/activity in different ovarian cancer cell lines, we selected six different cell lines and treated them with SU056 (2.5 μM) for 12 h. Cells were analyzed for YB-1 and CD44 expression and Multidrug resistance 1 (MDR1, ABC pump mediated efflux). Results suggest that this treatment inhibits YB-1 expression by 28-56% and is accompanied by a 36-70% decrease in CD44 expression and 41-63% decrease in MDR expression (FIG. 4C). This result suggests that SU056 broadly inhibits YB-1 across different OC cell lines irrespective of their genetic background and stage of cancer. To calculate the 50% YB-1 inhibitory concentration (IC₅₀) of SU056, we treated OVCAR4, OVCAR8, and SKOV3 cell lines with SU056 (0.01, 0.1, 1, 10 μM) for 12 h and YB-1 expression was analyzed using YB-1 ELISA. The IC₅₀ values for OVCAR4, OVCAR8, and SKOV3 were 5.6±0.36 μM, 3.2±0.19 μM, and for 3.7±0.21 μM, respectively (FIG. 4D). Finally, we tested the time kinetics and dose-dependent effects of SU056 on YB-1 expression. OVCAR4, OVCAR8, and SKOV3 cells were treated with SU056 (1, 2.5, and 5 μM) for 3, 6, 12, and 24 h and YB-1 expression was measured using YB-1 ELISA. Results suggest that SU056 treatment inhibits YB-1 in a time- and dose-dependent manner (FIG. 4E). 5 μM treatment for 24 h inhibited YB-1 completely (100%) in all three OC cells (FIG. 4E). This result was further conformed using OVCAR8 cells stably expressing mCherry tagged YB-1. Confocal imaging and fluorescence intensity measurement of YB-1 OVCAR8 cells also suggest that SU056 treatment inhibit the YB-1 expression time and dose dependent manner (FIGS. 8B and 8C). Overall, results of YB-1 expression and its downstream factors (FIG. 4) suggest that SU056 is a strong inhibitor of YB-1 protein and its activity.

### SU056 Biophysically Binds to YB-1 and its Cellular Activity is YB-1 Dependent

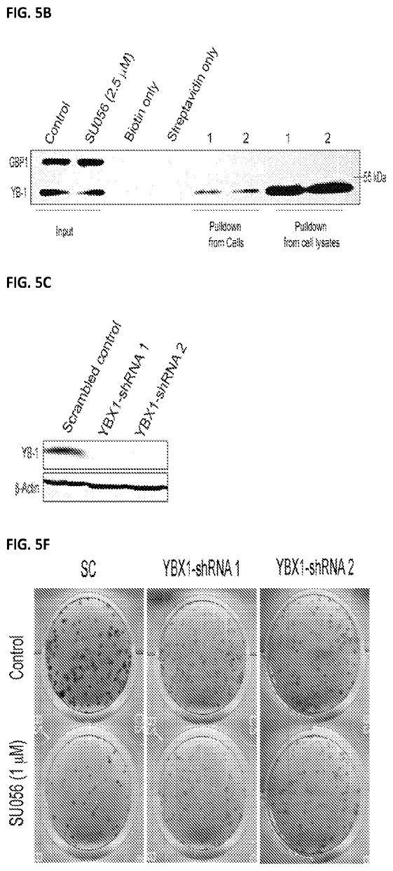

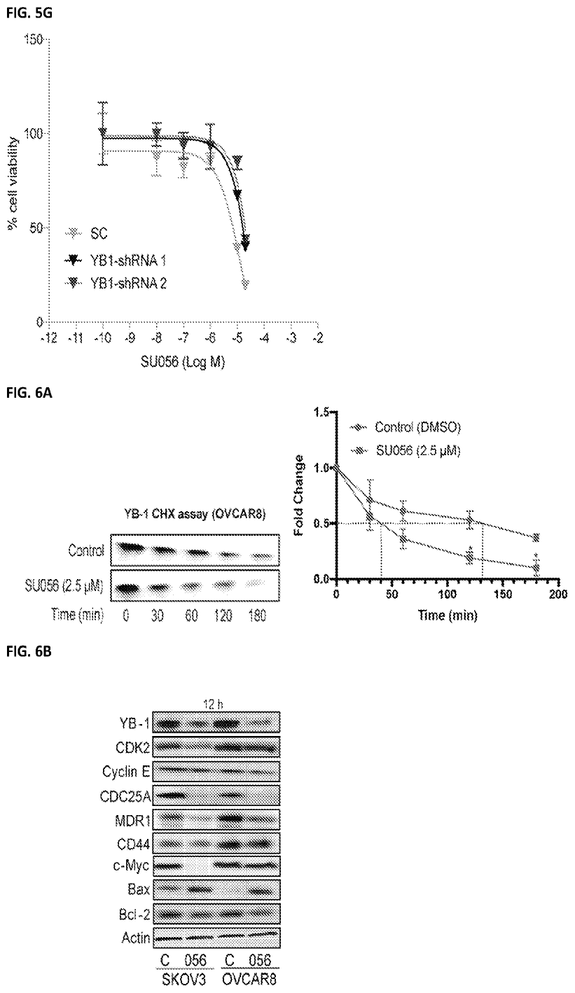

Previously, our group reported that SU093 interacts with GBP1 and inhibits the GBP1:PIM1 interaction (Andreoli et al., 2014). SU056 is the second generation small molecule derivative of SU093 and, to confirm whether it has the same molecular target as SU093, we performed a pulldown assay using biotinylated SU056 (FIG. 5A). Pulldown from cells treated with Biotinylated-SU056 and from protein lysate indicates that SU056 physically interacts with YB-1 but not with GBP1 (FIG. 5B). This result suggests that, although SU093 and SU056 have structural similarity, their targets are quite different. In further support this, we performed a Surface Plasma Resonance (SPR) screen to measure the orthogonal biophysical binding between YB-1 and SU093 or SU056. His-tagged YB-1 protein was immobilized on a GE NTA chip and different concentrations (1-100 μM) of SU093 and SU056 were tested for binding (FIG. 5CD). SPR results also support that SU056:YB-1 has a strong biophysical interaction (FIG. 5D) compared to SU093 (FIG. 5C). To check the dependency of SU056 activity on YB-1 expression, we also tested OVCAR8 cells transfected with YB-1 knockdown using transduction of lentiviral shRNA vectors. Two different shRNA sequences (YBX1-shRNA1 and YBX1-shRNA2) were used and both yielded 80-90% inhibition in the expression of YB-1 compared to scrambled control (SC)-transduced cells (FIG. 5E). A clonogenic assay was performed using these three cell lines with and without SU056 (1 μM). Results suggest that YB-1 knockdown cells are more resistant to SU056 treatment compared to SC cells (FIG. 5F). Furthermore, we also performed a cell viability assay using knockdown cells to calculate IC₅₀ values. The IC₅₀ of SU056 for SC cells was 3.54 μM (±0.21) and for YBX1-shRNA1 and YBX1-shRNA2 was 15.84 (±0.13) and 19.15 (±0.34), respectively (FIG. 5G). YB-1 knockdown increased the IC₅₀ value ˜5 fold compared to SC. These results show that the effect of SU056 is dependent on cellular expression of YB-1.

### SU056 Modulates the YB-1 Associated Proteins and Pathways

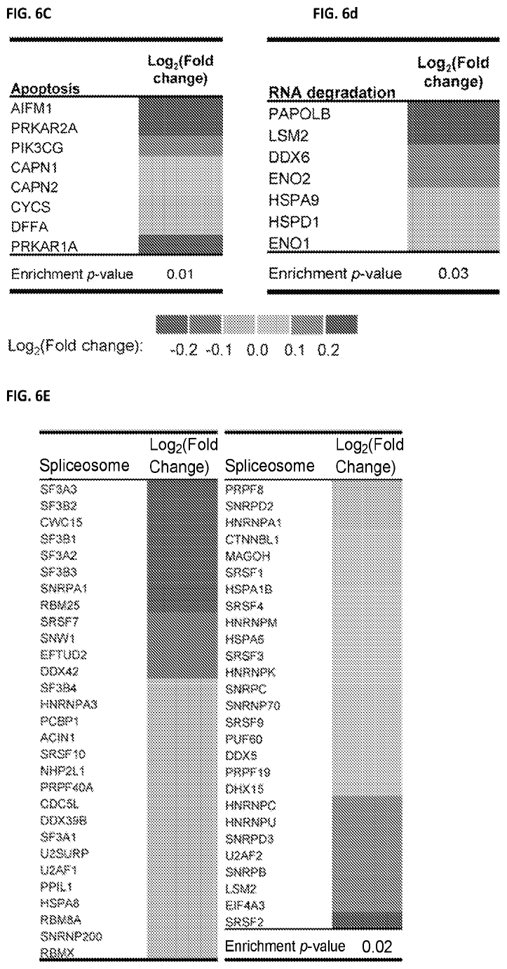

CETSA results show that SU056 physically interacts with YB-1 and inhibits it and its activity. To study the effect of SU056 on YB-1 protein stability, we used the Cycloheximide chase (CHX) assay. Cycloheximide is a protein translation inhibitor and is used as a molecular biology tool to determine the half-life of proteins. OVCAR8 cells were treated with CHX and DMSO or SU056 (2.5 μM), and protein lysates were collected at different time points (0, 30, 60, 120, 180 min) followed by YB-1 immunoblotting. Results suggest that SU056 treatment lead the proteasomal degradation and reduces the half-life of YB-1 to from ˜130 minutes to ˜40 minutes (FIG. 6A). YB-1 is associated with the transcription and translation of many oncogenic proteins including CD44, ABCB1/MDR1, c-Myc, and Bcl-2. Immunoblotting of whole-cell lysates from DMSO- or SU056- (2.5 μM, 12 h) treated SKOV3 and OVCAR8 cells indicate that SU056 treatment inhibits YB-1 protein (FIG. 6B). This was followed by a decrease in the expression of CDK2, CDC25A, MDR1, CD44, c-Myc, and Bcl-2 and an increase in the expression of pro-apoptotic protein Bax (FIG. 6B). To evaluate proteome changes lead by SU056, we undertook proteome profiling of DMSO- and SU056- (2.5 μM, 12 h) treated OVCAR8 cells. GSEA KEGG analysis of the resulting proteomics data suggest that SU056 treatment significantly induces apoptosis (ES=0.792, p-value-0.01), RNA degradation (ES=0.783, p-value-0.03), Alanine, aspartate, and glutamate metabolism (p-value-0.02), Arginine and proline metabolism (p-value-0.04), FcεRI signaling pathway (p-value-0.01), T-cell receptor signaling pathway (p-value-0.02), Natural killer cell-mediated cytotoxicity (p-value-0.03), Epithelial cell signaling in Helicobacter pylori infection (p-value-0.04), and FcγR mediated phagocytosis pathways (p-value-0.05) (FIG. 6C-D, Table 1). On the other hand, SU056 treatment also inhibits the spliceosome pathway (ES=−0.413, p-value-0.02) (FIG. 6CE, Table 1). YB-1 protects and stabilizes the mRNA via 5′ end capping (Evdokimova et al., 2001) and enhances exon splicing (ex. Alternative splicing of CD44) (Stickeler et al., 2001). YB-1 inhibition via SU056 treatment inhibits the Spliceosome pathway and induces RNA degradation and apoptosis. These results suggest that SU056 primarily targets YB-1, in turn inhibits various YB-1 associated proteins and pathways involved in cancer progression and TR.

### YB-1 Inhibition via SU056 Treatment Sensitizes OC to Paclitaxel

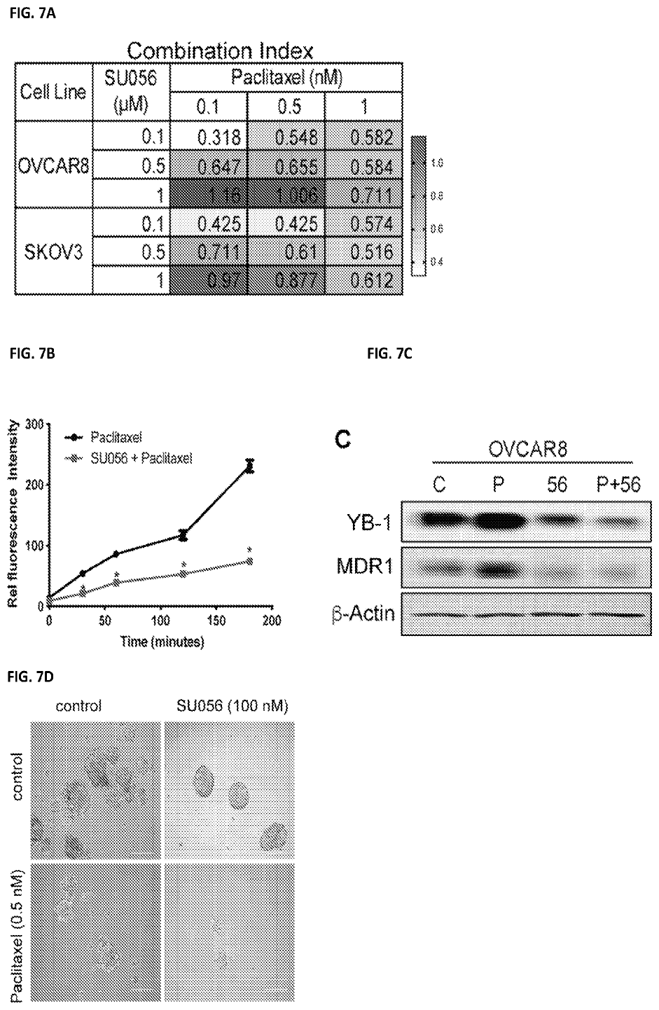

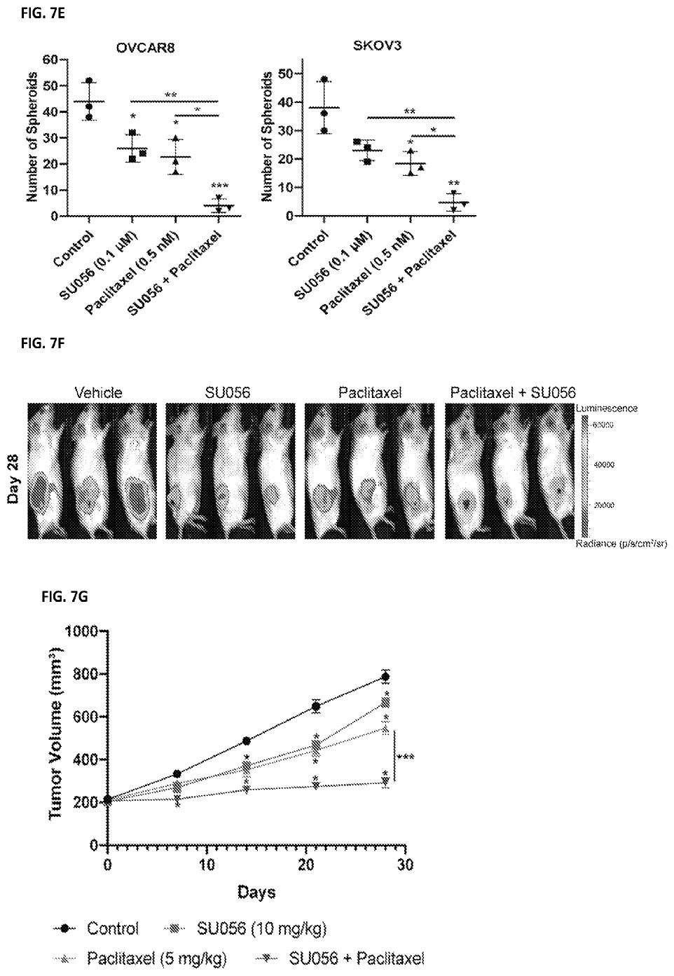

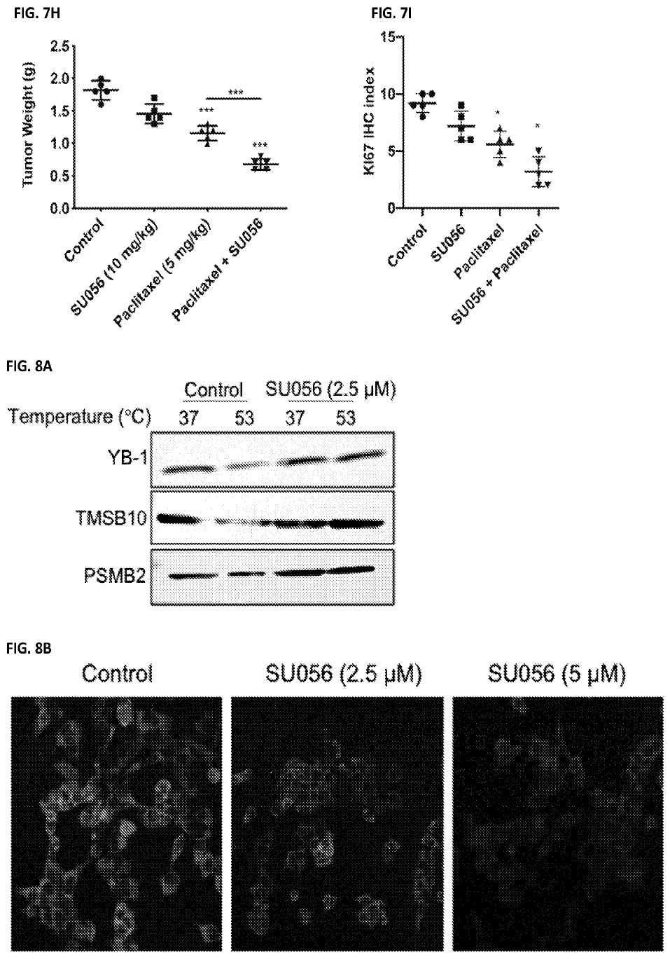

YB-1 is involved in the emergence of cisplatin and taxane drug resistance (Kang et al., 2013, Mo et al., 2016). Therefore, we tested whether SU056 synergizes with chemotherapies such as paclitaxel to modulate YB-1. To measure this, we performed a dose dependent combination study of SU056 and paclitaxel. Using the MTT assay, we found that SU056 treatment significantly potentiates the cytotoxic effects of paclitaxel at 0.1, 0.5, and 1 nM doses (Figure S2, 7A). Treatments with 0.5 or 1.0 μM SU056 significantly reduced cell viability in both OVCAR8 and SKOV-3 OC cell lines. We calculated the combination index (CI) using the Chou-Talalay method and found a CI value of <1 for SU056 and paclitaxel combination. CI<1 suggests significant synergy of SU056 with the growth inhibitory effect of paclitaxel (FIG. 7A). ABCB1/MDR1 drug efflux pumps are also known to play an important downstream role with YB-1 in increased taxol efflux and subsequent TR in cancer. To evaluate whether SU056 affects drug efflux, we used Alexa Fluor-488-tagged paclitaxel to measure taxol efflux in vitro. Results suggested that SU056 co-treatment significantly inhibits the efflux of paclitaxel in comparison to only paclitaxel-treated cells (FIG. 7B). Efflux is primarily driven by the ATP-binding cassette transporters (ABC pumps) present on the cell surface (Genovese et al., 2017). We examined the expression of MDR1 and YB-1 in OVCAR8 cells treated with vehicle, SU056, and paclitaxel combinations via Western blot. Immunoblot results show that paclitaxel treatment alone is sufficient to upregulate the expression of YB-1 and MDR1, which is then reversed by the combined treatment of SU056 and paclitaxel (FIG. 7C). These results strongly suggest that SU056 synergizes with the efficacy of paclitaxel.

3D spheroids are an alternative tumor model that closely mimic the TR phenotype via increased drug efflux. We cultured OVCAR8 and SKOV3 cells in ultralow attachment plates with growth factor-defined media in the presence or absence of paclitaxel and SU056. Results show that a combination of SU056 and paclitaxel significantly inhibits the formation by OC cell spheroids (FIGS. 7D and 7E). Combination-treated cells form 78% (OVACR8) and 83% (SKOV3) fewer spheroids than the vehicle-treated cells (FIG. 7E).

In a pharmacokinetics study in vivo, injection of 20 mg/kg SU056 led to a maximum serum concentration of 28.19 μg/mL with a T_{1/2} of ˜45 min. We then tested the synergism of paclitaxel and SU056 co-treatment in vivo in NOD-SCID mice implanted with OVCAR8 OC tumors as described above. Mice were given daily treatment of SU056 (10 mg/kg) and weekly treatment of paclitaxel (5 mg/kg) were administered intraperitoneally. Both paclitaxel and SU056 treatment independently showed significant reduction in OC tumor growth. However, the combination of paclitaxel and SU056 demonstrated a much greater reduction in OC tumor growth, effectively stabilizing disease progression over the treatment period (FIGS. 7F and 7G). The drug combination showed sustained inhibitory effects on tumor growth until the experiment terminated (FIG. 7H). We also measured the proliferative index within the excised tumor using Ki-67 staining (FIG. 7I), which showed that both SU056 and paclitaxel exerted independent anti-proliferative effects that synergized to produce a greater therapeutic effect when combined. This likely resulted from decreased paclitaxel efflux from OVCAR8 cells in the presence of SU056.

### Discussion

Ovarian cancer (OC) is the fifth most common cancer in women with an overall 5-year survival rate of just 47.6% in the United States of America (USA). Surgical resection and chemotherapy are the primary treatments for OC but are limited by surgical difficulties due to the abdominal spread of metastasizing OC and a high 2-year relapse rate of 80-90% after taxane or platinum chemotherapy treatment (Jayson et al., 2014, Agarwal and Kaye, 2003). While paclitaxel offers some effect, relapsed disease is frequently TR, in which tumor cells bypass or overcome the molecular mechanisms of cytotoxicity despite ongoing treatment (Singh and Settleman, 2010, Blagosklonny and Fojo, 1999, Horwitz et al., 1986). An investigation into the mechanisms of TR in taxane-resistant OC by the Sood group revealed significantly upregulated YB-1 in treated OC patients where patients with high YB-1 expression had significantly shorter overall survival (Kang et al., 2013). Here, we report fluorine-based derivatives of podophyllotoxins as new potent and highly effective YB-1 inhibitors capable of restraining disease progression and synergizing with chemotherapy.

Overexpression of βIII-tubulin is a prominent signature of paclitaxel-TR and the GBP1:PIM1 interaction may help activate its function (De Donato et al., 2012, Mariani et al., 2011). Our group previously reported a small molecule podophyllotoxins (SU093) as an inhibitor of the GBP1:PIM1 interaction capable of overcoming taxane resistance in vitro (Andreoli et al., 2014). To improve the potency of SU093, we constructed a second-generation compound library through a structure-guided and bioisostere replacement strategy. Screening of this library found that a fluorine-based derivative, SU056, has markedly improved potency and safety with a different mechanism of action. We screened SU056 for its cytotoxic effects and found improved efficacy compared to SU093. SU093 and SU056 both caused G1 cell cycle phase arrest, increased apoptotic cell death, and inhibited cell migration. Both compounds also inhibited tumor progression and metastasis in the ID8 xenograft model. During treatment, neither compound caused any liver toxicity. In each assay, SU056 proved more potent than SU093 or other AzP derivatives, and therefore selected for further study.

We used the Cellular Thermal Shift Assay (CETSA) to identify targets of SU056 and found that this compound interacts with YB-1, TMSB10, SUMO2, PSMB2, TMSB4X, and CALM3. Target identification suggested that SU056 inhibits proteins that have an oncogenic role in ovarian and/or other cancers. SU056 treatment was found to decrease the expression of YB-1 in a dose and time-dependent manner in different OC cell lines. Our experiments validated that SU056 treatment strongly interact with YB-1 and inhibits and its associated downstream proteins and pathways. SU056 arrests the OC cells in the G1 phase and also inhibits the key drivers of G1/S phase (CDK2 and CDC25A). On the other hand, YB-1 plays a role in the phosphorylation and activation of CDC25A to drive G1/S phase progression (Zhao et al., 2016), and the knockdown of YB-1 leads the G0/G1 phase arrest (Harada et al., 2014). CD44, c-Myc, and MDR1 are the most prominent oncogenic downstream proteins regulated by YB-1, and SU056 treatment significantly inhibits their expression. YB-1 is a mRNA binding protein involved in nucleoprotein filament formation in cytoplasm (Kretov et al., 2019). Together with YB-2 and YB-3, YB-1 binds with the cold-shock domain of single-stranded RNA/DNA (Graumann and Marahiel, 1998). It is involved in shuttling nucleic acids in both cytoplasm and nucleus (Matsumoto and Wolffe, 1998). In the cytoplasm, it regulates RNA stability, translation activity, and alternative splicing (Chansky et al., 2001). In the nucleus, it binds to the specific promoter sequences to regulate the transcription of oncogenic proteins, including MDR1 (Bargou et al., 1997). SU056-mediated YB-1 inhibition significantly upregulates the RNA degradation pathway, while inhibiting the spliceosome pathway; a result which concords with the literatures about YB-1 function. Collectively, SU056:YB-1 interaction inhibits the YB-1 activity in different OC cell line via proteasomal degradation and inhibits various downstream factors involved in tumor progression and TR. These results confirm YB-1 inhibition-specific activity of SU056 as a new drug candidate.

Furthermore, we also investigated the effect of SU056 in combination with paclitaxel, and we found that lower doses of 10 mg/kg of SU056 potentiate the cytotoxic effect of paclitaxel treatment. This effect may be because co-treatment with SU056 lowers the efflux rate of paclitaxel in OC cells. Drug efflux by cancer cells is one of the important mechanisms for the development of TR (Li and Nikaido, 2009, Gottesman and Pastan, 2015). Efflux is primarily driven by ATP binding cassette (ABC) transporters present on cell surfaces. This superfamily includes ABCB1, also known as multidrug resistance 1 (MDR1), P-glycoprotein, ABCC1 (MRP1), and ABCG2 (BCRP/MXR) (Fletcher et al., 2010). Paclitaxel is a substrate for ABCB1, and paclitaxel treatment upregulates its expression in various cancer types (Gottesman and Pastan, 1993). ABC transporters are a hallmark of both cancer progression, and TR and has been viewed as a potential therapeutic target. ABCB1 chemical inhibitors have been developed, but first, second, and third-generation compounds failed in clinical trials thus far. First-generation compounds had toxicity issues, whereas second-generation compounds such as Valspodar, showed no treatment benefit in combination with paclitaxel or carboplatin in ovarian or peritoneal cancer patients (Lhomme et al., 2008). The third-generation inhibitor Zosuquidar also failed to show any benefits (Ruff et al., 2009), leaving the inhibition of MDR1 and TR phenotype an unmet clinical goal. In testing the effects of paclitaxel, SU056, and their combination on the expression of MDR1 and YB-1, we found that paclitaxel treatment upregulates the expression of both YB-1 and MDR1, whereas SU056 alone or in combination with paclitaxel significantly decreases MDR1 and YB-1. This is supported by paclitaxel efflux data showing that SU056 treatment inhibits MDR1 and leads to higher intracellular concentrations and activity of paclitaxel.

Tumor spheroids, a common 3D cell culture model, have been used to examine stemness, efflux, and MDR1 expression changes after treatment (Wartenberg et al., 2005, Wartenberg et al., 1998, Chen et al., 2017). In our study, treating OC cells with a combination of paclitaxel and SU056 significantly inhibited their spheroid formation capability, which indicates decreased tumorigenic potential. Previous transcriptome analysis of taxane-resistant ovarian cancer cell lines has revealed elevated levels of both YB-1 and MDR1 (Sun et al., 2015, Kuwano et al., 2004, Shiota et al., 2014, Wu et al., 2007). YB-1 is associated with transcriptional regulation of ABC transporters and epithelial-to-mesenchymal transition-associated proteins implicated in disease progression and TR (Lim et al., 2018, Wu et al., 2014, Evdokimova et al., 2009). SU056 co-treatment with paclitaxel was sufficient to halt tumor progression in the OVCAR8 xenograft model. This further suggests that SU056 and the analogs thereof described herein provide a promising strategy for OC, rescuing treatment efficacy, and increasing patient survival.

In summary, bioisostere replacement was a highly effective chemical strategy to optimize SU093 for improved efficacy and reduced toxicity, resulting in the development of SU056 and the corresponding analogs discussed herein. These AzP derivatives are the first inhibitors reported for YB-1, ultimately decreasing cell proliferation and migration, while sensitizing OC cells to the cytotoxic effects of paclitaxel in vitro and in vivo. Additional work to characterize the impact of SU056 treatment on different animal models, other cancer types, and the immune microenvironment will provide further insight into the potential of SU056 and its analogs to combat treatment resistance in the clinic.

### Materials and Methods

Compound synthesis: AzP derivative SU093 was synthesized and characterized as reported previously (Andreoli et al., 2014). SU056 and biotinylated SU056 were synthesized following protocol described below.

Synthesis of SU056

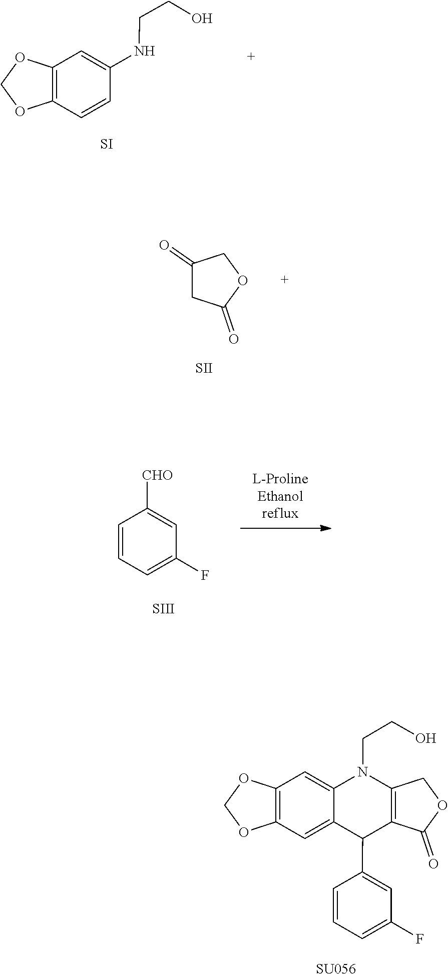

A solution of aryl amino alcohol SI (Kumar et al., 2010) (1 mmol), 3-fluorobenzaldeyde (SIII) (1.2 mmol), L-proline (0.1 mmol, 10 mol %), and tetronic acid (SII) (1.2 mmol) was prepared in anhydrous ethanol (4 mL) and the reaction mixture was refluxed for 3-4 h. Upon consumption of amino alcohol component and appearance of a fluorescent spot via TLC (9:1 of 50% EtOAc/Hex:MeCN), a slurry of silica gel was prepared and purified by flash chromatography to give SU056 as a solid (0.203Gm 50%). ¹H NMR (400 MHz, DMSO-d₆) δ 7.27 (td, J=7.9, 6.1 Hz, 2H), 7.10-6.98 (m, 4H), 7.01-6.91 (m, 1H), 6.96 (s, 2H), 6.67-6.62 (m, 2H), 5.97 (d, J=1.1 Hz, 2H), 5.91 (d, J=1.1 Hz, 2H), 5.15-5.00 (m, 4H), 5.04-4.96 (m, 2H), 4.94 (s, 2H), 3.81 (d, J=10.5 Hz, 1H), 3.71-3.58 (m, 3H). ¹³C NMR (101 MHz, DMSO-d₆) δ 172.59, 161.11, 150.24, 147.52, 143.76, 131.50, 130.66, 130.58, 124.02, 118.94, 114.77, 114.56, 113.69, 110.48, 101.88, 96.83, 94.82, 66.31, 58.38, 48.64. MS-ESI m/z calculated for C₂₀H₁₆FNO₅ [M+H]⁺: 370.1, found 370.1.

Synthesis of Biotinylated SU056

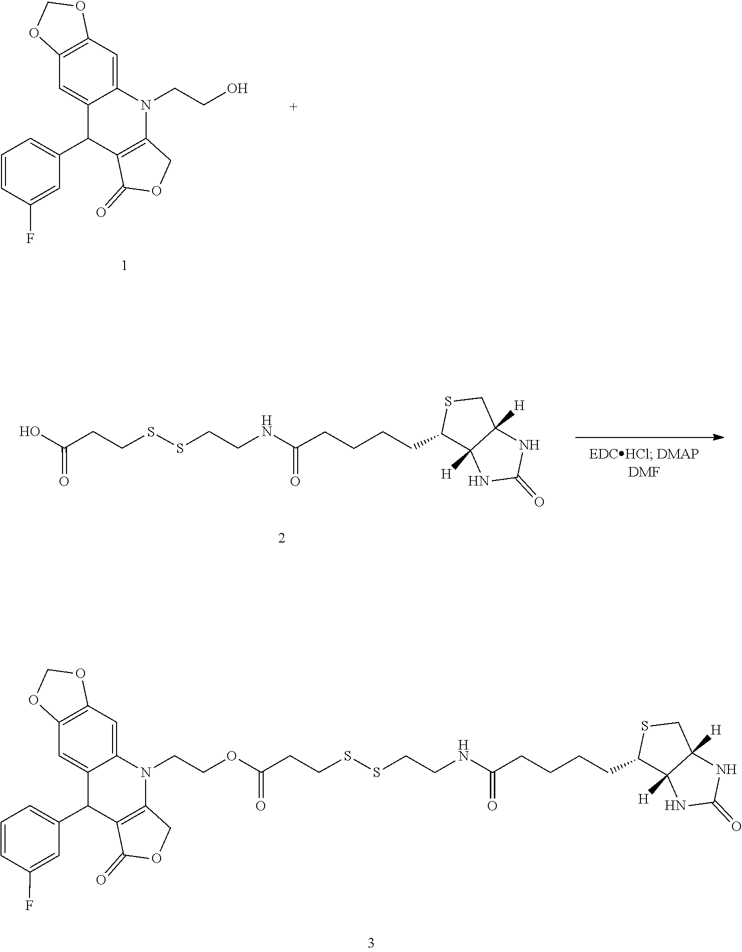 [SMILES: O=C1OCC2=C1C(c1cccc(F)c1)c1cc3c(cc1N2CCO)OCO3 ; O=C(O)CCSSCCNC(=O)CCCC[C@@H]1SC[C@@H]2NC(=O)N[C@@H]21 ; O=C(CCCC[C@@H]1SC[C@@H]2NC(=O)N[C@@H]21)NCCSSCCC(=O)OCCN1C2=C(C(=O)OC2)C(c2cccc(F)c2)c2cc3c(cc21)OCO3]

2-(9-(3-fluorophenyl)-8-oxo-6,9-dihydro-[1,3]dioxolo[4,5-g]furo[3,4-b]quinolin-5(8H)-yl)ethyl 3-((2-(5-((3aS,4S,6aR)-2-oxohexahydro-1H-thieno[3,4-d]imidazol-4-yl)pentanamido)ethyl)disulfaneyl) propanoate: Under a nitrogen atmosphere, a solution of 9-(3-fluorophenyl)-5-(2-hydroxyethyl)-6,9-dihydro-[1,3]dioxolo[4,5-g]furo[3,4-b]quinolin-8(5H)-one (40.0 mg, 0.108 mmol) in dry DMF (10.0 mL) was treated with 3-[2-N-(Biotinyl)aminoethyldithio]propanoic acid (44.0 mg, 0.108 mmol), EDC·HCl (31.1 mg, 0.162 mmol) and DMAP (19.8 mg, 0.162 mmol). The reaction mixture was stirred at rt for 18 h then diluted with water (˜10 mL). The mixture was then extracted with ethyl acetate (2×25 mL). Combined organic phase was washed with water followed by brine solution. The organic phase was dried over Na2SO4, filtered, and evaporated to dryness. The crude was purified with CombiFlash chromatography on silica gel using 0-10% of methanol in dichloromethane as an eluent. Combined pure fractions was evaporated to dryness to afford the desired product as an off-white solid (15.0 mg, 19%). 1H NMR (400 MHz, Chloroform-d) δ 7.28-7.21 (m, 1H), 7.14-6.98 (m, 1H), 6.97-6.78 (m, 2H), 6.65-6.50 (m, 2H), 6.42-6.21 (m, 1H), 5.98 (dt, J=10.5, 1.3 Hz, 2H), 5.38-5.23 (m, 1H), 5.15-4.88 (m, 3H), 4.71-4.30 (m, 3H), 4.14-3.91 (m, 1H), 3.90-3.79 (m, 1H), 3.75 (q, J=7.0 Hz, 1H), 3.61-3.32 (m, 2H), 3.19 (td, J=7.4, 4.6 Hz, 1H), 2.95 (ddd, J=12.8, 5.0, 1.3 Hz, 1H), 2.85 (dd, J=8.3, 6.2 Hz, 2H), 2.80-2.65 (m, 2H), 2.34-2.13 (m, 4H), 1.70 (dq, J=14.7, 8.1, 7.4 Hz, 3H), 1.49 (q, J=7.8 Hz, 3H), 1.36-1.13 (m, 2H). LC-MS (ESI-QQQ): m/z 759.2 ([C35H39FN4O8S3+H]+ calcd. 759.2). Purity>98% (rt 4.64 min).

General Synthesis: Compounds described herein may also be prepared by methods known in the art, including those described in the article Synthesis of Novel Functionalized 4-Aza-2,3-didehydropodophyllotoxin Derivatives with Potential Antitumor Activity, Kumar et al., J. Heterocyclic Chem., 47(6), 1275-1282, November 2010.

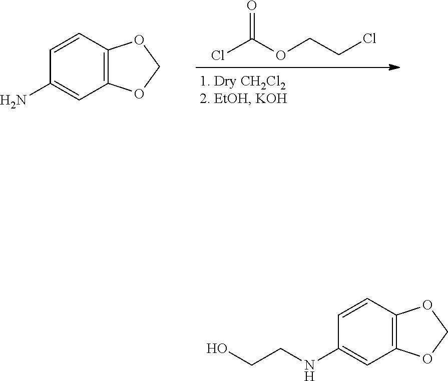 [SMILES: Nc1ccc2c(c1)OCO2 ; O=C(Cl)OCCCl ; OCCNc1ccc2c(c1)OCO2]

2-(benzo[d][1,3]dioxol-5-ylamino)ethan-1-ol can be prepared by slowly adding 2-chloroethyl carbonochloridate to benzo[d][1,3]dioxol-5-amine in dry dichloromethane and pyridine. The mixture is stirred at room temperature for 2.5 hours, washed with water, and dried over anhydrous magnesium sulfate and concentrated under vacuum prior to additional washing, concentration, and drying.

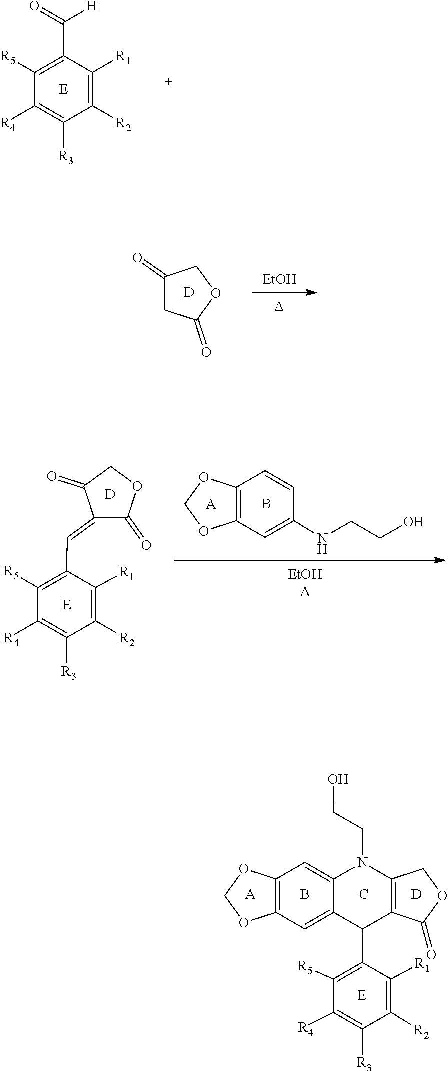

A benzaldehyde compound of desired substitution can be reacted with tetronic acid in ethanol and refluxed for 30 to 90 minutes to form the comparably substituted (Z)-3-benzylidenefuran-2,4(3H,5H)-dione, which then may be reacted with 2-(benzo[d][1,3]dioxol-5-ylamino)ethan-1-ol to obtain the final substituted 5-(2-hydroxyethyl)-9-phenyl-6,9-dihydro-[1,3]dioxolo[4,5-g]furo[3,4-b]quinolin-8(5H)-one of interest. Synthesis of corresponding chloro analog 9-(3-chlorophenyl)-5-(2-hydroxyethyl)-6,9-dihydro-[1,3]dioxolo[4,5-g]furo[3,4-b]quinolin-8(5H)-one can also be seen in U.S. Pat. Appln. Publication No. 2017/0342086 (Kumar).

### Method:

Pharmacokinetics study: SU056 Plasma concentration was determined by mass quantification analysis using an Agilent 6490 Funnel triple quadrupole (QQQ) mass spectrometer equipped with an Agilent 1290 infinity II UHPLC. An analytical C18 column, ZORBAX C18 (Eclipse Plus, 2.1×50 mm, 1.8 μm particle size) was used. The mobile phase was composed of 60% water buffered with 0.1% formic acid and 4 mM ammonium formate and 40% acetonitrile buffered with 0.1% formic acid. The flow rate of mobile phase was set at 0.4 mL/min and column temperature was adjusted at 30° C. The electrospray ionization source was operated in positive ion mode. Mass spectrometer parameters were optimized as: source temperature 550° C., nebulizer gas (nitrogen) 20 psi, ion spray (IS) voltage 5000 V, collision energy 21 V. Multiple reaction monitoring (MRM) method was used for the detection of SU056 and an internal standard (IS), 4-(2-hydroxyethyl)-6-methoxy-9-phenyl-4,9-dihydrofuro[3,4-b]quinolin-1(3H)-one, a similar analogue of SU056. The precursor ion [M+H]+ and product ion for SU056 were monitored at m/z 370.0 and m/z 274.2, respectively. And the precursor ion [M+H]+ and product ion for IS were monitored at m/z 338.1 and m/z 260, respectively. A calibration curve was generated using known concentrations of SU056 and IS, and this curve was used to calculate unknown concentrations of SU056 in the plasma at different time points. SU056 was injected at the dose of 20 mg/kg to each mouse at time zero intraperitonially. Blood was collected retro-orbitally at 5, 15, 30, 60, 120, 240 and 360 minutes after injection. Blood plasma was separated via centrifugation at 7,000 RPM for 10 minutes. A 5 μL plasma was taken from each sample and mixed with a 10 μL of IS solution and 990 μL of MS-grade acetonitrile then vortexed for 30 seconds followed by a 5 minutes incubation at RT. Mixture was centrifuged at 11,000 RPM for 15 min at 4° C. and supernatant was collected and further cleaned by re-centrifugation. Each protein-free plasma fraction (n=3) was used to determine the concentration of SU056 using the HPLC/MS MRM method as described above.

TABLE 1
Most significant (average p < 0.05) processes found to be enriched
after 12 hours treatment with SU056 according to GSEA using the KEGG
pathway database. Proteins shown in bold were enriched in each pathway.

| KEGG Pathway Increase | p-value | Proteins in pathway detected (bold are enriched) |
|---|---|---|
| Apoptosis | 0.01 | AIFM1, PRKAR2A, PIK3CG, CAPN1, CAPN2, CYCS, DFFA, |
| | | PRKAR1A |
| Alanine, aspartate, and | 0.02 | ADSS, GLUD1, ADSL, GFPT1, GOT1, GOT2, GLS, CAD |
| glutamate metabolism | | |
| RNA degradation | 0.03 | PAPOLB, LSM2, DDX6, ENO2, HSPA9, HSPD1, ENO1 |
| Arginine and proline | 0.04 | ALDH18A1, GLUD1, GOT1, SMS, OAT, ALDH9A1, GOT2, |
| metabolism | | P4HA1, CKB, SRM, GLS, LAP3, ALDH7A1 |
| FcεRI signaling pathway | 0.01 | VAV2, MAPK14, PIK3CG, RAC1, GRB2 |
| T-cell receptor signaling | 0.02 | VAV2, MAPK14, PIK3CG, RHOA, GRB2, CDC42, PAK2 |
| pathway | | |
| Natural killer cell mediated | 0.03 | VAV2, PTPN11, PIK3CG, RAC1, GRB2, ICAM1 |
| cytotoxicity | | |
| Epithelial cell signaling in | 0.04 | MAPK14, ATP6V1G1, PTPN11, RAC1, EGFR, ATP6V1A, |
| helicobacter pylori infection | | CDC42 |
| FcyR mediated phagocytosis | 0.05 | MARCKSL1, VAV2, ARPC4, PIK3CG, RAC1, CFL2, VASP, |
| | | ARPC2, ARPC3, ARPC5, GSN, ARPC1B, MARCKS, CDC42, |
| | | CFL1, DNM2, ARPC5L |
| Spliceosome | 0.02 | SF3A3, SF3B2, CWC15, SF3B1, SF3A2, SF3B3, SNRPA1, |
| | | RBM25, SRSF7, SNW1, EFTUD2, DDX42, SF3B4, HNRNPA3, |
| | | PCBP1, ACIN1, SRSF10, NHP2L1, PRPF40A, CDC5L, |
| | | DDX39B, SF3A1, U2SURP, U2AF1, PPIL1, HSPA8, RBM8A, |
| | | SNRNP200, RBMX, PRPF8, SNRPD2, HNRNPA1, CTNNBL1, |
| | | MAGOH, SRSF1, HSPA1B, SRSF4, HNRNPM, HSPA6, |
| | | SRSF3, HNRNPK, SNRPC, SNRNP70, SRSF9, PUF60, DDX5, |
| | | PRPF19, DHX15, HNRNPC, HNRNPU, SNRPD3, U2AF2, |
| | | SNRPB, LSM2, EIF4A3, SRSF2 |

Cell lines: Human ovarian cancer (OC) OVCAR3, OVCAR4, OVCAR5, OVCAR8, and SKOV-3 cell lines were obtained from the NCI cell line repository (DTP). SH-SY5Y and N27 cell lines were obtained from Dr. Manish Chamoli (Buck Institute, CA, USA). ID8 and Luciferase-tagged ID8 cells were obtained from Dr. Erinn Rankin (Stanford University, CA, USA). Scrambled control (SC), YBX1 knockdown (1 & 2) and mCherry-YBX1 OVCAR8 cell lines were created using lentiviral based transduction and selected using puromycin resistance followed by cell shorting. All OVCAR cells were maintained in RPMI-1640 (Corning, USA; #10-040-CV) supplemented with 10% FBS (Corning, USA; #35-015-CV) and 1% Antibiotic-Antimycotic solution (Gibco, USA; Ser. No. 15/240,062). SKOV3 and ID8 cells were maintained in DMEM media (Corning, USA; #10-013-CV) supplemented with 10% FBS and 1% Antibiotic-Antimycotic solution. All cells were maintained at 37° C. and 5% CO₂. SH-SY5Y and N27 cells were maintained in DMEM/F12 media (Hyclone, #SH30525.01) supplemented with 10% FBS and 1% Antibiotic-Antimycotic solution. All cells were maintained at 37° C. and 5% CO₂. OVCAR8 cells were tagged with Luciferase using lentiviral vector-based plasmid pLenti PGK Blast V5-LUC (w528-1) (gifted by Eric Campeau & Paul Kaufman, Addgene #19166) and selected using blasticidin. Luciferase-tagged cells were maintained as described above.

Cell viability assay: Cell viability was assessed using the standard MTT assay protocol. In brief, 5,000 cells were plated in each well of 96 well plates (Corning-Costar, #3598) and allowed to attach for 24 h. Cells were treated with a respective concentration of compounds for respective time points. A stock solution of each compound was prepared in DMSO. DMSO concentration was kept constant and maintained below 0.1%. After each incubation, 50 μL of 0.5 mg/ml MTT solution prepared in 1×PBS was added to each well followed by 1 h incubation at 37° C. and 5% CO₂. MTT solution and media were then removed, and MTT formazan crystals were dissolved in 100 μL of DMSO per well. Absorbance at 570 nm for each well was recorded using a multimode plate reader to quantitate MTT crystallization. Each absorbance value was normalized to controls and converted into percent cell viability.

Clonogenic assay: 300 suspended OC cells were plated in each well of 12 well plates (Corning-Costar, #3598) and incubated for 24 h for attachment. The media was replaced after 24 h with media containing a respective concentration of the test compound and incubated for 5-8 days until visible colonies appeared in vehicle-treated wells. Cells were then washed and fixed with 2% paraformaldehyde, followed by washing and staining with 0.5% Crystal violet for 1 h. Cells were de-stained using DI water and allowed to dry. Colonies were counted under the microscope at 100× magnification.

Cell cycle analysis: 30,000 OC cells were plated in each well of 12 well plates and incubated for 24 h. Cells were treated with a respective concentration of test compounds for 6 h. Both live and dead cells were collected via trypsinization and cell pellets were fixed with 70% ethanol. Fixed cells were stained using propidium iodide (PI) cocktail (80 μg/mL RNase A and 50 μg/mL PI in saponin-EDTA solution) and incubated at 4° C. overnight. Each sample was analyzed using a Guava® easyCyte™ Flow Cytometer (Millipore, Burlington, MA). FlowJo software calculated the % cells in each cell cycle phase.

Apoptotic cell death assay: Cells were plated and treated as described in cell cycle analysis assay above. Treated cells were incubated for 24 h and both live and dead cells were collected. Cells were stained with Annexin V and PI using FITC Annexin V apoptosis detection kit (BD Pharmingen, San Jose, CA) by following the manufacturer's protocol. Guava® easyCyte™ Flow Cytometer was used to analyze the stained cells.

Cell migration assay: 1×10⁵ cells were plated in 60 mm cell culture dishes (Corning-Falcon, #353002) and incubated for 24 h. Cells were treated with a respective concentration of each compound for 12 h. Cells were trypsinized and collected in a conical tube for each plate. Pellets of cells were resuspended, and live cells were counted using a hemocytometer and trypan blue staining. 40,000 live cells were then plated in the upper chamber of 8-micron transwell (Corning-Falcon, #353097) with 0.2% FBS media. The lower chamber contained 10% FBS complete media. Cells were incubated at 37° C. and 5% CO₂ for 16 h. Each transwell was washed, swiped for non-migrated cells and fixed using 75% ethanol. Fixed transwells were stained with 0.5% crystal violet for 1 h. De-stained transwell membranes were cut and mounted on a slide using DPX mounting media. Migrated cells were counted under the microscope at 100× magnification.

Cellular Thermal Shift Assay (CETSA): The assay was performed as previously described (Savitski et al., 2014). Briefly, OVCAR8 cells were treated with vehicle (DMSO) or SU056 (2.5 μM) at 70-80% confluency for 1.5 h. Cells were harvested and washed with 1×PBS twice. Cells were pelleted and resuspended in PBS. 10 different PCR tubes with 1×10⁶ cells/tube (in 100 μL PBS) were prepared for both the groups. Tubes were exposed to respective temperatures (37, 41, 44, 47, 50, 53, 56, 59, 63, 67° C.) for 3 min using a thermal cycler (Bio-Rad, CA, USA) followed by 2 min incubation at room temperature. Each tube was snap-frozen in liquid nitrogen. Cells were lysed using freeze/thaw cycle, and soluble and insoluble fractions were separated by centrifugation at 14000 RPM for 30 min at 4° C. An equal amount of soluble fraction for each temperature of both groups was labeled with Tandem Mass Tag (TMT) using manufacturer's protocol (TMT10plex™ Isobaric Label Reagent Set, #90110, Thermo Fisher Scientific, Waltham, MA). TMT labeled samples were analyzed using LC-MS/MS in triplicates, as previously described by our group (Going et al., 2018).

CETSA protein quantification, normalization, curve fitting, estimation of slope, and melting point and statistical analysis: Proteins were quantified from individual peptide spectra by a sum-based bootstrap algorithm using each corresponding TMT reporter ion intensity after correcting for isotope impurities using MaxQuant (Cox and Mann, 2008) in both vehicle and SU056 treated samples. In each sample, the lowest temperature was used as reference to calculate the log 2 ratio of signal of the soluble fraction in each temperature, and each signal was compared with the highest temperature to estimate the percentage of signal lost in the soluble fraction. A fitting model of the curves to the S-curves with the Boltzmann Equation was used to describe the statistical behavior in a thermodynamic system not in a state of equilibrium, like a denaturalization curve, using the same principles as previously described (Savitski et al., 2014). To this end, the sum of squares difference between the original and Bolztmann adjusted curve was minimized using a brute-force algorithm with R, solving the Bolztmann equation to calculate the slope of the melting curve and the half-value and the temperature in which half of the protein has been denatured. The melting point differences between the fitted curves with correlations above 0.75 and p-value less than 0.01 were considered as specific interactors of the drug.

Immunoblotting: Once 70% confluency of OC cells plated in 100 mm cell culture was obtained, dishes were treated with a respective concentration of SU056 for a respective time. At the end of treatment, cells were collected and lysed using M-PER™ lysis solution (Thermo Scientific, #78503), supplemented with Halt protease and phosphatase inhibitor cocktail (Thermo Scientific, #78440). An equal amount (40-60 μg) of proteins was resolved using 8%/10%/12% SDS-PAGE gel electrophoresis. Proteins were then transferred onto a PVDF membrane (Bio-Rad, #162-01277). Blocked membranes were incubated with respective primary antibody solution prepared in 5% nonfat milk TPBS at 4° C. overnight with gentle rocking. Primary antibody-probed membranes were washed and probed with respective HRP-conjugated secondary antibody. Proteins were detected using Immobilon® Crescendo Western HRP Substrate (Millipore, Germany) and visualized on an IVIS Lumina Imaging System (Perkin Elmer, Waltham, MA). The following primary and secondary antibodies were used: YB-1 (Cell signaling technology (CST), #8475; 1:2000), TMSB10 (R&D Systems, #AF6429; 1:2000), SUMO2/3 (CST, #4971; 1:1000), PSMB2 (Bethyl Laboratories, #A303817AT; 1:1000), MDR1 (CST, #13978; 1:2000), CD44 (CST, #37259; 1:1000), c-Myc (Novusbio, #NB600-302SS; 1:2000), CDK2 (CST, #2546; 1:2000), CDC25A (CST, #3652; 1:1000), Cyclin E (CST, #4132; 1:2000), Bax (CST, #5023; 1:1000), Bcl-2 (CST, #2876; 1:1000), GBP1 (Abnova, #H00002633-PW1, 1:2000), β-actin (Novusbio, #NB600-501 SS; 1:10000), anti-mouse IgG HRP-linked antibody (CST, #7076, 1:5000), and anti-rabbit IgG HRP-linked antibody (CST, #7074, 1:5000).

Total YB-1 sandwich ELISA: Total YB-1 protein level was analyzed using PathScan® Total YB1 Sandwich ELISA Kit (Cell Signaling, #12543) by following the manufacturer's protocol.

Multidrug Resistance Assay: The effect of SU056 on Multidrug Resistance of different OC cells was assayed by using the Multidrug Resistance Assay Kit (Fluorometric MDR Assay) (Sigma-Aldrich, #MAK161), following the manufacturer's protocol.

CD44 ELISA: The effect of SU056 on CD44 expression of different OC cells was assayed by using Human CD44 ELISA Kit (Colorimetric) (Novusbio, #NBP1-86819), following the manufacturer's protocol

Pulldown assay using biotinylated SU056: protein pulldown assay was performed using biotinylated SU056. 1. Pulldown from cells: OVCAR8 cells were treated with 2.5 μM biotinylated SU056 for 1.5 h. Treated cells were collected and lysed using M-PER™ lysis solution (Thermo Scientific, #78503), supplemented with Halt protease and phosphatase inhibitor cocktail (Thermo Scientific, #78440). 300 μg protein was incubated with magnetic conjugate streptavidin bead (CST, #5947) at 4° C. on rocker overnight. 2. Pulldown from cell lysates: OVCAR8 cells were collected and lysed using M-PER™ lysis solution (Thermo Scientific, #78503), supplemented with Halt protease and phosphatase inhibitor cocktail (Thermo Scientific, #78440). 1000 μg protein was incubated with 10 μM biotinylated SU056 at 4° C. on rocker overnight followed by overnight incubation with magnetic conjugate streptavidin bead (CST, #5947) at 4° C. After both the pulldown, biotin-streptavidin conjugates were pulldown and washed using magnetic rack. After three washing, beads were resuspended in 2×SDS sample buffer followed by heating at 90-100° C. for 5 min. Samples were resolved and probed with GBP1 (Abnova, #H00002633-PW1, 1:2000) and YB-1 (CST, #8475; 1:2000) as described above in immunoblotting. Samples of protein lysates from OVCAR8 with and without biotinylated SU056 (2.5 μM) treatment (input), samples from pulldown using only biotin and only streptavidin beads were also resoled as experimental controls.

Surface Plasmon Resonance (SPR): experiments were performed using a Biacore T200 (GE Healthcare) instrument at 25° C. The His-tagged (N-Terminal) YB-1 protein (Novusbio, #NBP2-30101) was captured via the His-tag on an NTA chip (GE Healthcare) and immobilized through amine coupling amine coupling chemistry using N-hydroxysuccinimide (NHS) and N′-(3-dimethylaminopropyl) carbodiimide hydrochloride (EDC) (GE Healthcare). All small molecule (SU093 and SU056) analysis experiments were performed in PBS (10 mM Phosphate buffer, 2.7 mM KCl, 0.137 NaCl) running buffer pH adjusted to yield pH 7.4 when supplemented with 5% DMSO. To investigate binding of the compounds or at 6 different (1-100 μM) concentrations prepared from a 10 mM stock solution, with a final concentration of 5% DMSO were injected over the different immobilized ligands at a flow rate of 30 μl/min for either 75 or 120 seconds with a regeneration time of 600 seconds. After each injection the flow delivery system was washed with 50% DMSO. DMSO solvent correction curves were generated by injecting the running buffers with serial concentrations of DMSO ranging from 4.5 to 5.8%. All data were corrected for non-specific binding by subtracting the signal measured in a control cell lacking immobilized ligand.

Global proteome profiling: OVCAR8 cells were treated with vehicle or SU056 (2.5 μM) for 12 h. Cells were washed and lysed (100 mM triethylammonium bicarbonate (TEAB, Thermo Fisher Scientific) with 1% sodium dodecyl sulfate (SDS)). Samples were processed and labeled with TMT using manufacturer's protocol (TMTsixplex™ Isobaric Label Reagent Set, #90061, Thermo Fisher Scientific, Waltham, MA). Samples were analyzed using LC-MS/MS in triplicates as previously described by our group (Going et al., 2018).

Combination index calculation: The combination index (CI) for paclitaxel and SU093 or SU056 was calculated using the Chou-Talalay Method (Chou, 2010). CI values were computed via CampuSyn software. CI<1, CI=1, and CI>1 indicate the synergistic, additive and antagonistic effect of the combination.

Paclitaxel efflux assay: OC cells were treated with 5 nM Oregon Green™ 488-conjugated paclitaxel (Molecular Probes, #P22310) for 1 h. Cells were washed and incubated in phenol red-free DMEM media supplemented with 10% FBS. After each respective time point (30, 60, 120, 180 minutes), the media was collected and centrifuged to remove floating cells. The fluorescence intensity of efflux paclitaxel in media was read at Ex 496 and Em 524 using a multimode plate reader.

Spheroid culture: OC cells at a density of 100 per well were plated in an ultra-low attachment 24 well plate (Corning, #3473) in MEGM media (Lonza, #CC-3150) supplemented with hEGF, insulin, hydrocortisone BPE, and 2-mercaptoethanol. Cells were treated with each respective compound and incubated for 6-8 days. The number of spheroids was counted under the microscope at 40× magnification. 5 different fields from each well were imaged at 100× magnification.

In vivo xenograft model and drug efficacy study: All animal experiments were reviewed and approved by the Animal Care and Use Committee of Stanford University, CA, USA. Luciferase tagged ID8 (2×10⁶) or OVCAR8 (5×10⁶) cells were implanted into the right flank of 6-7-week-old female C57BL/6 mice and NOD/SCID mice, respectively. Respective treatment began after tumors grew to 100-200 mm³ diameters. The ID8 syngeneic mice model was treated with a vehicle (30% PEG-300 in saline), 20 mg/kg SU093, and 20 mg/kg SU056 intraperitoneally (IP) daily for 42 days. At the end of treatment, blood was collected from each mouse and analyzed for liver toxicity parameters, including Alanine Aminotransferase (ALT), Aspartate Aminotransferase (AST), and Alkaline Phosphatase (ALKP). OVCAR8 xenograft mice model was treated with vehicle (30% PEG-300 in saline), 5 mg/kg paclitaxel (once a week), 10 mg/kg SU056 (daily), a combination of paclitaxel (5 mg/kg, once a week) and SU056 (10 mg/kg, daily) for 4 weeks. Mice from both studies were euthanized, the tumor and different organs were collected and fixed in neutral buffered formalin and further processed for immunohistochemical analysis.

Immunohistochemistry: Fixed tumors and organs were embedded in paraffin. Each block was cut in 5 μm sections and fixed on poly-L lysine-coated slides. Paraffin sections were deparaffinized and rehydrated. Antigen retrieved sections were incubated with primary antibody followed by HRP conjugated secondary antibody and Dab staining using ImmPACT™ DAB kit (Vectorlabs, CA). Counterstained sections were dehydrated and mounted using VectaMount™ (Vector labs). Biotinylated horse anti-mouse IgG (Vector Labs, CA) and horse anti-rabbit IgG (Vector Labs, CA) were used as secondary antibodies. The following primary antibodies were used: YB-1 (Cell signaling, #8475; 1:100) and anti-MDR1 (Cell signaling, #13978; 1:500) and Ki67 (Biolegend, #350502; 1:500).

Statistical analysis: Each set of data was analyzed for its statistical significance using GraphPad Prism 6 software. Each result is represented in Mean±SD. P-values are denoted using * as follows: *P≤0.05, **P≤0.01, ***P≤0.001.

Presence of expressed YBX1 protein in cancer cells referenced herein may be identified by methods known in the art. Non-limiting examples include the YBX1 ELISA Kit available from antibodies-online Inc, Jones Blvd 321, Limerick, PA 19464 (Catalog No. ABIN6975583).

In immunohistochemical methods for human testing, YB1 (D2B12) Rabbit mAb #8475 (available from Cell Signal Technology online at cellsignal.com/products/primary-antibodies/yb1-d2b12-rabbit-mab/8475?site-search-type=Products&N=4294956287&Ntt=ybx1 &fromPage=plp.

### Significance

New chemotherapeutic strategies are urgently needed to improve the treatment of cancer, where late diagnosis and high risk of relapse collude with treatment resistance (TR) to cause high mortality despite intensive chemotherapy treatment regimens. Y box binding protein 1 (YB1 or YBX1) is the multifunction protein which binds to the DNA and RNA and is associated with tumor progression and the emergence of TR. YB-1 plays the important role in transcription, translation and RNA stabilization of various oncogenic proteins. Role of YB-1 is very well established in various cancers but there is no small molecule inhibitor available/reported so far. Herein, we report a novel azopodophyllotoxin (AzP) derivative, SU056, that inhibits disease progression via YB1 inhibition. This first-in class YB-1 inhibitor potently inhibits the ovarian cancer (OC) cell proliferation and resistance to apoptosis and arrests the cells in G1 phase. This treatment leads to an enrichment of proteins associated with apoptosis and RNA degradation pathway and downregulates the spliceosome pathway. In vivo, SU056 independently restrains ovarian cancer progression and exerts a synergistic effect with paclitaxel to further reduce disease progression with no liver toxicity. Moreover, in vitro mechanistic studies showed delayed disease progression via inhibition of drug efflux and multi-drug resistance 1 (MDR1) and significantly low neural toxicity as compared to Etoposide. These data suggest that YB-1 inhibitor may be an effective strategy to reduce OC progression, TR and decrease patient mortality.

### Data Availability

The mass spectrometry proteomics data have been deposited to the PRIDE Archive (http://www.ebi.ac.uk/pride/archive/) via the PRIDE partner repository with the data set identifier PXD022332.

### REFERENCES

AGARWAL et al., 2003. Ovarian cancer: strategies for overcoming resistance to chemotherapy. Nat Rev Cancer, 3, 502-16.
ANDREOLI et al., 2014. Identification of the first inhibitor of the GBP1:PIM1 interaction. Implications for the development of a new class of anticancer agents against paclitaxel resistant cancer cells. J Med Chem, 57, 7916-32.
BARGOU et al., 1997. Nuclear localization and increased levels of transcription factor YB-1 in primary human breast cancers are associated with intrinsic MDR1 gene expression. Nat Med, 3, 447-50.
BLAGOSKLONNY et al., 1999. Molecular effects of paclitaxel: myths and reality (a critical review). Int J Cancer, 83, 151-6.
BLATTNER et al., 2000. UV-Induced stabilization of c-fos and other short-lived mRNAs. Mol Cell Biol, 20, 3616-25.
CHANSKY et al., 2001. Oncogenic TLS/ERG and EWS/Fli-1 fusion proteins inhibit RNA splicing mediated by YB-1 protein. Cancer Res, 61, 3586-90.
CHEN et al., 2017. The STAT3-miRNA-92-Wnt Signaling Pathway Regulates Spheroid Formation and Malignant Progression in Ovarian Cancer. Cancer Res, 77, 1955-1967.
CHOU, T. C. 2010. Drug combination studies and their synergy quantification using the Chou-Talalay method. Cancer Res, 70, 440-6.
CHRISTIE et al., 2017. Acquired chemotherapy resistance in ovarian cancer. Ann Oncol, 28, viii13-viii15.
COX et al., 2008. MaxQuant enables high peptide identification rates, individualized p.p.b.-range mass accuracies and proteome-wide protein quantification. Nat Biotechnol, 26, 1367-72.
DE DONATO et al., 2012. Class III beta-tubulin and the cytoskeletal gateway for drug resistance in ovarian cancer. J Cell Physiol, 227, 1034-41.
DIDIER et al., 1988. Characterization of the cDNA encoding a protein binding to the major histocompatibility complex class II Y box. Proc Natl Acad Sci USA, 85, 7322-6.
DIETL, J. 2014. Revisiting the pathogenesis of ovarian cancer: the central role of the fallopian tube. Arch Gynecol Obstet, 289, 241-6.
EL-NAGGAR et al., 2015. Translational Activation of HIF1alpha by YB-1 Promotes Sarcoma Metastasis. Cancer Cell, 27, 682-97.
EVDOKIMOVA et al., 2001. The major mRNA-associated protein YB-1 is a potent 5′ cap-dependent mRNA stabilizer. EMBO J, 20, 5491-502.
EVDOKIMOVA et al., 2009. Translational activation of snail1 and other developmentally regulated transcription factors by YB-1 promotes an epithelial-mesenchymal transition. Cancer Cell, 15, 402-15.
FLETCHER et al., 2010. ABC transporters in cancer: more than just drug efflux pumps. Nat Rev Cancer, 10, 147-56.
FRANZESE et al., 2019. PARP inhibitors in ovarian cancer. Cancer Treat Rev, 73, 1-9.
FRIED et al., 1954. 9α-Fluoro Derivatives of Cortisone and Hydrocortisone. Journal of the American Chemical Society, 76, 1455-1456.
GENOVESE et al., 2017. Not only P-glycoprotein: Amplification of the ABCB1-containing chromosome region 7q21 confers multidrug resistance upon cancer cells by coordinated overexpression of an assortment of resistance-related proteins. Drug Resist Updat, 32, 23-46.
GILLIS et al., 2015. Applications of Fluorine in Medicinal Chemistry. Journal of Medicinal Chemistry, 58, 8315-8359.
GOING et al., 2018. Quantitative Proteomic Profiling Reveals Key Pathways in the Anticancer Action of Methoxychalcone Derivatives in Triple Negative Breast Cancer. J Proteome Res, 17, 3574-3585.
GOODARZI et al., 2015. Endogenous tRNA-Derived Fragments Suppress Breast Cancer Progression via YBX1 Displacement. Cell, 161, 790-802.
GOTTESMAN et al., 1993. Biochemistry of multidrug resistance mediated by the multidrug transporter. Annu Rev Biochem, 62, 385-427.
GOTTESMAN et al., 2015. The Role of Multidrug Resistance Efflux Pumps in Cancer: Revisiting a JNCI Publication Exploring Expression of the MDR1 (P-glycoprotein) Gene. J Natl Cancer Inst, 107.
GRAUMANN et al., 1998. A superfamily of proteins that contain the cold-shock domain. Trends Biochem Sci, 23, 286-90.
HARADA et al., 2014. YB-1 promotes transcription of cyclin D1 in human non-small-cell lung cancers. Genes Cells, 19, 504-16.
HORWITZ et al., 1986. Taxol: mechanisms of action and resistance. Ann NY Acad Sci, 466, 733-44.
JAYSON et al., 2014. Ovarian cancer. Lancet, 384, 1376-88.
JUNG et al., 2018. Clinical Importance of Myc Family Oncogene Aberrations in Epithelial Ovarian Cancer. JNCI Cancer Spectrum, 2.
JUNG et al., 2017. A Myc Activity Signature Predicts Poor Clinical Outcomes in Myc-Associated Cancers. Cancer Res, 77, 971-981.
KANG et al., 2013. Role of focal adhesion kinase in regulating YB-1-mediated paclitaxel resistance in ovarian cancer. J Natl Cancer Inst, 105, 1485-95.
KRETOV et al., 2019. YB-1, an abundant core mRNA-binding protein, has the capacity to form an RNA nucleoprotein filament: a structural analysis. Nucleic Acids Res, 47, 3127-3141.
KUWANO et al., 2004. The role of nuclear Y-box binding protein 1 as a global marker in drug resistance. Mol Cancer Ther, 3, 1485-92.
KUZNETSOVA et al., 2012. Synthesis and Biological Evaluation of Novel 3′-Difluorovinyl Taxoids. J Fluor Chem, 143, 177-188.
LAIRD-OFFRINGA et al., 1990. Poly(A) tail shortening is the translation-dependent step in c-myc mRNA degradation. Mol Cell Biol, 10, 6132-40.
LHOMME et al., 2008. Phase III study of valspodar (PSC 833) combined with paclitaxel and carboplatin compared with paclitaxel and carboplatin alone in patients with stage IV or suboptimally debulked stage III epithelial ovarian cancer or primary peritoneal cancer. J Clin Oncol, 26, 2674-82.
LI et al., 2009. Efflux-mediated drug resistance in bacteria: an update. Drugs, 69 (12), 1555-623.
LIM et al., 2018. Targeting metabolic flexibility via angiopoietin-like 4 protein sensitizes metastatic cancer cells to chemotherapy drugs. Mol Cancer, 17, 152.
LYABIN et al., 2014. YB-1 protein: functions and regulation. Wiley Interdiscip Rev RNA, 5, 95-110.
MAITY et al., 2011. Class III beta-tubulin (TUBB3): more than a biomarker in solid tumors? Curr Mol Med, 11, 726-31.
MATSUMOTO et al., P. 1998. Gene regulation by Y-box proteins: coupling control of transcription and translation. Trends Cell Biol, 8, 318-23.
MO et al., 2016. Human Helicase RECQL4 Drives Cisplatin Resistance in Gastric Cancer by Activating an AKT-YB1-MDR1 Signaling Pathway. Cancer Res, 76, 3057-66.
RUFF et al., 2009. A randomized, placebo-controlled, double-blind phase 2 study of docetaxel compared to docetaxel plus zosuquidar (LY335979) in women with metastatic or locally recurrent breast cancer who have received one prior chemotherapy regimen. Cancer Chemother Pharmacol, 64, 763-8.
SAUPE et al., 2015. Differential expression of the multidrug resistance 1 (MDR1) protein in prostate cancer cells is independent from anticancer drug treatment and Y box binding protein 1 (YB-1) activity. World J Urol, 33, 1481-6.
SAVITSKI et al., 2014. Tracking cancer drugs in living cells by thermal profiling of the proteome. Science, 346, 1255784.
SEIFTER, E. J. 1997. Cancer: Principles and Practice of Oncology, 5th Edition Vincent T. DeVita, Jr., Samuel Hellman, Steven A. Rosenberg, eds. Philadelphia:Lippincott-Raven Publishers, 1997.3125 pp., illus. ISBN 0-397-51573-4. JNCI: Journal of the National Cancer Institute, 89, 353-353.
SHIOTA et al., 2014. Targeting ribosomal S6 kinases/Y-box binding protein-1 signaling improves cellular sensitivity to taxane in prostate cancer. Prostate, 74, 829-38.
SINGH, A. & SETTLEMAN, J. 2010. EMT, cancer stem cells and drug resistance: an emerging axis of evil in the war on cancer. Oncogene, 29, 4741-51.
SOBOCAN et al., 2020. The Communication Between the PI3K/AKT/mTOR Pathway and Y-box Binding Protein-1 in Gynecological Cancer. Cancers (Basel), 12.
STICKELER et al., 2001. The RNA binding protein YB-1 binds A/C-rich exon enhancers and stimulates splicing of the CD44 alternative exon v4. EMBO J, 20, 3821-30.
SUN et al., 2015. Integrative transcriptomics-based identification of cryptic drivers of taxol-resistance genes in ovarian carcinoma cells: Analysis of the androgen receptor. Oncotarget, 6, 27065-82.
WARTENBERG et al., 1998. Development of an intrinsic P-glycoprotein-mediated doxorubicin resistance in quiescent cell layers of large, multicellular prostate tumor spheroids. Int J Cancer, 75, 855-63.
WARTENBERG et al., 2005. Regulation of the multidrug resistance transporter P-glycoprotein in multicellular prostate tumor spheroids by hyperthermia and reactive oxygen species. Int J Cancer, 113, 229-40.
WU et al., 2007. YB-1 is a Transcription/Translation Factor that Orchestrates the Oncogenome by Hardwiring Signal Transduction to Gene Expression. Transl Oncogenomics, 2, 49-65.
WU et al., 2014. Cell fate factor DACH1 represses YB-1-mediated oncogenic transcription and translation. Cancer Res, 74, 829-39.
ZENG et al., 2018. Targeting MYC dependency in ovarian cancer through inhibition of CDK7 and CDK12/13. Elife, 7.
ZHAO et al., 2016. YBX1 regulates tumor growth via CDC25a pathway in human lung adenocarcinoma. Oncotarget, 7, 82139-82157.

FIG. 1A) Lead optimization of SU093 to obtain SU056. FIG. 1B) IC₅₀ values of SU093 and SU056 on various ovarian cancer cells. Clonogenic survival of OC cells treated with SU093 or SU056. 300-500 ovarian cancer cells were plated/well of 12 well plate and allow to attach for 24 h. Cells were treated with SU093 and SU056 and incubated further for 7 days. Each well was stained with crystal violet and colonies were counted under 1 OX microscope. FIG. 1C) Representative colony formation from respective wells. FIG. 1D) Number of colonies formed after SU093 and SU056 treatment. FIG. 1E) % inhibition values of etoposide, SU093, and SU056 treatment at 10 μM concentration for 48 h in neuronal (SH-SY5Y, N27) and HEK293 cells. FIG. 1F) Cell cycle distribution of propidium iodide (PI)-stained OVCAR8, SKOV3, and ID8 cells. Effects of SU093 and SU056 on cell cycle distribution showing G1 phase arrest after 12 h treatment. FIG. 1G) Effects of SU093 and SU056 on apoptotic cell death analyzed by Annexin-FITC staining. Both the compounds induce apoptotic cell death in ovarian cancer cells after 24 h treatment. FIG. 1H) Cell migration assay. Imaging of cells after 16 h through Boyden chambers showed that treatment by SU093 and SU056 significantly reduces the cell migration property of ovarian cancer cells. Data are shown as mean±SD of triplicate samples. *P<0.05, significantly different compared with respective controls by one-way ANOVA followed by Dunnett's test.

FIGS. 2A-2F. SU093 and SU056 inhibit mice ovarian ID-8 tumor xenograft growth in C₅₇BL/6 mice. Mice were subcutaneously injected with ID-8 cells mixed with Matrigel in 1:1 ratio and the drug treatment started when tumors reached 100 mm³. Mice were intraperitoneally (IP) injected with either vehicle (30% PEG300 in saline) or 20 mg/kg SU093 or SU056 for 42 days daily. FIG. 2A) Representative images of mice after 42 days of drug treatment showed tumor regression compared to control. FIG. 2B) Tumor volume/mouse as a function of time. Data shown are mean±SD from 5 mice in each group. *P<0.05, compared with respective control. FIG. 2C) Tumor weight/mouse at the end of the study. Data shown are mean±SD from 5 mice in each group. *P<0.05, **P<0.01, ***P<0.001, compared with respective control. FIG. 2D) Liver toxicity parameters at the end of 42 days showing no significant difference between control, SU093, and SU056. FIGS. 2E-2F) Lung metastasis assay. FIG. 2E) H&E staining of the lung (red arrow indicates the metastasis from ID8 xenograft). Scale bar, 250 μm. FIG. 2F) No. of lung metastatic nodules. Data shown are mean±SD from 5 mice in each group. *P<0.05, **P<0.01, ***P<0.001, compared with respective control.

FIGS. 3A-3F: Target identification using Cellular Thermal Shift Assay (CETSA). Differential profiling of SU056 on the thermal proteome profile of OVCAR8 cells. OVCAR8 cells were treated with DMSO or SU056 (2.5 μM) for 1.5 h. Cells were collected and 10⁶ cells in each PCR tube incubated at different temperatures (37, 41, 44, 47, 50, 53, 56, 59, 63, 67° C.). Cells were lysed and an equal quantity of soluble protein was labeled with TMT, followed by LC-MS/MS analysis. FIG. 3A) Heat map representation of the thermal stability of 804 soluble proteins in ovarian cancer cells treated with vehicle-DMSO (left) and SU056 (right). FIG. 3B) Density distributions of protein Tm values calculated in SU056 treated cells (red) and vehicle cells (blue). FIG. 3C) Density distributions of Tm shifts between SU056 and vehicle treatment. FIG. 3D) A scatter plot of Tm calculated in SU056 and vehicle treatment. Proteins that passed the significant values (p-value<0.01, RSQ>0.7) and identification criteria are highlighted in red. FIG. 3E) Melting curves for identified top six proteins (YB-1, TMSB10, SUMO-2, PSMB2, TMSB4X, and CALM3) with and without SU056 treatment. FIG. 3F) Change in melting temperature (Tm) of the top six proteins upon SU056 treatment.

FIGS. 4A-4E: SU056 inhibits YB-1. FIG. 4A) OVCAR8 cells were treated with SU056 (1, 2.5 and 5 μM) for 12 h and total cell lysates were prepared as described in the ‘Methods’ section. SDS-PAGE and Western blot analysis was performed for the top three targets identified by CETSA (YB-1, TMSB10, SUMO-2 and PSMB2). Membranes were stripped and re-probed with an anti-beta-actin antibody to ensure equal protein loading. FIG. 4B) Immunohistochemistry of tumor samples from ID8 tumor xenograft study from control and SU056-a related group for YB-1 and MDR1 expression. Subset images are at 20× magnification: scale bar, 50 μm. FIGS. 4C-4E) Effect of SU056 on YB-1 and its associated proteins in different ovarian cancer cell lines. FIG. 4C) Respective OC cells were treated with 2.5 μM SU056 for 12 h and YB-1 & CD44 expression and multidrug resistance activity was measured as described in the ‘method’ section. The % inhibition was calculated and compared to the control for the respective cell line. FIG. 4D) YB-1 50% inhibitory concentration (IC50) of SU056 for OC cell lines was determined after 12 h of treatment using PathScan® Total YB1 Sandwich ELISA Kit. FIG. 4E) YB-1 inhibition time kinetics study for SU056 effect on OC cell lines. Cells were treated with SU056 (1, 2.5 and 5 μM) for 3, 6, 12, and 24 h. YB-1 was assayed using PathScan® Total YB1 Sandwich ELISA Kit.

FIGS. 5A-5G: SU056 physically interact with YB-1. FIG. 5A) Structure of biotinylated SU056. FIG. 5B) Pulldown assay using biotinylated SU056. Pulldown were carried out from OVCAR8 cells and OVCAR8 cell lysates as described in ‘Methods’ secession. Both the pulldowns were run in duplicate. FIGS. 5C-5D) Representative sensograms for FIG. 5C) SU093 and FIG. 5D) SU056. His-tagged YB-1 protein was immobilized on NTA chip and different concentration of SU093 and SU056 (1-100 μM) were tested for physical interaction as described in ‘Methods’ secession. FIGS. 5E-5G) Cellular effect of SU056 is dependent on YB-1 expression. OVCAR8 cells were stably express with Scrambled control (SC), YBX1 shRNA1 and YBX1 shRNA2 using lentiviral vector. FIG. 5E) Western blot analysis was performed to conform the YB-1 expression in transduced cells. FIG. 5F) 500 cells (SC, shRNA1, shRNA2) were plated/well of 12 well plate and allow to attach for 24 h. Transduced cells were treated with SU056 and incubated further for 7 days. Each well was stained with crystal violet and colonies were counted under 1 OX microscope. Representative colony formation from respective wells. FIG. 5G) IC₅₀ values of SU056 on different transduced OVCAR8 cells expressing SC, YBX1 shRNA1, YBX1 shRNA2.

FIGS. 6A-6F: SU056 modulates the YB-1 associated proteins and pathways. FIG. 6A) Cycloheximide chase assay (CHX) to determine the effect of SU056 on YB-1 protein stability. Data are shown as mean±SD of triplicate samples. *P<0.05, significantly different compared with respective controls by one-way ANOVA followed by Dunnett's test. FIG. 6B) Total cell lysates were prepared as described in the ‘Methods’ section. SDS-PAGE and Western blot analysis was performed for YB-1, cell cycle, and apoptosis-associated markers. Membranes were stripped and re-probed with an anti-beta-actin antibody to ensure equal protein loading. FIGS. 6C-6F) GSEA was performed on the proteomics results to determine the enrichment of KEGG pathways upon treatment on the OVCAR8 cell line with SU056. FIG. 6C) Enrichment plot of pathways modulated by SU056 treatment. FIGS. 6D-6E) Enrichment in the Apoptosis and RNA degradation pathway was observed in proteins that increase in abundance upon treatment with SU056. FIG. 6F) Enrichment in the Spliceosome pathways was observed in proteins that decrease in abundance upon treatment with SU056.

FIGS. 7A-7I: SU056 treatment sensitizes the ovarian cancer cell for taxane treatment. FIG. 7A) Sensitizing effects of SU056 on the viability of OVCAR8 and SKOV3 cells in combination with paclitaxel treatment. Cells treated with 0.1, 0.5, and 1 μM SU056 followed by 0.1, 0.5 and 1 nM paclitaxel treatment for 48 h showed a synergistic cytotoxic effect. Combination index values for paclitaxel and SU056. FIG. 7B) Alexa Fluor-488-tagged paclitaxel efflux assay showed that SU056 cotreatment inhibits paclitaxel efflux. FIG. 7C) Immunoblotting of YB-1 and MDR1. OVCAR8 cells were treated with either vehicle (C), paclitaxel (0.5 nM, P), SU056 (0.5 μM, 56) and paclitaxel+SU056 (P+56) for 12 h and cell lysates were prepared. Proteins were resolved on SDS-PAGE gel and blotted for respective antibodies as described in the method section. Membranes were stripped and re-probed for loading control actin. Spheroid formation assay. 500 cells were cultured in ultra-low attachment plates and treated with each drug and their combinations. The cells incubated for 7 days to form spheroids. FIG. 7D) Microscopic images of spheroid at 1 OX magnification. Scale bar, 250 μm FIG. 7E) Spheroid formation was quantified after 7 days of incubation. Data are shown as mean±SD of triplicate samples. *P<0.05, **P<0.01, ***P<0.001, significantly different compared with respective controls by one-way ANOVA followed by Dunnett's test. FIGS. 7F-7I) Combination study of SU056 and paclitaxel on the OVCAR8 xenograft model. NOD-SCID female mice were subcutaneously injected with OVCAR8 cells mixed with Matrigel in 1:1 ratio. Drug treatment started when tumors grew to 200 mm³. Mice were intraperitoneally (IP) injected with either vehicle (30% PEG300 in saline) or 10 mg/kg SU056 daily and/or 5 mg/kg Paclitaxel once a week for 28 days (4 weeks). FIG. 7F) Representative images of mice after 28 days of drug treatment showing tumor regression compared to control. FIG. 7G) Tumor volume/mouse as a function of time. FIG. 7H) tumor weight/mouse at the end of the study. FIG. 7I) Immunohistochemistry staining. Tumor sections were stained with Ki67, and slides were scored for K167 staining. Data shown are mean±SD from 5 mice in each group. *P<0.05, **P<0.01, ***P<0.001 compared with respective control.

FIGS. 8A-8C: A) Validation of CETSA results using western blot analysis. Cell lysates from control and SU056 treated cells exposed at 37 and 53° C. temperature were analyzed for expression of YB-1, TMSB10 and PSMB2. FIGS. 8B-8C) SU056 treatment inhibit the expression of YB-1 in OVCAR8 cells. OVCAR8 cells stably expressing with mCheery tagged YB-1 were used for these assays. FIG. 8B) Cells were imaged using confocal microscope for the mCherry-YB1 after 3 h treatment of SU056 (2.5 & 5 μM) at 10×magnification. FIG. 8C) 2500 cells were plated in 96 well plate (black well clear bottom) and treated with SU056 (1-5 μM) for 3 and 6 h. Relative fluorescence intensity was measured as using multimode plate reader. Data was normalized with % viable cells and presented in percentage compare to non-treated cells. Data shown are mean±SD from 5 replicates in each group. *P<0.05 compared with respective control.

FIG. 9: Sensitizing effects of SU056 on the viability of OVCAR8 and SKOV3 cells in combination with paclitaxel treatment. Cells treated with 0.1, 0.5 and 1 μM SU056 followed by 0.1, 0.5 and 1 nM paclitaxel treatment for 48 h showed synergistic cytotoxic effect.

FIG. 10: Pharmacokinetics of SU056. Determination of SU056 plasma concentration as a function of time (min) after administration of the drug intraperitonially at a dose of 20 mg/kg (n=3). After injection plasma was collected at respective time point and was analyzed using LC MS/MS as described in method.

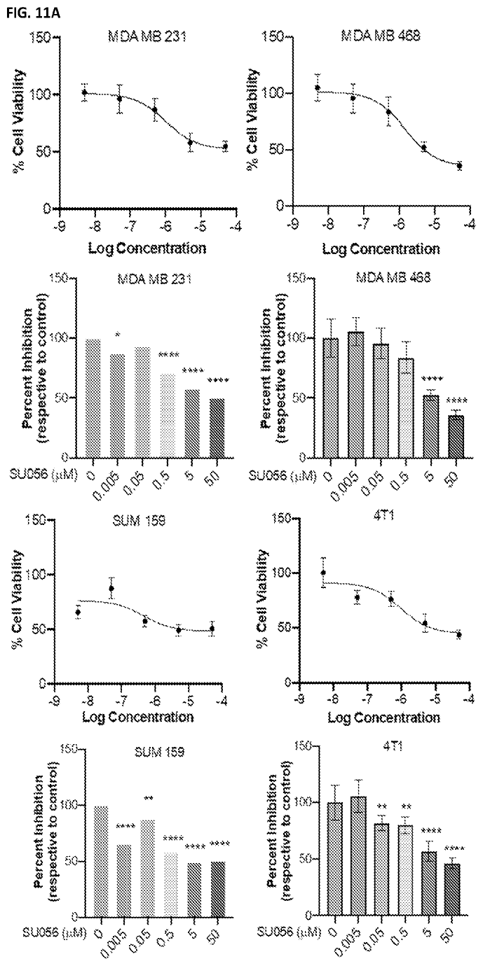

FIG. 11A). The growth inhibitory effect of SU056 was evaluated using MTT assay. TNBC cell lines (MDA-MB-231, MDA-MB-468, SUM159, 4T1, E0771 and EMT6) were plated in 96 well plate. Next day, treated with vehicle (DMSO) alone or 0.005-50 μM of SU056 in fresh medium. After 48 hr of treatments, cell viability was measured using MTT assay. FIG. 11B). A total of 300-600 cells were plated per well of a 12-well plate and allowed to attach for 24 hr. Next day, cells were treated with SU056 and incubated further to 7-10 days. Each well stained with crystal violet and colonies were counted under a 10×microscope. Representative colony formations from respective wells of cell. FIG. 11C) Number of colonies formed after SU056 treatment. FIG. 11D) Effect of SU056 on cell cycle distribution in TNBC cells. MDA-MB-231, MDA-MB-468 and SUM 159 cells were treated with vehicle or SU056 for 12 and 24 hr. At the end of treatment, cells were collected and analysed using flow cytometry. SU056 treatment showing G2/M phase arrest. SU056 induces level of phosphor-Histone H3 in cells at 12 and 24 hr.

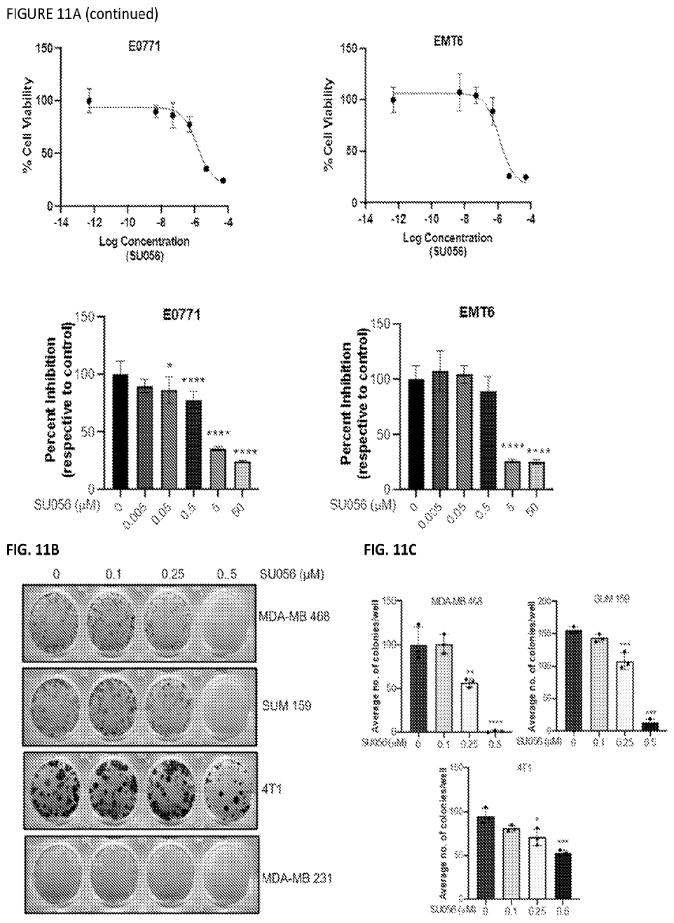

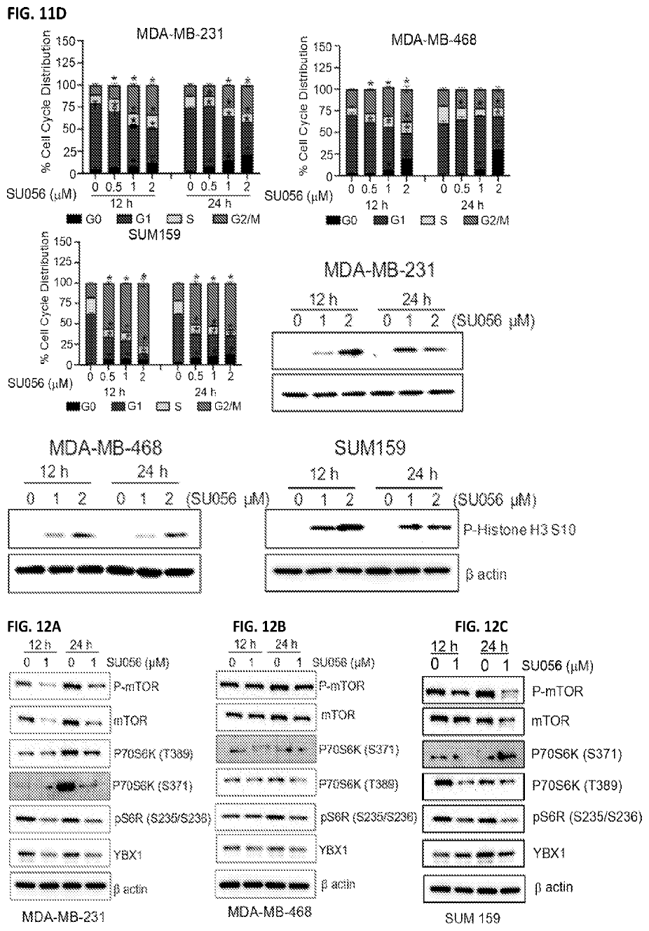

FIGS. 12A-12C. TNBC cells were treated for 12 and 24 hr and total cell lysate were prepared. SDS-PAGE and western blot analyses were performed for translation associated molecules. Beta-actin were probed to ensure equal protein loading. SU056 treatment inhibited the protein translation associated molecules among all TNBC cells FIG. 12A) MDA-MB-231, FIG. 12B) MDA-MB-468 and FIG. 12C) SUM 159.

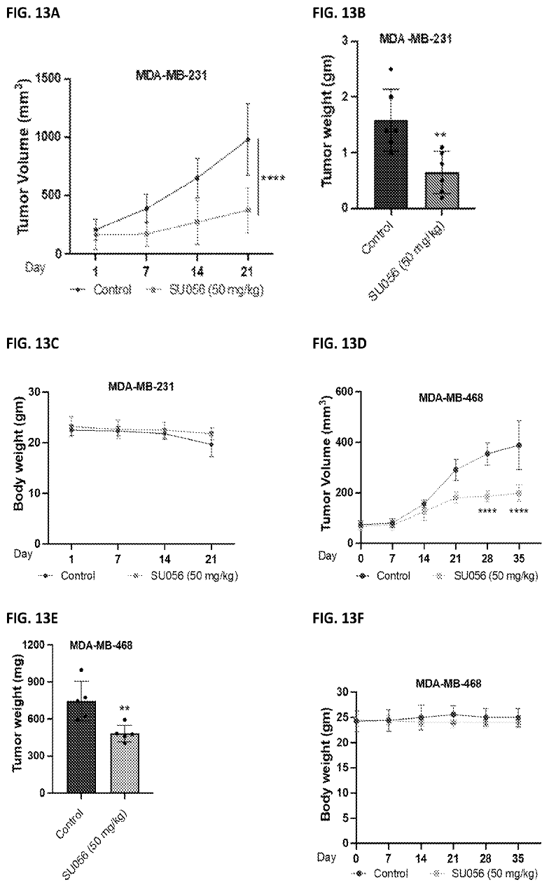

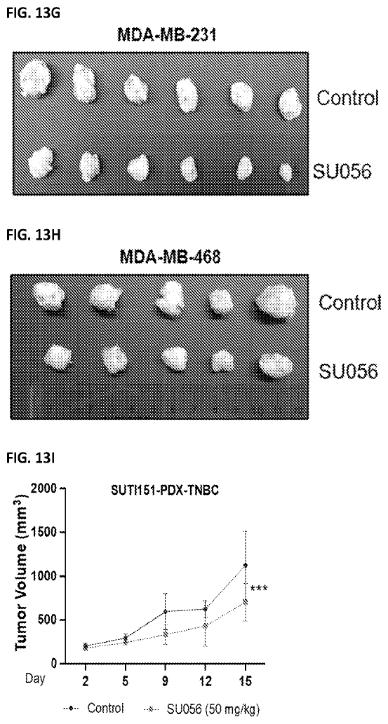

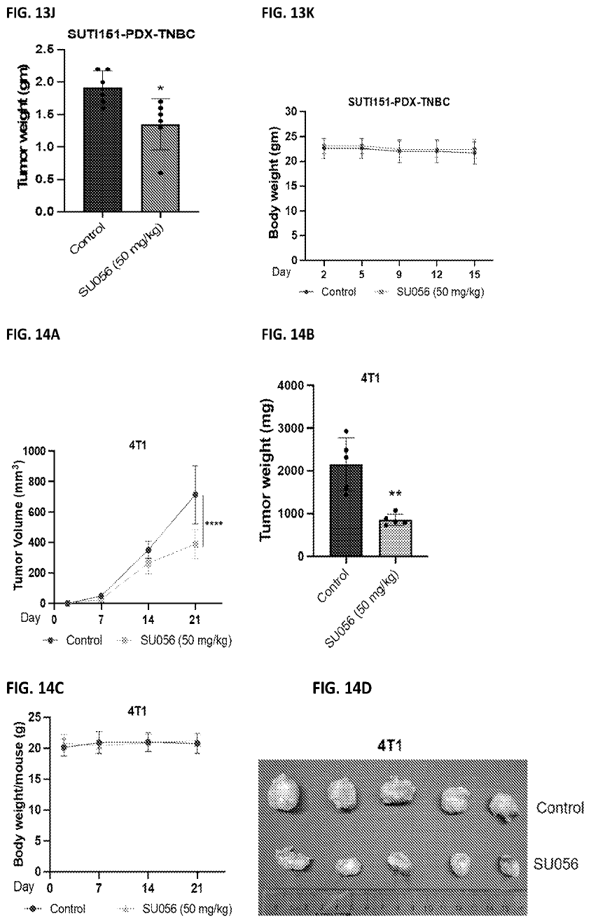

FIGS. 13A-13I. SU056 inhibit tumor xenograft of TNBC models. Mice were subcutaneously injected with MDA-MB-231 (2×10⁶ cells), MDA-MB-468 (5×10⁶ cells) and patient derived xenograft SUT1151-PDX (2×10⁶ cells) and drug treatment started when tumor reached 100 mm³. Mice were given either vehicle (40% PEG in saline) or SU056 (50 mg/kg) through oral route using oral gavage. FIG. 13A) Tumor volume (MDA-MB-231) as a function of time. FIG. 13B) Tumor weight (MDA-MB-231) at the end of the study. FIG. 13C) Body weight (MDA-MB-231) as a function of time. FIG. 13D) Tumor volume (MDA-MB-468) as a function of time. FIG. 13E) Tumor weight (MDA-MB-468) at the end of the study. FIG. 13F) Body weight (MDA-MB-468) as a function of time. FIG. 13G) Representative images of tumor (MDA-MB-231) at the end of study. FIG. 13H) Representative images of tumor (MDA-MB-468) at the end of study. FIG. 13I) Tumor volume (SUT1151-PDX) as a function of time. FIG. 13B) Tumor weight (SUT1151-PDX) at the end of the study. FIG. 13C) Body weight (SUT1151-PDX) as a function of time.

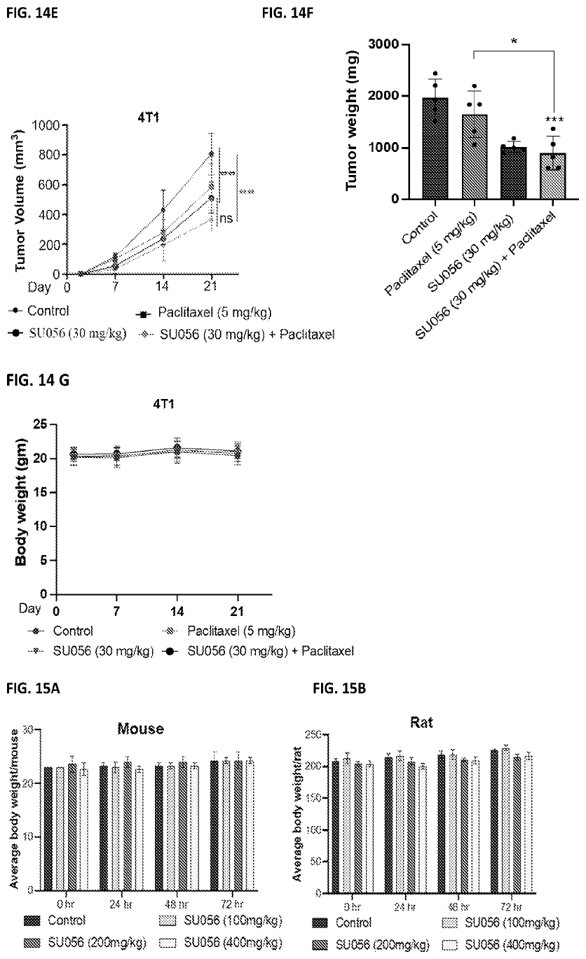

FIGS. 14A-14G SU056 inhibit 4T1 tumor xenograft in BALB/c. Mice were subcutaneously injected with 4T1 cells and drug treatment started after three day of implantation. Mice were given either vehicle (40% PEG in saline) or SU056 (50 mg/kg) through oral route using oral gavage. FIG. 14A) Tumor volume (4T1) as a function of time. FIG. 14B) Tumor weight (4T1) at the end of the study. FIG. 14C) Body weight (4T1) as a function of time. FIG. 14D) Representative images of tumor (4T1) at the end of study. Combination of Paclitaxel and SU056 in 4T1 xenograft model. FIG. 14E) Tumor volume (4T1) as a function of time. FIG. 14F) Tumor weight (4T1) at the end of the study. FIG. 14G) Body weight (4T1) as a function of time.

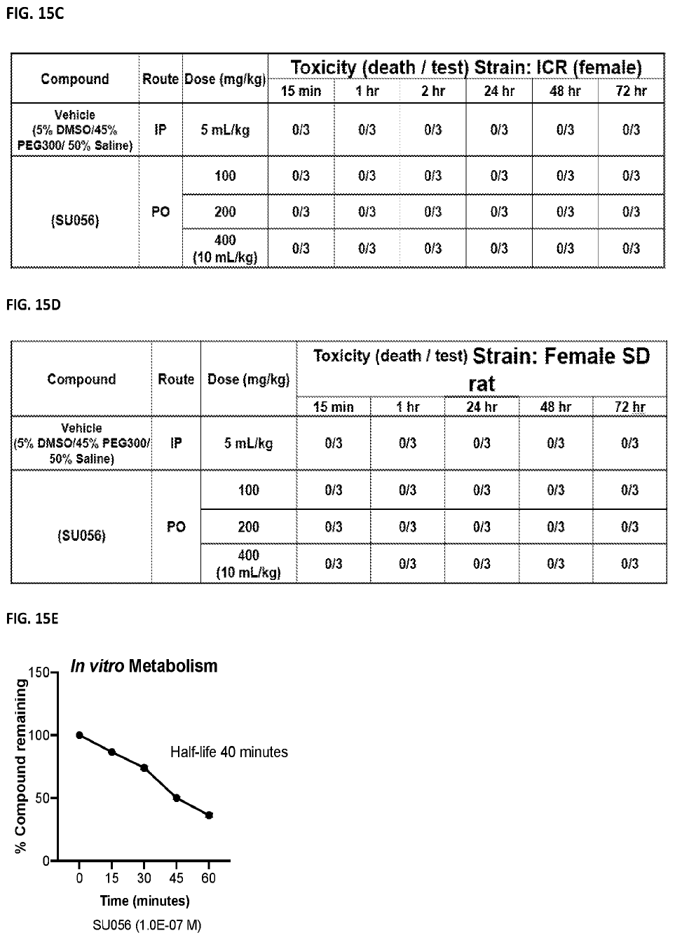

FIGS. 15A-15E. SU056 treatment is well tolerated in mice and rat. SU056 treatment at increasing concentrations dose not challenge the dietary behavior as reflected by change in body weight FIG. 15A) Mice and FIG. 15B) Rat. SU056 treatment of different concentration did not cause death among FIG. 15C) mice and FIG. 15D) Rat. Liver microsomes were used for the purpose of metabolic profiling. FIG. 15E) SU056 had a mean half-life of 40 minutes.

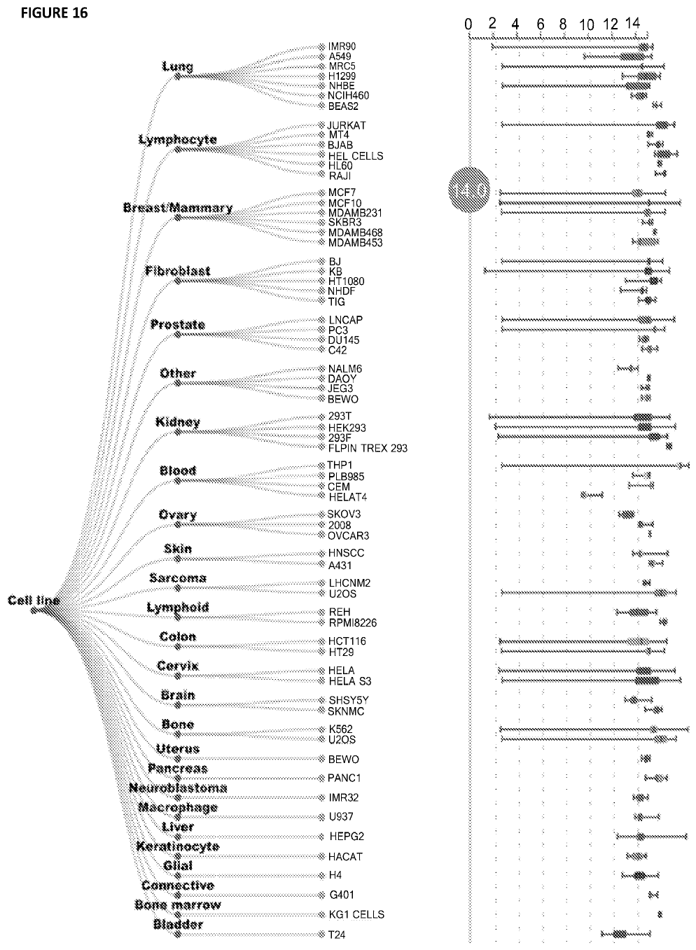

FIG. 16 depicts reference-normalized expression levels of YBX1 for the cancer cell lines: a) Lung—IMR90, A549, MRC5, H1299, NHBE, NCIH460, and BEAS2; b) Lymphocyte—JURKAT, MT4, BJAB, HEL CELLS, HL60, and RAJI; c) Breast/Mammary—MCF7, MCF10, MDAMB231, SKBR3, MDAMB468, and MDAMB453; d) Fibroblast—BJ, KB, HT1080, NHDF, and TIG; e) Prostate—LNCAP, PC3, DU145, and C42; f) Other—NALM6, DAOY, JEG3, and BEWO; g) Kidney—293T, HEK293, 293F, and FLPIN TREX 293; h) Blood—THP1, PLB985, CEM, and HELAT4; i) Ovary—SKOV3, 2008, and OVCAR3; j) Skin—HNSCC and A431; k) Sarcoma—LHCNM2 and U2OS; l) Lymphoid—REH and RPM18226; m) Colon—HCT116 and HT29; n) Cervix—HELA and HELA S3; o) Brain—SHSY5Y and SKNMC; p) Bone—K562 and U20S; q) Uterus—BEWO; r) Pancreas—PANC1; s) Neuroblastoma—IMR32; t) Macrophage U937; u) Liver—HEPG2; v) Keratinocyte—HACAT; w) Glial—H4; x) Connective—G401; y) Bone Marrow—KG1 CELLS; and a) Bladder—T24. This information is provided online by the Ma'ayan Lab Data Coordination and Integration Center at the Mount Sinai Center for Bioinformatics and may be accessed at: maayanlab.cloud/archs4/gene/YBX1 #correlation.

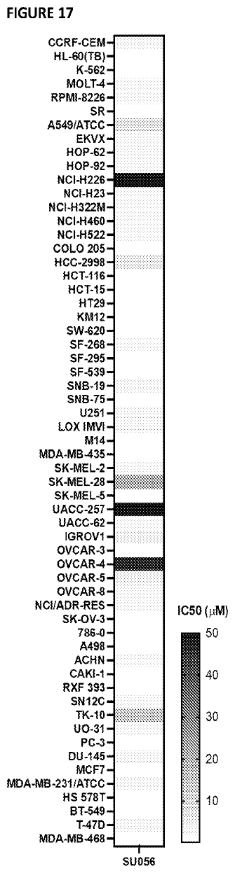

FIG. 17: Assay of SU056 in NCI-60 cell line panel. SU056 was assayed against the NCI-60 cell line panel at five dose (100 to 0.01 μM) for 48 hours, providing the IC50 values in the table below. Growth inhibition was measured and displayed as a heatmap from no inhibition (black), to growth inhibition (white), as seen in FIG. 17.

NCI60-IC50 values

| Cell Lines | IC50 (μM) |
|---|---|
| CCRF-CEM | 2.37 |
| HL-60(TB) | 1.26 |
| K-562 | 0.353 |
| MOLT-4 | 3.02 |
| RPMI-8226 | 2.28 |
| SR | 0.294 |
| A549/ATCC | 8.74 |
| EKVX | 3.03 |
| HOP-62 | 2.19 |
| HOP-92 | 3.1 |
| NCI-H226 | 50 |
| NCI-H23 | 3.96 |
| NCI-H322M | 4.01 |
| NCI-H460 | 2.08 |
| NCI-H522 | 2.59 |
| COLO 205 | 1.46 |
| HCC-2998 | 7.06 |
| HCT-116 | 1.53 |
| HCT-15 | 1.08 |
| HT29 | 1.52 |
| KM12 | 1.48 |
| SW-620 | 1.65 |
| SF-268 | 3.2 |
| SF-295 | 1.2 |
| SF-539 | 1.13 |
| SNB-19 | 3 |
| SNB-75 | 0.82 |
| U251 | 2.5 |
| LOX IMVI | 2.35 |
| M14 | 0.357 |
| MDA-MB-435 | 0.187 |
| SK-MEL-2 | 2.46 |
| SK-MEL-28 | 20.3 |
| SK-MEL-5 | 1.26 |
| UACC-257 | 50 |
| UACC-62 | 2.25 |
| IGROV1 | 4.19 |
| OVCAR-3 | 1.27 |
| OVCAR-4 | 46.8 |
| OVCAR-5 | 4.33 |
| OVCAR-8 | 3.18 |
| NCI/ADR-RES | 2.02 |
| SK-OV-3 | 1.73 |
| 786-0 | 1.86 |
| A498 | 1.11 |
| ACHN | 3.03 |
| CAKI-1 | 1.24 |
| RXF 393 | 1.51 |
| SN12C | 3.05 |
| TK-10 | 15.6 |
| UO-31 | 2.88 |
| PC-3 | 1.46 |
| DU-145 | 2.71 |
| MCF7 | 1.44 |
| MDA-MB-231/ATCC | 3.09 |
| HS 578T | 1.42 |
| BT-549 | 1.46 |
| T-47D | 2.18 |
| MDA-MB-468 | 1.12 |

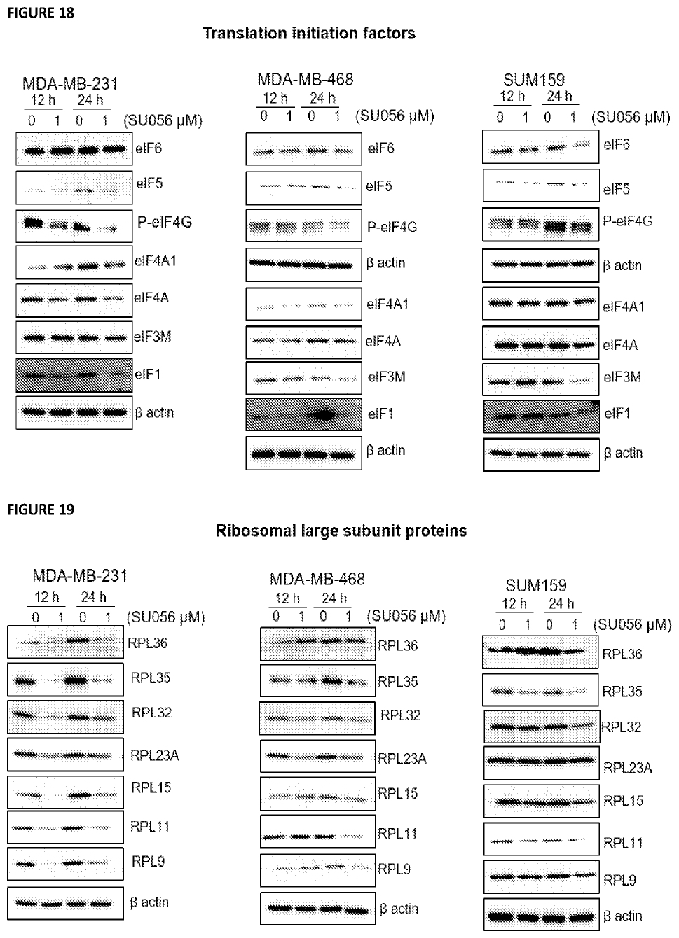

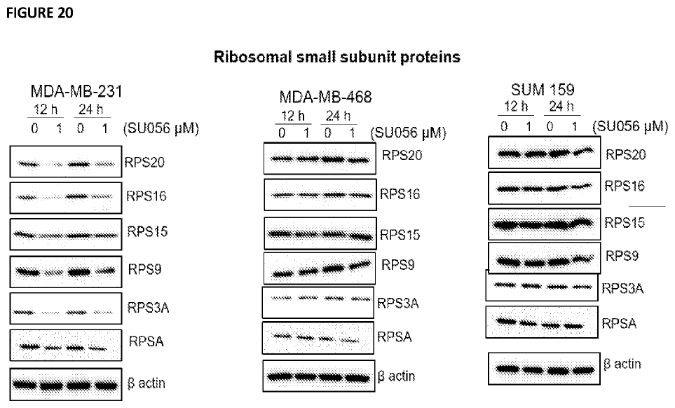

In each of FIGS. 18, 19, and 20: TNBC cells were treated for 12 and 24 hr and total cell lysate were prepared. SDS-PAGE and western blot analyses were performed for translation associated molecules. Beta-actin were probed to ensure equal protein loading. SU056 treatment inhibited the translation initiation factors in TNBC cells.

### STAR Methods

### Key Resources Table

| | | |
|---|---|---|
| Reagent or Resource | Source | Identifier |

Antibodies

| | | |
|---|---|---|
| YB-1 | Cell signaling technology (CST) | #8475 |
| TMSB10 | R&D Systems | #AF6429 |
| SUMO2/3 | CST | #4971 |
| PSMB2 | Bethyl Laboratories | #A303817AT |
| MDR1 | CST | #13978 |
| CD44 | CST | #37259 |
| GBP1 | Abnova | #H00002633-PW1 |
| c-Myc | Novesbio | # NB600-302SS |
| CDK2 | CST | #2546 |
| CDC25A | CST | #3652 |
| Cyclin E | CST | #4132 |
| Bax | CST | #5023 |
| Bcl-2 | CST | #2876 |
| β-actin | Novusbio | #NB600-501SS |
| Ki67 | Biolegend | #350502 |
| Anti-mouse IgG HRP-linked antibody | CST | #7076 |
| Anti-rabbit IgG HRP-linked antibody | CST | #7074 |

Recombinant Proteins

| | | |
|---|---|---|
| Human YB-1 protein (His Tag) | Novusbio | # NBP2-30101 |

Chemicals

| | | |
|---|---|---|
| RPMI-1640 | Corning | #10-040-CV |
| DMEM | Corning | #10-013-CV |
| DMEM/F12 | Hyclone | #SH30525.01 |
| MEGM media | Lonza | #CC-3150 |
| FBS | Corning | #35-015-CV |
| Antibiotic-Antimycotic solution | Gibco | 15240062 |
| M-PER™ lysis solution | Thermo Scientific | #78503 |
| Halt protease and phosphatase | Thermo Scientific | #78440 |
| inhibitor cocktail | | |
| Magnetic conjugate streptavidin bead | CST | #5947 |
| Oregon Green™ 488-conjugated paclitaxel | Molecular Probes | # P22310 |

Cell Lines

| | |
|---|---|
| OVCAR3 | NCI cell line repository (DTP) |
| OVCAR4 | NCI cell line repository (DTP) |
| OVCAR5 | NCI cell line repository (DTP) |
| OVCAR8 | NCI cell line repository (DTP) |
| SKOV3 | NCI cell line repository (DTP) |
| SH-SY5Y | Dr. Manish Chamoli (Buck |
| | Institute, CA, USA) |
| ID8 | Dr. Erinn Rankin (Stanford |
| | University, CA, USA) |

Assay kits

| | | |
|---|---|---|
| Tandem Mass Tag (TMT) | Thermo Fisher Scientific | # 90110, #90061 |
| PathScan® Total YB1 Sandwich ELISA Kit | CST | #12543 |
| Multidrug Resistance Assay Kit | Sigma-Aldrich | #MAK161 |
| Human CD44 ELISA Kit (Colorimetric) | Novusbio | #NBP1-86819 |

Recombinant DNA

| | | |
|---|---|---|
| pLenti PGK Blast V5-LUC (w528-1) | Eric Campeau & Paul Kaufman, | Addgene # 19166 |
| | Addgene | |
| pLV[Exp]-Puro- | VectorBuilder Inc | |
| EF1A>hYBX1[NM_004559.5]/mCherry | | |
| pLV[shRNA]-EGFP:T2A:Puro- | VectorBuilder Inc | |
| U6>hYBX1[shRNA#1] | | |
| shRNA sequence: | | |
| CCTGTTAATAAAGGTCTTAAA (SEQ ID | | |
| NO: 1) | | |
| pLV[shRNA]-EGFP:T2A:Puro- | VectorBuilder Inc | |
| U6>hYBX1[shRNA#2] | | |
| shRNA sequence: | | |
| CCAGTTCAAGGCAGTAAATAT (SEQ ID | | |
| NO: 2) | | |
| pLV[shRNA]-EGFP:T2A:Puro- | VectorBuilder Inc | |
| U6>Scramble[shRNA#1] | | |
| shRNA sequence: | | |
| CCTAAGGTTAAGTCGCCCTCG (SEQ ID | | |
| NO: 3) | | |

Human YB-1 protein (His Tag) (Novus Biological Catalog No. NBP2-30101) is a recombinant protein with a N-terminal His-tag and corresponding to the amino acids 1-324 of Human YB1. Source: E. coli. Gene: YBX1. Amino Acid Sequence:

(SEQ ID NO: 4)
MGSSHHHHHH SSGLVPRGSH MGSMSSEAET QQPPAAPPAA
PASLSAADTKP GTTGSGAGSG GPGGLETSAAP AGGDKKVIAT
KVLGTVKWFN VRNGYGFINR NDTKEDVFVH QTAIKKNNPR
KYLRSVGDGE TVEFDVVEGE KGAEAANVTG PGGVPVQGSK
YAADRNHYRR YPRRRGPPRN YQQNYQNSES GEKNEGSESA
PEGQAQQRRP YRRRFPPYY MRRPYGRRPQ YSNPPVQGEV
MEGADNQGAG EQGRPVRQNM YRGYRPRFRR GPPRQRQPRE
DGNEEDKENQ GDETQGQQPP QRRYRRNFNY RRRRPENPKP
QDGKETKAAD PPAENSSAPE AEQGGAE

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “0046-0079US.txt” created on or about Jun. 1, 2023, with a file size of 4 KB, contains the sequence listing for this application and is hereby incorporated by reference in its entirety.

What is claimed:

1. A method of inhibiting YB1 protein activity in a subject experiencing a cancer expressing YB1 protein, the method comprising administering to the subject in need thereof a pharmaceutically effective amount of a compound of Formula (I), 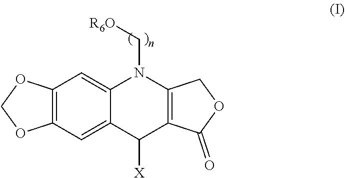
wherein:
X is selected from the group of: 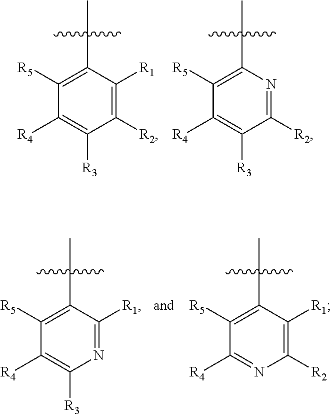
R₁, R₂, R₃, R₄, and R₅ are each independently selected in each instance from the group of H, F, C₁-C₄ fluoroalkyl, SF₅, Cl, Br, I, OH, C₁-C₄ alkyl, C₁-C₄ alkoxy, CN, NO₂, and OH;
n is an integer selected independently in each instance from the group of 1, 2, 3, 4, 5, and 6;
R₆ is selected from the group of H, C₁-C₆ alkyl, C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl; wherein the C₁-C₆ alkyl group is substituted with 0, 1, 2, 3, or 4 substituents selected from F, Cl, Br, I, OH, CN, NO₂, and OH; and the C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl groups are substituted with 0, 1, 2, 3, or 4 substituents selected from the group of C₁-C₃ alkyl, C₁-C₃ alkoxy, F, Cl, Br, I, OH, CN, NO₂, and OH;
with the proviso that at least one of R₁, R₂, R₃, R₄, and R₅ is selected from the group of F, C₁-C₄ fluoroalkyl, and SF₅; and
with the proviso that, when X is 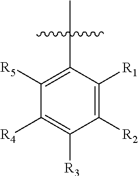
and R₂ is F or CF₃, then at least one of R₁, R₃, R₄, and R₅ is not H; or a pharmaceutically acceptable salt, co-crystal, ester, solvate, hydrate, isomer, tautomer, isotope, polymorph, or prodrug thereof.

2. The method of claim 1 wherein the compound of Formula (I) is a compound of Formula (Ia): 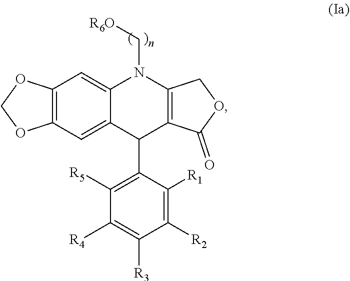
wherein n, R₁, R₂, R₃, R₄, R₅, and R₆ are as defined in claim 1; or a pharmaceutically acceptable salt, co-crystal, ester, solvate, hydrate, isomer, tautomer, isotope, polymorph, or prodrug thereof.

3. The method of claim 1 wherein the compound of Formula (I) is a compound of Formula (Ib): 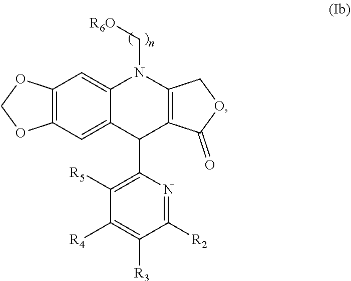
wherein n, R₂, R₃, R₄, R₅, and R₆ are as defined in claim 1; or a pharmaceutically acceptable salt, co-crystal, ester, solvate, hydrate, isomer, tautomer, isotope, polymorph, or prodrug thereof.

4. The method of claim 1 wherein the compound of Formula (I) is a compound of Formula (Ic): 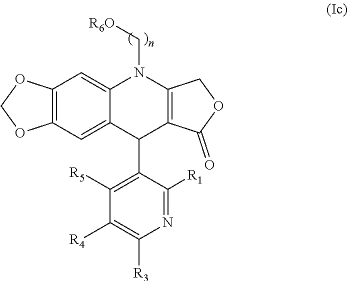
wherein n, R₁, R₃, R₄, R₅, and R₆ are as defined in claim 1; or a pharmaceutically acceptable salt, co-crystal, ester, solvate, hydrate, isomer, tautomer, isotope, polymorph, or prodrug thereof.

5. The method of claim 1 wherein the compound of Formula (I) is a compound of Formula (Id): 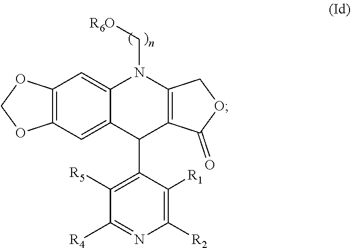
wherein n, R₁, R₂, R₄, R₅, and R₆ are as defined in claim 1; or a pharmaceutically acceptable salt, co-crystal, ester, solvate, hydrate, isomer, tautomer, isotope, polymorph, or prodrug thereof.

6. The method of claim 1 wherein the compound of Formula (I) is a compound of Formula (II): 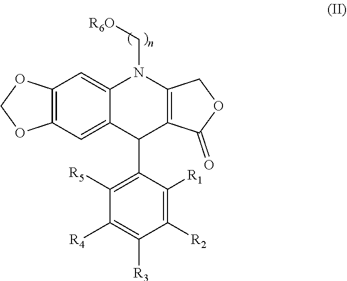
wherein:
R₁, R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH;
n is an integer selected independently in each instance from the group of 1, 2, 3, 4, 5, and 6;
R₆ is selected from the group of H, C₁-C₆ alkyl, C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl; wherein the C₁-C₆ alkyl group is substituted with 0, 1, 2, 3, or 4 substituents selected from F, Cl, Br, I, OH, CN, NO₂, and OH; and the C₃-C₆ cycloalkyl, —(CH₂)ₙ—C₃-C₆ cycloalkyl, 3-6-membered heterocycle, —(CH₂)ₙ-3-6-membered heterocycle, phenyl, and —(CH₂)ₙ-phenyl groups are substituted with 0, 1, 2, 3, or 4 substituents C₁-C₃ alkyl, C₁-C₃ alkoxy, F, Cl, Br, I, OH, CN, NO₂, and OH;
with the proviso that at least one of R₁, R₂, R₃, R₄, and R₅ is selected from the group of F, C₁-C₄ fluoroalkyl, and SF₅; and
with the proviso that, when R₂ is F or CF₃, then at least one of R₁, R₃, R₄, and R₅ is not H; or a pharmaceutically acceptable salt, co-crystal, ester, solvate, hydrate, isomer, tautomer, isotope, polymorph, or prodrug thereof.

7. The method of claim 1 wherein the compound of Formula (I) is a compound of Formula (III), 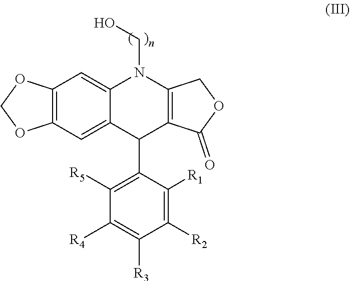
wherein: n is an integer selected independently in each instance from the group of 1, 2, 3, 4, 5, and 6;
R₁, R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH;
with the proviso that at least one of R₁, R₂, R₃, R₄, and R₅ is selected from the group of F, C₁-C₄ fluoroalkyl, and SF₅; and
with the proviso that, when R₂ is F or CF₃, then at least one of R₁, R₃, R₄, and R₅ is not H; or a pharmaceutically acceptable salt, co-crystal, ester, solvate, hydrate, isomer, tautomer, isotope, polymorph, or prodrug thereof.

8. The method of claim 7, wherein R₁ is selected from the group of F, C₁-C₄ fluoroalkyl, and SF₅; and R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH; or a pharmaceutically acceptable salt, co-crystal, ester, solvate, hydrate, isomer, tautomer, isotope, polymorph, or prodrug thereof.

9. The method of any of claims 7 and 8, wherein R₃ is selected from the group of F, C₁-C₄ fluoroalkyl, and SF₅; and R₁, R₂, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH; or a pharmaceutically acceptable salt, co-crystal, ester, solvate, hydrate, isomer, tautomer, isotope, polymorph, or prodrug thereof.

10. The method of any of claims 7, 8, and 9, wherein R₁, R₂, R₃, R₄, and R₅ are each independently selected from the group of H, F, C₁-C₄ fluoroalkyl, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are selected from the group of F, C₁-C₄ fluoroalkyl, and SF₅;
or a pharmaceutically acceptable salt, co-crystal, ester, solvate, hydrate, isomer, tautomer, isotope, polymorph, or prodrug thereof.

11. The method of any of claims 7, 8, 9, and 10, wherein R₁, R₂, R₃, R₄, and R₅ are each independently selected from the group of H, F, C₁-C₃ fluoroalkyl, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are selected from the group of F, C₁-C₃ fluoroalkyl;
or a pharmaceutically acceptable salt, co-crystal, ester, solvate, hydrate, isomer, tautomer, isotope, polymorph, or prodrug thereof.

12. The method of any of claims 7, 8, 9, 10, and 11, wherein R₁, R₂, R₃, R₄, and R₅ are each independently selected from the group of H, F, CF₃, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are selected from the group of F and CF₃;
or a pharmaceutically acceptable salt, co-crystal, ester, solvate, hydrate, isomer, tautomer, isotope, polymorph, or prodrug thereof.

13. The method of any of claims 7, 8, 9, 10, 11, and 12, wherein R₁, R₂, R₃, R₄, and R₅ are each independently selected from the group of H, F, CF₃, SF₅, Cl, Br, I, OH, C₁-C₄ alkyl, C₁-C₄ alkoxy, CN, NO₂, and OH;
with the proviso that at least two of R₁, R₂, R₃, R₄, and R₅ are F;
or a pharmaceutically acceptable salt, co-crystal, ester, solvate, hydrate, isomer, tautomer, isotope, polymorph, or prodrug thereof.

14. The method of any of claims 1-13, wherein n is an integer independently in each instance from the group of 1, 2, 3, 4, and 5; or a pharmaceutically acceptable salt, co-crystal, ester, solvate, hydrate, isomer, tautomer, isotope, polymorph, or prodrug thereof.

15. The method of any of claims 1-13, wherein n is an integer independently in each instance from the group of 1, 2, 3, and 4; or a pharmaceutically acceptable salt, co-crystal, ester, solvate, hydrate, isomer, tautomer, isotope, polymorph, or prodrug thereof.

16. The method of any of claims 1-13, wherein n is an integer independently in each instance from the group of 1, 2, and 3; or a pharmaceutically acceptable salt, co-crystal, ester, solvate, hydrate, isomer, tautomer, isotope, polymorph, or prodrug thereof.

17. The method of any of claims 1-13, wherein n is an integer independently in each instance from the group of 2 and 3; or a pharmaceutically acceptable salt, co-crystal, ester, solvate, hydrate, isomer, tautomer, isotope, polymorph, or prodrug thereof.

18. The method of any of claims 1-13, wherein n is 1; or a pharmaceutically acceptable salt, co-crystal, ester, solvate, hydrate, isomer, tautomer, isotope, polymorph, or prodrug thereof.

19. The method of any of claims 1-13, wherein n is 2; or a pharmaceutically acceptable salt, co-crystal, ester, solvate, hydrate, isomer, tautomer, isotope, polymorph, or prodrug thereof.

20. The method of any of claims 1-13, wherein n is 3; or a pharmaceutically acceptable salt, co-crystal, ester, solvate, hydrate, isomer, tautomer, isotope, polymorph, or prodrug thereof.

21. The method of any of claims 1-13, wherein n is 4; or a pharmaceutically acceptable salt, co-crystal, ester, solvate, hydrate, isomer, tautomer, isotope, polymorph, or prodrug thereof.

22. The method of any of claims 1-13, wherein n is 5; or a pharmaceutically acceptable salt, co-crystal, ester, solvate, hydrate, isomer, tautomer, isotope, polymorph, or prodrug thereof.

23. The method of any of claims 1-13, wherein n is 6; or a pharmaceutically acceptable salt, co-crystal, ester, solvate, hydrate, isomer, tautomer, isotope, polymorph, or prodrug thereof.

24. The method of any of claims 1-23, wherein R₆ is H; or a pharmaceutically acceptable salt, co-crystal, ester, solvate, hydrate, isomer, tautomer, isotope, polymorph, or prodrug thereof.

25. The method of any of claims 1-25, wherein R₆ is C₁-C₆ alkyl substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

26. The method of any of claims 1-25, wherein R₆ is C₃-C₆ cycloalkyl, wherein the C₃-C₆ cycloalkyl substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

27. The method of any of claims 1-25, wherein R₆ is —(CH₂)ₙ—C₃-C₆ cycloalkyl, wherein the C₃-C₆ cycloalkyl ring is substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

28. The method of any of claims 1-25, wherein R₆ is a 3-6-membered heterocycle, wherein the heterocycle ring is substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

29. The method of any of claims 1-25, wherein R₆ is a —(CH₂)ₙ-3-6-membered heterocycle, wherein the heterocycle ring is substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

30. The method of any of claims 1-25, wherein R₆ is phenyl, wherein the phenyl ring is substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

31. The method of any of claims 1-25, wherein R₆ is —(CH₂)ₙ-phenyl, wherein the phenyl ring is substituted with 0, 1, 2, 3, or 4 substituents selected from the group of C₁-C₃ alkyl, C₁-C₃ alkoxy, F, Cl, Br, I, OH, CN, NO₂, and OH; or a pharmaceutically acceptable salt, co-crystal, ester, solvate, hydrate, isomer, tautomer, isotope, polymorph, or prodrug thereof.

32. The method of any of claims 1-31, wherein two of R₁, R₂, R₃, R₄, and R₅, when present, are selected from the group of F, C₁-C₄ fluoroalkyl, and SF₅.

33. The method of any of claims 1-32, wherein two of R₁, R₂, R₃, R₄, and R₅, when present, are selected from the group of F, C₁-C₃ fluoroalkyl.

34. The method of any of claims 1-33, wherein two of R₁, R₂, R₃, R₄, and R₅, when present, are selected from the group of F and CF₃.

35. The method of any of claims 1-31, wherein three of R₁, R₂, R₃, R₄, and R₅, when present, are selected from the group of F and CF₃.

36. The method of any of claims 1-31, wherein four of R₁, R₂, R₃, R₄, and R₅, when present, are selected from the group of F and CF₃.

37. The method of any of claims 1-36, wherein two of R₁, R₂, R₃, R₄, and R₅, when present, are F.

38. The method of any of claims 1-36, wherein three of R₁, R₂, R₃, R₄, and R₅, when present, are F.

39. A pharmaceutical composition comprising a pharmaceutically effective amount of a compound of Formula (I) of any of claims 1-38, or a pharmaceutically acceptable salt, co-crystal, ester, solvate, hydrate, isomer, tautomer, isotope, polymorph, or prodrug thereof, and a pharmaceutically acceptable carrier or excipient.

40. The use of a compound of Formula (I) as described in any of claims 1-38, or a pharmaceutically acceptable salt, co-crystal, ester, solvate, hydrate, isomer, tautomer, isotope, polymorph, or prodrug thereof, in the preparation of a medicament for the treatment of a cancer expressing YB1 protein.

41. A method of inhibiting YB1 protein activity in a subject experiencing a cancer expressing the YB1 protein, the method comprising administering to the subject in need thereof a pharmaceutically effective amount of a compound of Formula (I) as described in any of claims 1-38, or a pharmaceutically acceptable salt, pharmaceutically acceptable co-crystal, ester, solvate, hydrate, isomer, tautomer, isotope, polymorph, or prodrug thereof.

41. A method of inhibiting YB1 protein activity in a subject experiencing a cancer, wherein the cancer is expressing the YB1 protein, the method comprising administering to the subject in need thereof a pharmaceutically effective amount of a compound of Formula (I) as described in any of claims 1-38, or a pharmaceutically acceptable salt, pharmaceutically acceptable co-crystal, ester, solvate, hydrate, isomer, tautomer, isotope, polymorph, or prodrug thereof.

42. A method of sensitizing cancer cells expressing the YB1 protein in a subject to treatment with an anticancer agent, the method comprising administering to the subject in need thereof a pharmaceutically effective amount of a compound of Formula (I) as described in any of claims 1-38, or a pharmaceutically acceptable salt, pharmaceutically acceptable co-crystal, ester, solvate, hydrate, isomer, tautomer, isotope, polymorph, or prodrug thereof.

43. A method of sensitizing cancer cells expressing the YB1 protein in a subject to treatment with radiation, the method comprising administering to the subject in need thereof a pharmaceutically effective amount of a compound of Formula (I) as described in any of claims 1-38, or a pharmaceutically acceptable salt, pharmaceutically acceptable co-crystal, ester, solvate, hydrate, isomer, tautomer, isotope, polymorph, or prodrug thereof.

44. The method of any of claims 41, 42, and 43, wherein the cancer expressing YB1 protein is selected from the group of a gynecological cancer (including ovarian, endometrial, fallopian tube, and cervical cancers), breast cancers, lung cancers, prostate cancer, colorectal cancer, bladder cancer, melanoma, liver cancer, multiple myeloma, soft tissue sarcoma, osteosarcoma, Ewing's sarcoma, glioblastoma, acute myeloid leukemia, Chronic myelogenous leukemia, acute lymphoblastic leukemia, chronic lymphocytic leukemia, lymphoma, kidney cancer, renal cell carcinoma, osteosarcoma, pancreatic cancer, head and neck cancer, nasopharyngeal carcinoma, and gastric cancer.

45. The method of any of claims 1-44, wherein the compound of Formula (I) is 9-(3-fluorophenyl)-5-(2-hydroxyethyl)-6,9-dihydro-[1,3]dioxolo[4,5-g]furo[3,4-b]quinolin-8(5H)-one (SU056), or a pharmaceutically acceptable salt, pharmaceutically acceptable co-crystal, ester, solvate, hydrate, isomer, tautomer, isotope, polymorph, or prodrug thereof.


SEQUENCE LISTING (4 sequences)

SEQ ID NO: 1 - DNA, 21 bp, Artificial Sequence
  pLV[shRNA]-EFGPT2APuro-U6>hYBX1[shRNA#1]
cctgttaataaaggtcttaaa

SEQ ID NO: 2 - DNA, 21 bp, Artificial Sequence
  pLV[shRNA]-EFGPT2APuro-U6>hYBX1[shRNA#2]
ccagttcaaggcagtaaatat

SEQ ID NO: 3 - DNA, 21 bp, Artificial Sequence
  pLV[shRNA]-EGFPT2APuro-U6>Scramble[shRNA#1]
cctaaggttaagtcgccctcg

SEQ ID NO: 4 - protein, 348 aa, Artificial Sequence
  Recombinant human YB-1 protein (His Tag) (Novus Biological Cat. No. NBP2-30101)
MGSSHHHHHHSSGLVPRGSHMGSMSSEAETQQPPAAPPAAPASLSAADTKPGTTGSGAGSGGPGGLETSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE